(19) United States
(12) Patent Application Publication
(10) Pub. No.: US 2022/0356163 A1
(43) Pub. Date: Nov. 10, 2022

(54) SMALL MOLECULE INHIBITORS OF Id PROTEINS

(71) Applicant: MEMORIAL SLOAN KETTERING CANCER CENTER, New York, NY (US)

(72) Inventors: Robert BENEZRA, New York, NY (US); Ouathek OUERFELLI, New York, NY (US); Guangli YANG, New York, NY (US)

(73) Assignee: MEMORIAL SLOAN KETTERING CANCER CENTER, New York, NY (US)

(21) Appl. No.: 17/766,027

(22) PCT Filed: Sep. 30, 2020
(86) PCT No.: PCT/US2020/053468

§ 371 Date: Apr. 1, 2022

Related U.S. Application Data
(60) Provisional application No. 62/909,036, filed on Oct. 1, 2019.

(51) Int. Cl. C07D 317/58; C07C 217/60; C07C 217/62; C07C 211/49; A61P 35/00

(52) U.S. Cl. CPC C07D 317/58; C07C 217/60; C07C 217/62; C07C 211/49; A61P 35/00

(57) ABSTRACT

The present technology relates generally to compounds, compositions, and methods useful for treating, preventing, and/or ameliorating pathogenic cellular proliferation, angiogenesis, cancer, metastatic disease, and/or a pathogenic vascular proliferative disease in a subject.

### CROSS-REFERENCE TO RELATED APPLICATIONS

This application claims the benefit of and priority to U.S. Provisional Application No. 62/909,036, filed Oct. 1, 2019, the contents of which are incorporated by reference in their entirety for any and all purposes.

### U.S. GOVERNMENT SUPPORT

This invention was made with government support under CA008748 awarded by the National Institutes of Health. The government has certain rights in the invention.

### TECHNICAL FIELD

The present technology relates generally to compounds that inhibit Id (inhibitor of differentiation) proteins, compositions thereof, and methods thereof that are useful for treating, preventing, and/or ameliorating pathogenic cellular proliferation, angiogenesis, cancer, metastatic disease, and/or a pathogenic vascular proliferative disease in a subject.

### SUMMARY

In an aspect, the present disclosure provides a compound according to Formula I

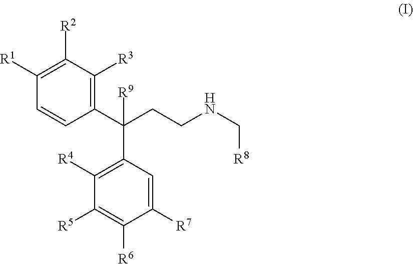

or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹⁰)(R¹¹);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹²)(R¹³);
R⁸, is aryl or heteroaryl;
R⁹ is H, C₁-C₃ alkyl, or fluoro; and
R¹⁰, R¹¹, R¹², and R¹³ are each independently C₁-C₃ alkyl.

In a related aspect, a pharmaceutical composition is provided that includes an effective amount of a compound of Formula I for treating pathogenic cellular proliferation, angiogenesis, cancer, metastatic disease, and/or a pathogenic vascular proliferative disease in a subject; and a pharmaceutically acceptable carrier.

In an aspect, a method for treating a condition in a subject is provided, where the method includes administering a compound of Formula I to the subject in an amount effective to treat the condition, where the condition includes one or more of pathogenic cellular proliferation, angiogenesis, cancer, metastatic disease, and/or a pathogenic vascular proliferative disease.

Further aspects and embodiments of the present technology are described herein.

### BRIEF DESCRIPTION OF THE DRAWINGS

FIGS. 1A-1C demonstrate that genetic deletion of Id1 or Id3 suppresses neovascularization in mouse models of wet age-related macular degeneration (AMD) Id1^{-/-} (FIG. 1A) or Id3^{-/-} (FIG. 1B) mice and littermate control Id1^{+/+} or Id3^{+/+} mice (n=12 for each group) had laser-induced rupture of Bruch's membrane at three locations in one eye. After 14 days the mean area of choroidal neovascularization (NV) was significantly less in Id1^{-/-} or Id3^{-/-} than in their corresponding controls (* indicating p<0.03 by unpaired t-test, error bars represent SEM). FIG. 1C shows the eschemia-induced retinal neovascularization in wild type, Id1^{-/-} or Id3^{-/-} mice. Id1^{-/-} or Id3^{-/-} mice and littermate control Id1^{+/+} or Id3^{+/+} mice were placed in a 75% O₂ chamber at day P7, for five days, to induce retinopathy of prematurity (ROP). At day P17 the animals were euthanized and the area of retinal neovascularization was assessed by Griffonia simplicifolia lectin staining (selective for vascular cells) of choroidal flat mounts (representative images of staining are shown) (white scale bar=500 μm). Quantification of the mean total area of NV per group is plotted with n=number of pups and * indicating p<0.0001 by unpaired t-test, error bars represent SEM).

FIGS. 2A-2G show the identification of AGX51. FIG. 2A shows the crystal structure of Id1 and E47 with hydrogen bonds depicted with black dashed lines and salt bridges depicted by gray dashed lines with the participating residues labeled. FIG. 2B shows the hydrophobic crevice analysis of Id1. Black arrow and grey region indicating the identified cleft. FIG. 2C shows the in vitro electrophoretic mobility shift assay (EMSA) of compounds A, B and C. Wedges indicate increasing concentrations of small-molecule from 1-100 μM: lanes 3-5: 20, 50 100 μM compound A; lanes 6-8: 20, 50 100 μM compound B; lanes 10-16: 1, 5, 10, 20, 30, 50, 100 μM compound C. FIG. 2D shows the structures of compounds A, B and C from FIG. 2C, where C is AGX51, red asterisk indicating the stereocenter. FIG. 2E shows the prediction of AGX51 (depicted by blue stick figure) docking site (depicted in grey) at the Id1 HLH domain (depicted in green). FIG. 2F shows the prediction of Id1 residues interacting with AGX51. FIG. 2G shows the Circular Dichroism (CD) of Id1 with DMSO (left plot), or AGX51 (0, 10, 20 and 50 μM) (middle plot), and E47 with AGX51 (0, 10, 20 and 50 μM) (right plot). See also FIGS. 8A-10C and 16-18.

FIGS. 3A-3D shows the effects of AGX51 on ID protein levels, E protein interactions and ID1 ubiquitylation. FIG. 3A shows a western blot for ID1 and ID3 on whole cell lysates from HUVECs treated with 0-40 μM AGX51 for 24 hours. FIG. 3B shows a western blot for Flag on whole cell lysates from HCT116 cells (expressing Flag-tagged ID1) treated with 60 μM AGX51 for 0-24 hours. FIG. 3C shows the ubiquitylation assay on U87MG and HCT116 cells treated with MG132 and AGX51. FIG. 3D shows the immunoprecipitation (IP) for endogenous ID1 and E47 in HCT116 cells treated with 60 μM AGX1 for one hour, with corresponding immunoblots on whole cell lysates to the right of the IP blots. See also FIGS. 11A-11C.

FIGS. 4A-4F demonstrate the effects of AGX51 on HUVEC growth. FIG. 4A shows the cell growth of HUVECs treated with DMSO or 20 μM AGX51 for 5 days. FIG. 4B shows the cell cycle analysis of HUVECs treated with DMSO or 20 μM AGX51 for 24 hours. FIG. 4C shows a western blot for Cyclin D1 on whole cell lysates from HUVECs treated with 0-40 μM AGX51 for 24 hours. Tubulin is used as a protein loading control. See also FIG. 12. FIG. 4D shows the HUVEC branching was observed after 18-20 hours of culturing on matrigel in the absence or presence of 0-40 μM AGX51, images taken at 10× magnification. FIG. 4E shows the quantification of the number of nodes, junctions, meshes and total branching length (n=4 replicates per concentration tested), * indicates p<0.05 by Wilcoxon test. FIG. 4F shows the effect of AGX51 on the migration of HUVEC. HUVEC monolayers were scratched and then media was replaced with media containing 0-40 μM AGX51 and migration was observed after 24 hours, images taken at 20× magnification.

FIGS. 5A-5F demonstrate that AGX51 treatment suppresses ocular neovascularization in mouse models of AMD and ROP. FIG. 5A shows the effect of AGX51 on laser-induced choroidal neovasculization. Wild type mice had rupture of Bruch's membrane at three locations in each eye followed by intravitreal injection of 10 μg of AGX51 or vehicle in one eye immediately and after seven days (n=10 per group). Fourteen days after laser-induced rupture, the mean area of CNV was significantly less in AGX51-injected eyes than control eyes (* indicates p<0.05 by ANOVA with Bonferroni correction for multiple comparisons, error bars show SEM). FIG. 5B shows the representative Griffonia simplicifolia lectin (marking vascular cells)-stained choroidal flat mounts from a control-injected eye and an AGX51-injected eye (bar=100 μm). FIG. 5C shows that twice daily i.p. injection of 500 μg of AGX51 also significantly suppressed CNV (n=10 mice per group, * indicates p<0.05 by unpaired t-test, error bars show SEM). FIG. 5D shows the representative Griffonia simplicifolia lectin-stained choroidal flat mounts of eyes from mice treated with AGX51 (10 μg) or vehicle by twice daily i.p. injection (bar=100 μm). FIG. 5E shows the images illustrating immunofluorescence for Id1 of CNV regions from mice treated with AGX51 or DMSO by intravitreal injection (bar=50 μm). FIG. 5F shows the effect of AGX51 on eschemia-induced retinal neovascularization. Pups (n=15) were placed in a 75% O₂ chamber at day P7 to induce ROP. At day P12, the mice were returned to room air and received intravitreal injection of 10 μg of AGX51 in one eye or DMSO in the FE. On day P17, the mice were euthanized and the area of retinal neovascularization (RNV) was assessed (* indicates p<0.01 by unpaired t-test, error bars show SEM). See also FIGS. 13 and 17.

FIGS. 6A-6D demonstrate the effects of AGX51 enantiomers alone and in combination treatment with Aflibercept. FIG. 6A shows the effect of AGX51 enantiomers on laser-induced CNV. Laser-induced CNV was induced in mice and they were treated with DMSO, AGX51 racemate, or the enantiomers (AGX51E1 or AGX51E2), n=10 mice per group. Mice were treated via i.p. injection with 50 μL of vehicle or 10 mg/mL compound bid. On day 14, the animals were euthanized and area of CNV was measured as described (* indicates p<0.05, ** indicates p=0.0014 by ANOVA and Bonferroni correction for multiple comparisons, error bars show SEM). FIG. 6B shows the representative Griffonia simplicifolia lectin (vascular cell marker)-stained choroidal flat mounts of eyes from mice treated in (a) (bar=100 μm). FIG. 6C (top panel) shows the Griffonia simplicifolia lectin-stained choroidal flat mounts of eyes of mice subjected to laser-induced CNV and treated with AGX51E2. Eight mice per group were treated by intravitreal injection with 1, 3, 10 or 30 μg AGX51E2 on days 1 and 7 following laser-induced CNV. On day 14 the mice were euthanized and the area of CNV was measured. Representative Griffonia simplicifolia lectin-stained choroidal flat mounts of eyes are shown. FIG. 6C (bottom panel) shows the quantification of data is plotted with * indicating p<0.05 by ANOVA, error bars show SEM) (bar=100 μm). FIG. 6D shows the effect of AGX51 alone and in combination with Aflibercept on laser-induced CNV. Laser-induced CNV was induced in mice and on days 1 and 7 they were treated by intravitreal injection with DMSO, AGX51 (10 μg), Aflibercept (A) (40 μg) or AGX51+Aflibercept, n=10 mice per group. On day 14 the animals were euthanized and area of CNV was measured as described. In FIGS. 6C-6D, FE refers to “Fellow Eye” and is defined as the untreated eye in an animal in which both eyes received the laser treatment. (* indicates p<0.05, ** indicates p=0.0014, and *** indicates p<0.0001 by ANOVA, error bars show SEM). See also FIG. 14.

FIGS. 7A-7E shows the characterization of AGX-A, an Id inhibiting compound of the present technology which surprisingly and unexpectedly exhibits significantly superior effects over known Id inhibitors such as AGX51. FIG. 7A shows the chemical structure of AGX-A. FIG. 7B shows the Circular Dichroism (CD) spectra demonstrating the interaction of AGX-A (0-110 μM in DMSO) and Id1. FIG. 7C shows a western blot for ID1 on whole cell lysates from HCT116 cells treated with 0-10 μM AGX-A for 24 hours. The IC₅₀ is indicated. IC₅₀ of AGX51 was 22.28 μM. FIG. 7D shows a western blot for Id1 on whole cell lysates from 4T1 cells treated with 0-20 μM AGX-A for 24 hours. The IC₅₀ is indicated. IC₅₀ of AGX51 was 26.66 μM. FIG. 7E shows the effect of AGX-A or AGX51 on laser-induced choroidal neovascularization. Laser-induced choroidal neovascularization (NV) was induced in mice and they were treated by intravitreal injection with DMSO, 1 or 5 μg AGX-A, or 1 or 5 μg AGX51. On day 14 the animals were euthanized and area of CNV was measured (** indicates p<0.01, *** indicates p<0.0001 by ANOVA, error bars show SEM).

FIGS. 8A-8C show the investigation of the physical interaction between AGX51-XL2 and Id1. FIG. 8A shows the conservation of the Id HLH domain across Id family members (Id1-Id4) in humans (hs) and mice (mm). Drosophila melanogaster (dm) Id ortholog is also shown (emc). FIG. 8B shows the structure of AGX51-XL2. FIG. 8C shows the schematic of the Id1 helix and loop regions. Black dots indicate the amino acids predicted to be in close proximity to the Id1 binding pocket, and orange dots indicate amino acids identified by mass spectrometry as being covalently bound to Id1. One sample was analyzed per group (+/−UV, +/−AGX51-XL2). The experiment was performed twice, three months apart. See also FIGS. 17-18.

FIG. 9 shows the circular dichroism of AGX51 and Id3. Circular dichroism (CD) of Id3 with 0 or 100 μM AGX51 is shown.

FIGS. 10A-10C show the NanoBRET™ assay with NanoLuc-ID1 and AGX tracer. FIG. 10A shows the structure of the AGX51 tracer. FIG. 10B shows the results of the NanoBRET™ assay. 293T cells transfected with NanoLuc-ID1 were treated with 0-4 μM AGX fluorescent tracer. Bioluminescence Resonance Energy Transfer (BRET) ratio was plotted as a function of AGX51 tracer concentration. FIG. 10C shows the results of the NanoBRET™ assay performed in the presence of AGX51 tracer and the indicated drug. 293T cells transfected with NanoLuc-ID1 were treated with 2 μM AGX fluorescent tracer and 0-60 μM AGX51 or AGX-A in the presence of 50 μg/mL digitonin. Complete substrate was added to the cells and donor and acceptor emission wavelengths measured using the GloMax Discover System luminometer and BRET ratio determined.

FIGS. 11A-11C demonstrate the effects of AGX51 on ID proteins in HCT116 cells. FIG. 11A shows a western blot for ID1, ID2, ID3 and ID4 on whole cell lysates from HCT116 cells treated with 40 μM AGX51 for 0-48 hours. Tubulin was used as a protein loading control. FIG. 11B shows the qRT-PCR analysis for ID1 and ID3 in HCT116 cells treated with 40 μM AGX51 for 24 hours. Mean fold difference with error bars showing SEM of technical replicates. FIG. 11C shows the EMSA on whole cell lysates from HCT116 cells treated with 40 μM AGX51 for 1 or 24 hours. The space indicates that the samples were run on different gels or different parts of a gel.

FIGS. 12A-12C demonstrate the effects of AGX51 on HCT116 cells in culture. FIG. 12A shows the MTT cell viability assay on HCT116 cells treated with 40 μM AGX51 for 24 hours. Mean value of technical triplicates is plotted with error bars representing SD. FIG. 12B shows the cell cycle analysis on HCT116 cells treated with 40 μM AGX51 for 4-24 hours. FIG. 12C shows a western blot for Cyclin D1 on lysates from HCT116 cells treated with 40 μM AGX51, or vehicle for 0-48 hours. Tubulin was used as a protein loading control. The space indicates that the samples were run on different gels or different parts of a gel.

FIG. 13 shows the dose titration of AGX51. Laser-induced CNV was induced in mice and on days 1 and 7 they were treated by intravitreal injection with DMSO (Control) (n=7), AGX51 (1 μg) (n=8) or AGX51 (5 μg) (n=7). On day 14, the animals were euthanized and area of CNV was measured as described (* indicates p<0.05 by ANOVA, error bars show SEM).

FIGS. 14A-14C show the Circular dichroism of AGX51 enantiomers. Circular dichroism (CD) of Id1 with 0 or 100 μM AGX51 (FIG. 14A), AGX51-E2 (FIG. 14B) and AGX51-E1 (FIG. 14C) are shown.

FIG. 15 shows the sources of reagents and resources used in studies described in the working examples.

FIG. 16 shows a summary of crystallographic diffraction data collection and refinement statistics, according to the working examples.

FIG. 17 shows the Id1 amino acids identified as covalently bound to AGX51-XL2 by mass spectrometry, according to the working examples.

FIG. 18 shows the Id1 amino acids identified as covalently bound to AGX51-XL2 by mass spectrometry with and without AGX51 competition, according to the working examples.

FIG. 19 shows the results of toxicity testing following two week treatment with 60 mg/kg AGX51 administered twice daily by intraperitoneal injection, according to the working examples.

FIG. 20 shows the X-Ray Structure of R-(+)-3-(benzo[d][1,3]dioxol-5-yl)-N-benzyl-3-(2-methoxyphenyl)propan-1-amine, according to the working examples.

FIG. 21 illustrates side-by-side comparative effects of AGX51 and AGX-A for knockdown of Id1 in TFK1 cell cultures, according to the working examples.

FIG. 22 illustrates side-by-side comparative effects of AGX51 and AGX-A for knockdown of Id3 in TFK1 cell cultures, according to the working examples.

FIG. 23 illustrates the dose-dependent effects of AGX51 and AGX-A on TFK1 cell viability over a 24 hour period, according to the working examples.

FIG. 24 provides results of a murine model of cholangiocarcinoma showing the in vivo efficacy of AGX-A in DMSO and a combination therapy utilizing gemcitabine and AGX-A in DMSO, according to the working examples.

FIGS. 25A-25B which provide the results of 24 hour cell viability studies for SNU1079 cells (FIG. 25A) and SNU1196 cells (FIG. 25B) treated with AGX-A in an aqueous solution including 2-hydroxypropyl-β-cyclodextrin (“HPBCD”), according to the working examples.

FIG. 26 provides results of a murine model of cholangiocarcinoma showing the in vivo efficacy of AGX-A in aqueous solution (including 12.5% by weight HPBCD) and a combination therapy utilizing gemcitabine and the aqueous solution of AGX-A, according to the working examples.

FIG. 27 provides the results of Alamar blue viability assays for 4T1 cells treated with AGX51 to aid in future comparisons with compounds of the present technology, according to the working examples. It is expected that compounds of the present technology will exhibit similar or significantly improved effects.

FIG. 28 illustrates the effects of AGX51 against mouse pancreatic organoid cell lines T7 and T8 to aid in future comparisons with compounds of the present technology, according to the working examples. It is expected that compounds of the present technology will exhibit similar or significantly improved effects.

FIG. 29 illustrates the effects of AGX51 against mouse pancreatic cancer lines 806 (KrasG12D; Ink4a−/−; Smad4−/−), NB44 (KrasG12D; Ink4a−/−) and 4279 (KrasG12D; Ink4a−/−) to aid in future comparisons with compounds of the present technology, according to the working examples. It is expected that compounds of the present technology will exhibit similar or significantly improved effects.

FIG. 30 illustrates the effects of AGX51 against human pancreatic cancer cell line Panc1 and A21 to aid in future comparisons with compounds of the present technology, according to the working examples. It is expected that compounds of the present technology will exhibit similar or significantly improved effects.

FIG. 31 illustrates the effects of AGX51 (with and without paclitaxel) on primary tumors to aid in future comparisons with compounds of the present technology, according to the working examples. It is expected that compounds of the present technology will exhibit similar or significantly improved effects.

FIG. 32 illustrates the effects of AGX51 on lung colonization to aid in future comparisons with compounds of the present technology, according to the working examples. It is expected that compounds of the present technology will exhibit similar or significantly improved effects.

FIG. 33 illustrates the effects of AGX51 on established lung metastases to aid in future comparisons with compounds of the present technology, according to the working examples. It is expected that compounds of the present technology will exhibit similar or significantly improved effects.

FIGS. 34A-34B illustrate the results of studies assessing the inhibition of the extravasation and initial seeding at the secondary site or the progression of extravasated cancer cell outgrowth into tumors via treatment with AGX51 to aid in future comparisons with compounds of the present technology, according to the working examples. The effects of AGX51 on initial seeding of the cancer cells at the secondary site is shown in FIG. 34A in terms of total cells and in FIG. 34B in terms of total tissue area. It is expected that compounds of the present technology will exhibit similar or significantly improved effects.

FIGS. 35A-35D illustrate the effects of AGX51 on sporadic tumor to aid in future comparisons with compounds of the present technology, according to the working examples. FIG. 35A provides the results in terms of total number of tumors; FIG. 35B illustrates the resulting number of tumors measuring <1 mm; FIG. 35C illustrates the resulting number of tumors measuring from 1.5 mm to 2.5 mm; and FIG. 35D illustrates the number of tumors measuring ≥3 mm. It is expected that compounds of the present technology will exhibit similar or significantly improved effects.

### DETAILED DESCRIPTION

It is to be appreciated that certain aspects, modes, embodiments, variations and features of the present methods are described below in various levels of detail in order to provide a substantial understanding of the present technology.

### Definitions

Unless defined otherwise, all technical and scientific terms used herein generally have the same meaning as commonly understood by one of ordinary skill in the art to which this technology belongs.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The phrase “and/or” as used in the present disclosure will be understood to mean any one of the recited members individually or a combination of any two or more thereof—for example, “A, B, and/or C” would mean “A, B, C, A and B, A and C, or B and C.”

As used herein, the terms “cancer,” “neoplasm,” and “tumor,” are used interchangeably and refer to cells that have undergone a malignant transformation that makes them pathological to the host organism. Primary cancer cells (that is, cells obtained from near the site of malignant transformation) may be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “metastasis” or “metastatic” refers to the ability of a cancer cell to invade surrounding tissues, to enter the circulatory system and to establish malignant growths at new sites.

“Non-Metastatic” refers to tumors that do not spread beyond their original site of development and specifically do not enter the circulatory system and establish malignant growths at new sites.

As used herein, “prevention,” “prevent,” or “preventing” of a disease or condition refers to one or more compounds that, in a statistical sample, reduces the occurrence of the disease or condition in the treated sample relative to an untreated control sample, or delays the onset of one or more symptoms of the disease or condition relative to the untreated control sample. As used herein, prevention includes preventing or delaying the initiation of symptoms of the disease or condition. As used herein, prevention also includes preventing a recurrence of one or more signs or symptoms of a disease or condition.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, N.Y., (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁹² groups, and “sulfonyls” include —SO₂OR⁸³. R⁸⁰, R⁸¹, R⁸², and R¹³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R¹⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

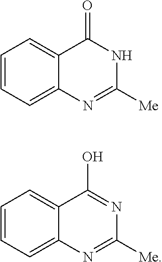 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

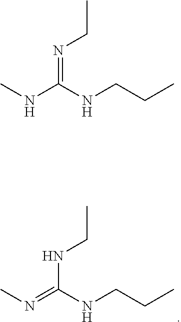

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents, and published patent specifications are referenced by an identifying citation. Also within this disclosure are Arabic numerals referring to referenced citations, the full bibliographic details of which are provided immediately preceding the claims. The disclosures of these publications, patents, and published patent specifications are hereby incorporated by reference into the present disclosure to more fully describe the state of the art to which this invention pertains.

### The Present Technology

As described in the art (for example, in U.S. Pat. Publ. No. 2009/0226422 and Int'l Publ. No. WO 2015/089495, each of which is incorporated herein by reference), Id proteins have been shown to play key roles as regulators of stem cell identity in both colorectal cancer and malignant glioma, essential for both self-renewal and tumor-initiating capacity of cancer stem cells. Despite the mechanistic complexity and indefinite pathways involved in cancer stem cell development, anti-Id compounds disable Id proteins at a critical foundation to disrupt stem cell identity and impair stem cell tumor initiation. Anti-metastatic and anti-angiogenic effective anti-Id compounds may specifically target tumor stem cell viability, proliferation capacity, tumor-initiation potential, and/or cell fate determination—with the result of substantially decreasing populations of new tumor induction-competent stem cells present in new or established tumors.

The compounds of the present technology target Id1 and Id3 positive “resting” stem cells. These cells representing a pool of cancer progenitor cells relatively resistant to chemotherapy (based on their resting, non-proliferative state, such cells elude first line chemotherapy targeting proliferative cells). As such these stem cells frequently escape first line cancer treatment, whereafter they are capable of rebounding to give rise to new cancer cell populations. Yet additional evidence presented here shows that compounds of the present technology also penetrate in their effects to preclude acquired resistance, either alone or in combination with conventional chemotherapeutic cancer therapies. For example, resting stem cells or cancer cells that mutationally escape first line treatment (e.g., cells that acquire resistance to chemotherapeutic drugs through mutation) cannot further elude or develop resistance to the compounds of the present technology. Without being bound by theory, the functionally critical, evolutionarily constrained/conserved Id binding interface (targeted by the compounds of the present technology) is practically immutable to generate “acquired resistance” because any structural mutation yields a biologically inoperative Id protein, functionally null for the essential purposes such proteins serve in cancer cells.

Thus, in one aspect, the present disclosure provides a compound according to Formula I

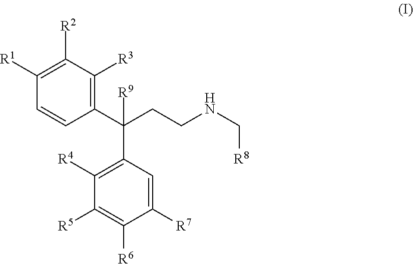

or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹⁰)(R¹¹);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹²)(R¹³);
R⁸, is aryl or heteroaryl;
R⁹ is H, C₁-C₃ alkyl, or fluoro; and
R¹⁰, R¹¹, R¹², and R¹³ are each independently C₁-C₃ alkyl.

In any embodiment herein, it may be that R¹, R², and R³ are each independently H, C₁-C₃ alkyl, C₁-C₃ alkoxy, trifluoromethyl, trifluoromethoxy, halo, or —N(R¹⁰)(R¹¹). In any embodiment herein, it may be that R¹, R², and R³ are each independently H, C₁-C₃ alkyl, C₁-C₃ alkoxy, halo, or —N(Me)₂. In any embodiment herein, it may be that R¹, R², and R³ are each independently H, methyl, methoxy, isopropyl, isopropoxy, fluoro, or —N(Me)₂. In any embodiment herein, it may be that R³ is methoxy.

In any embodiment herein, it may be that R⁴, R⁵, R⁶, and R⁷ are each independently H, C₁-C₃ alkyl, C₁-C₃ alkoxy, trifluoromethyl, trifluoromethoxy, halo, or —N(R¹²)(R¹³). In any embodiment herein, it may be that R⁴, R⁵, R⁶, and R⁷ are each independently H, C₁-C₃ alkyl, C₁-C₃ alkoxy, halo, or —N(Me)₂. In any embodiment herein, it may be that R⁴, R⁵, R⁰, and R⁷ are each independently H, methyl, methoxy, isopropyl, isopropoxy, fluoro, or —N(Me)₂. In any embodiment herein, it may be that R⁶ is isopropoxy.

In any embodiment herein, it may be that the compound of Formula I is a compound of Formula IA

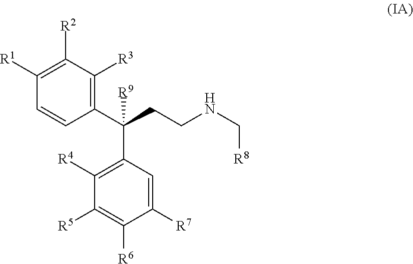

or a pharmaceutically acceptable salt and/or solvate thereof.

In any embodiment herein, it may be that the compound of Formula I is a compound of Formula IB

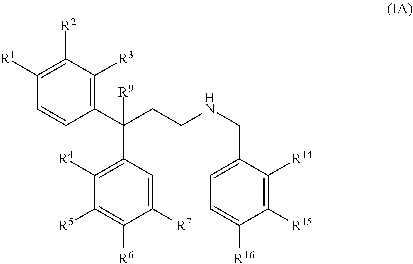

or a pharmaceutically acceptable salt and/or solvate thereof, wherein R¹⁴, R¹⁵, and R¹⁶ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.

In any embodiment herein, it may be that R¹⁴, R¹⁵, and R¹⁶ are each independently H, C₁-C₃ alkyl, C₁-C₃ alkoxy, trifluoromethyl, trifluoromethoxy, halo, aryloxy, aryloyl, or —N(C₁-C₃ alkyl)₂. In any embodiment herein, it may be that R¹⁴, R¹⁵, and R¹⁶ are each independently H, C₁-C₃ alkyl, C₁-C₃ alkoxy, trifluoromethyl, trifluoromethoxy, halo, or —N(Me)₂. In any embodiment herein, it may be that R¹⁴, R¹⁵, and R¹⁶ are each independently H, methyl, methoxy, isopropyl, isopropoxy, fluoro, or —N(Me)₂. In any embodiment herein, it may be that R¹⁴, R¹⁵, and R¹⁶ are each independently H.

In any embodiment herein, it may be that the compound of Formula I is

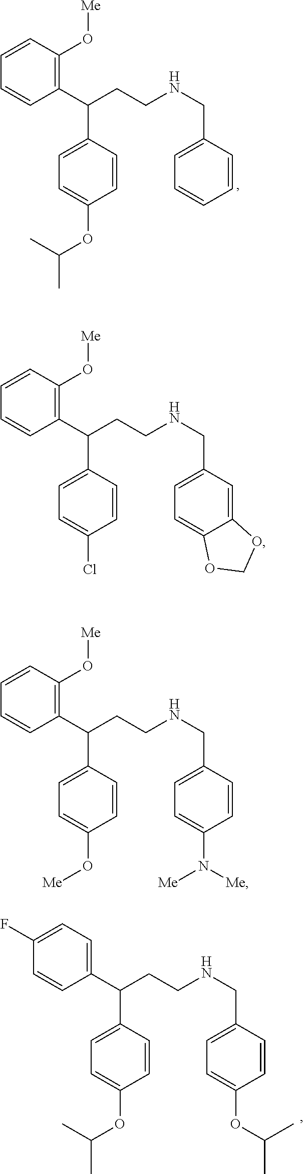

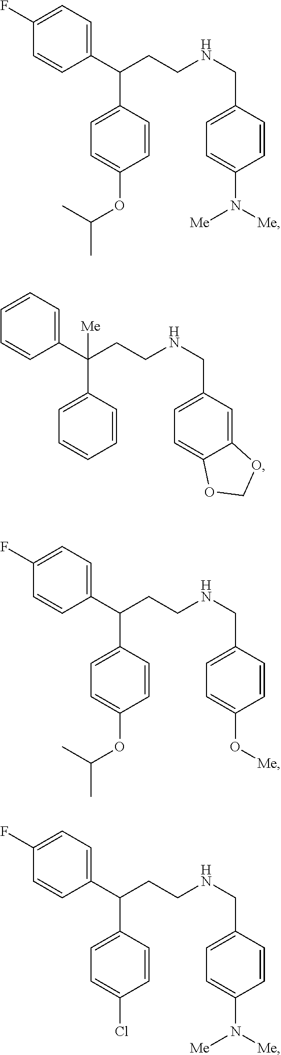

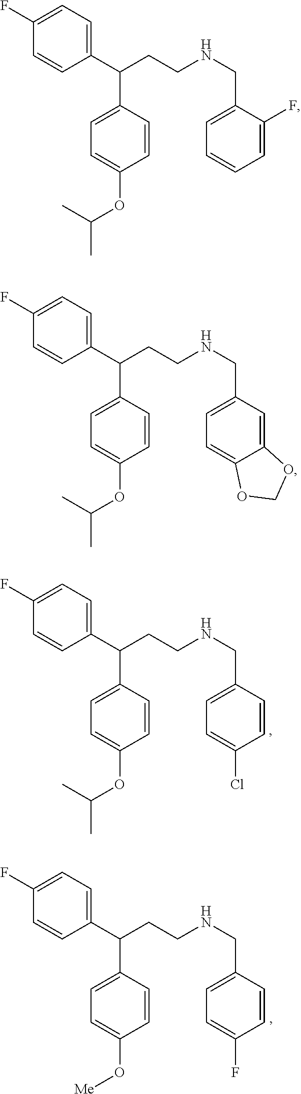 [SMILES: CC(C)Oc1ccc(C(CCNCc2ccccc2F)c2ccc(F)cc2)cc1 ; CC(C)Oc1ccc(C(CCNCc2ccc3c(c2)OCO3)c2ccc(F)cc2)cc1 ; CC(C)Oc1ccc(C(CCNCc2ccc(Cl)cc2)c2ccc(F)cc2)cc1 ; COc1ccc(C(CCNCc2ccc(F)cc2)c2ccc(F)cc2)cc1]

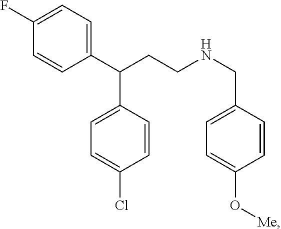 [SMILES: COc1ccc(CNCCC(c2ccc(F)cc2)c2ccc(Cl)cc2)cc1]

or a pharmaceutically acceptable salt and/or solvate thereof.

In any embodiment herein, it may be that the compound of Formula I is a compound of Formula IC

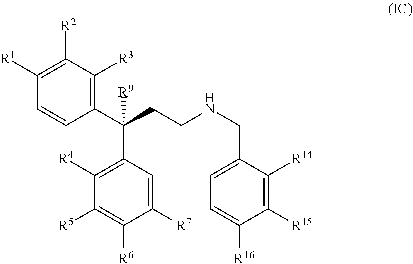

or a pharmaceutically acceptable salt and/or solvate thereof.

In any embodiment herein, it may be that the compound of Formula I is

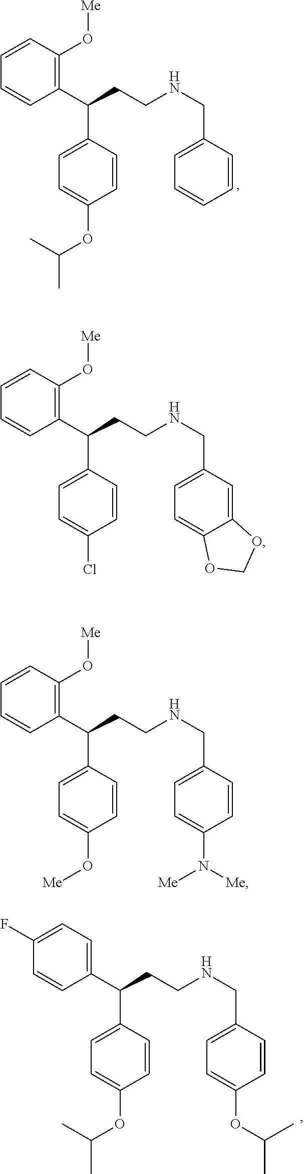

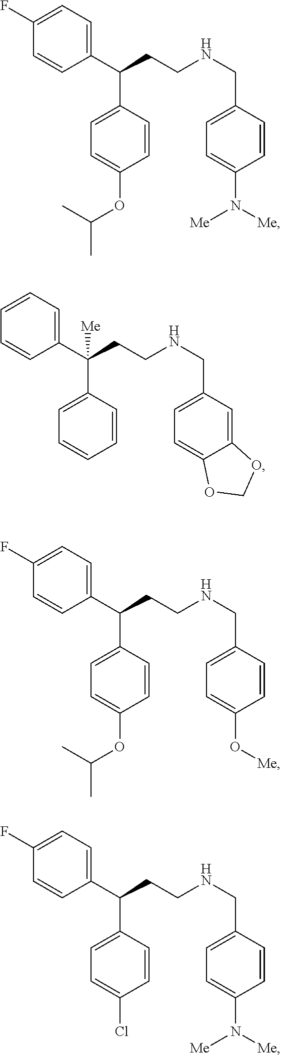

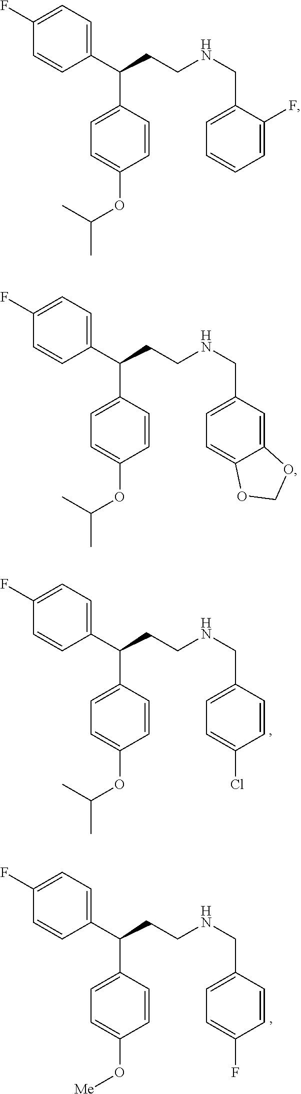 [SMILES: CC(C)Oc1ccc([C@H](CCNCc2ccccc2F)c2ccc(F)cc2)cc1 ; CC(C)Oc1ccc([C@H](CCNCc2ccc3c(c2)OCO3)c2ccc(F)cc2)cc1 ; CC(C)Oc1ccc([C@H](CCNCc2ccc(Cl)cc2)c2ccc(F)cc2)cc1 ; COc1ccc([C@H](CCNCc2ccc(F)cc2)c2ccc(F)cc2)cc1]

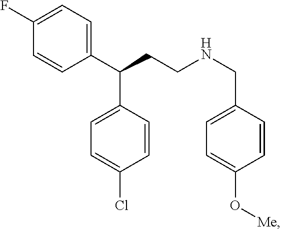 [SMILES: COc1ccc(CNCC[C@H](c2ccc(F)cc2)c2ccc(Cl)cc2)cc1]

or a pharmaceutically acceptable salt and/or solvate thereof.

In an aspect, a composition is provided that includes a compound of Formula I of any embodiment disclosed herein (e.g., a compound according to Formula I, a compound disclosed above, a pharmaceutically acceptable salt and/or solvate of any compound disclosed above) and a pharmaceutically acceptable carrier or one or more excipients, fillers or agents (collectively referred to hereafter as “pharmaceutically acceptable carrier” unless otherwise indicated and/or specified). In a related aspect, a medicament is provided that includes a compound of Formula I of any embodiment disclosed herein. In a related aspect, a pharmaceutical composition is provided that includes (i) an effective amount of a compound of Formula I of any embodiment disclosed herein, and (ii) a pharmaceutically acceptable carrier. For ease of reference, the compositions, medicaments, and pharmaceutical compositions of the present technology may collectively be referred to herein as “compositions.” In further related aspects, the present technology provides methods including a compound of any embodiment disclosed herein and/or a composition of any embodiment disclosed herein as well as uses of a compound of any embodiment disclosed herein and/or a composition of any embodiment disclosed herein. Such methods and uses may include an effective amount of a compound of any embodiment disclosed herein. In any aspect or embodiment disclosed herein (collectively referred to herein as “any embodiment herein,” “any embodiment disclosed herein,” or the like), the effective amount may be an amount that treats pathogenic cellular proliferation, angiogenesis, cancer, metastatic disease, and/or a pathogenic vascular proliferative disease in a subject. As used herein, a “subject” or “patient” is typically a mammal, such as a cat, dog, rodent or primate. Typically the subject is a human, and, preferably, a human suffering from or suspected of suffering from pathogenic cellular proliferation, angiogenesis, cancer, metastatic disease, and/or a pathogenic vascular proliferative disease. The term “subject” and “patient” can be used interchangeably.

Thus, the instant present technology provides pharmaceutical compositions and medicaments comprising any of the compounds disclosed herein of the present technology (e.g., any embodiment disclosed herein of a compound of Formula I) and a pharmaceutically acceptable carrier. The compositions may be used in the methods and treatments described herein. Such compositions and medicaments include a therapeutically effective amount of any compound as described herein, including but not limited to a compound of Formula I. The pharmaceutical composition may be packaged in unit dosage form. The unit dosage form is effective in treating pathogenic cellular proliferation, angiogenesis, cancer, metastatic disease, and/or a pathogenic vascular proliferative disease when administered to a subject in need thereof.

The pharmaceutical compositions and medicaments of the present technology may be prepared by mixing one or more compounds of the present technology, pharmaceutically acceptable salts thereof, stereoisomers thereof, tautomers thereof, or solvates thereof, with pharmaceutically acceptable carriers, excipients, binders, diluents or the like. The compounds and compositions described herein may be used to prepare formulations and medicaments that prevent or treat pathogenic cellular proliferation, angiogenesis, cancer, metastatic disease, and/or a pathogenic vascular proliferative disease. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until a desired result is achieved. The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition, and the pharmacological activity of the particular compound being used. The determination of optimum dosages for a particular patient is well known to those skilled in the art.

Various assays and model systems can be readily employed to determine the therapeutic effectiveness of the treatment according to the present technology. Effectiveness of the compositions and methods of the present technology may also be demonstrated by a decrease in symptoms.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

For example, the effectiveness of compounds, compositions, and method of the present technology against cancer and metastatic disease may be monitored in terms of clinical success by any of a variety of methods, for example by tumor imaging with x-rays or MRIs (e.g., to determine if tumors have decreased in size or number in treated patients). Effectiveness may often be determined by radiographic or MRI observation of a decrease in tumor size. Effective compounds, compositions, and method of the present technology for treating cancer will routinely yield at least a 10%, 25%, 50%, 75%, 90% or greater reduction of tumor size in treated patients, or average tumor size among a group of treated patients, compared to qualified, comparable control subjects. Effectiveness of compounds, compositions, and method of the present technology directed against cancer and metastatic disease may further be determined by measuring the number of circulating tumor cells in blood samples between suitable test and control subjects. This may be accomplished by any means applicable including, but not limited to immunomagnetic selection, flow cytometry, immunobead capture, fluorescence microscopy, cytomorphologic analysis, or cell separation technology. Effective compounds, compositions, and method of the present technology for treating cancer will routinely yield at least a 10%, 25%, 50%, 75%, 90% or greater reduction of circulating tumor cells in blood samples of treated patients, or among a group of treated patients, compared to qualified, comparable control subjects. Effectiveness of compounds, compositions, and method of the present technology directed against cancer and metastatic disease may further may also be determined by detecting or measuring primary tumor cell occurrence or number in secondary tissues or organs, including but not limited to bone, lymph nodes and lung. Effective compounds, compositions, and method of the present technology for treating cancer will routinely yield at least a 10%, 25%, 50%, 75%, 90% or greater reduction in the occurrence or number of primary tumor cells metastasized to secondary tissues or organs among treated patients compared to qualified, comparable control subjects.

In any embodiment or aspect herein, anti-angiogenic compounds, compositions, and method of the present technology may be effective to reduce pathologic ocular neovascularization in a mammalian subject. These methods may employ monotherapy or combination therapy. The compounds, compositions, and method of the present technology are “anti-angiogenic effective”, for example, to reduce incidence, size, or number of vascular lesions in an ocular tissue of a subject presenting with age-related macular degeneration (AMD). “Reducing neovascularization” may correspond to an observed reduction in a histopathologic or ocular angiography index of AMD lesion size, for example a reduced occurrence, size, number or distribution of lesions or “foci” of lesions observed at a secondary ocular site. Anti-angiogenic efficacy may be determined by a positive change in one or more patient therapeutic indices correlating with effective prevention and/or treatment of AMDs, e.g., by an increase in a time period of disease free or disease stable conditions for subjects receiving the a compound/composition of the present technology compared to suitable control subjects not receiving the compound/composition of the present technology.

Anti-AMD lesion efficacy, e.g., efficacy diminishing or stabilizing growth of the neovascular lesion complex, of the compounds, compositions, and methods of the of the present technology may yield substantial therapeutic benefits and improved treatment outcomes in patients treated for an ocular condition (or any other pathogenic condition) with harmful angiogenesis as part of its underlying pathology. For example, patients treated with the compounds, compositions, and methods of the present technology may exhibit improved treatment outcomes with no increase or an observed decrease in adverse side effects. Illustrative of these benefits, compounds, compositions, and methods of the present technology may yield at least a 20% increase in one or more positive clinical therapeutic indices for example a beneficial change in AMD lesion index (eg, a reduction in occurrence, size, number or distribution of the lesion or “foci” of the primary lesion observed at a secondary ocular site. Anti-AMD lesion efficacy of the compounds, compositions, and method of the present technology may be demonstrable indirectly by at least a 20% increase in a disease-free or disease stable condition for patients treated with compounds/compositions of the present technology compared to survival determined in suitable control patients (not treated with a compound/composition of the present technology). Compounds, compositions, and methods of the present technology may result in even greater anti-AMD clinical benefit, for example yielding a 20-50% increase in a positive therapeutic index, 50-90% increase, up to a 75%-100% increase, including total remission of observed primary AMD lesion enduring for 6 months to a year, 1-2 years, 2-5 years, 5 years or greater, including 10 year and longer remission. Compounds, compositions, and method of the present technology may be anti-AMD effective to yield at least a 20% decrease in lesion size, a 20%-50%, a 50%-75%, up to a 90% or greater decrease in lesion size, e.g., as demonstrated by comparative histopathology, ocular angiography, optical coherence tomography (OCT) or another ocular imaging technique in subjects treated with a compound/composition of the present technology versus non-treated or placebo-treated subjects. Anti-AMD efficacy may correlate with no increase or even a decrease in observed symptoms of AMD, e.g., loss of visual acuity between patients treated with compounds/compositions of the present technology and positive control-treated subjects. For example, subjects including subjects treated with monotherapy via the compounds/compositions of the present technology, and subjects treated with combinatorial methods such as compounds/compositions of the present technology plus anti-VEGF therapy may exhibit no increase in Snellen chart score and may exhibit at least a 20% increase, a 20-50% increase, up to a 50-90% or greater increase in Snellen chart score compared to positive control subjects treated with conventional (e.g., anti-VEGF) therapy.

The compounds of the present technology may also be administered to a patient along with other conventional therapeutic agents that may be useful in the treatment of pathogenic cellular proliferation, angiogenesis, cancer, metastatic disease, and/or a pathogenic vascular proliferative disease. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially or synergistically be effective for the treatment of pathogenic cellular proliferation, angiogenesis, cancer, metastatic disease, and/or a pathogenic vascular proliferative disease.

In an aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, preferably, 1×10⁻³ g/kg to 1.0 g/kg. Dosage of a compound of the present technology can also vary from 0.01 mg/kg to 100 mg/kg or, preferably, from 0.1 mg/kg to 10 mg/kg.

A compound of the present technology can also be modified, for example, by the covalent attachment of an organic moiety or conjugate to improve pharmacokinetic properties, toxicity or bioavailability (e.g., increased in vivo half-life). The conjugate can be a linear or branched hydrophilic polymeric group, fatty acid group or fatty acid ester group. A polymeric group can comprise a molecular weight that can be adjusted by one of ordinary skill in the art to improve, for example, pharmacokinetic properties, toxicity or bioavailability. Exemplary conjugates can include a polyalkane glycol (e.g., polyethylene glycol (PEG), polypropylene glycol (PPG)), carbohydrate polymer, amino acid polymer or polyvinyl pyrolidone and a fatty acid or fatty acid ester group, each of which can independently comprise from about eight to about seventy carbon atoms. Conjugates for use with a compound of the present technology can also serve as linkers to, for example, any suitable substituents or groups, radiolabels (marker or tags), halogens, proteins, enzymes, polypeptides, other therapeutic agents (for example, a pharmaceutical or drug), nucleosides, dyes, oligonucleotides, lipids, phospholipids and/or liposomes. In one aspect, conjugates can include polyethylene amine (PEI), polyglycine, hybrids of PEI and polyglycine, polyethylene glycol (PEG) or methoxypolyethylene glycol (mPEG). A conjugate can also link a compound of the present technology to, for example, a label (fluorescent or luminescent) or marker (radionuclide, radioisotope and/or isotope) to comprise a probe of the present technology. Conjugates for use with a compound of the present technology can, in one aspect, improve in vivo half-life. Other exemplary conjugates for use with a compound of the present technology as well as applications thereof and related techniques include those generally described by U.S. Pat. No. 5,672,662, which is hereby incorporated by reference herein.

In another aspect, the present technology provides methods of identifying a target of interest including contacting the target of interest with a detectable or imaging effective quantity of a labeled compound of the present technology. A detectable or imaging effective quantity is a quantity of a labeled compound of the present technology necessary to be detected by the detection method chosen. For example, a detectable quantity can be an administered amount sufficient to enable detection of binding of the labeled compound to a target of interest including, but not limited to, a cell or tissue associated with pathogenic cellular proliferation, angiogenesis, cancer, metastatic disease, and/or a pathogenic vascular proliferative disease. Suitable labels are known by those skilled in the art and can include, for example, radioisotopes, radionuclides, isotopes, fluorescent groups, biotin (in conjunction with streptavidin complexation), and chemiluminescent groups. Upon binding of the labeled compound to the target of interest, the target may be isolated, purified and further characterized such as by determining the amino acid sequence.

The terms “associated” and/or “binding” can mean a chemical or physical interaction, for example, between a compound of the present technology and a target of interest. Examples of associations or interactions include covalent bonds, ionic bonds, hydrophilic-hydrophilic interactions, hydrophobic-hydrophobic interactions and complexes. Associated can also refer generally to “binding” or “affinity” as each can be used to describe various chemical or physical interactions. Measuring binding or affinity is also routine to those skilled in the art. For example, compounds of the present technology can bind to or interact with a target of interest or precursors, portions, fragments and peptides thereof and/or their deposits.

### Combination Therapy

As indicated previously in this disclosure, in any embodiment or aspect herein, a compound, composition, or pharmaceutical composition of any embodiment of the present technology may be combined with one or more additional therapies for the prevention or treatment of pathogenic cellular proliferation, angiogenesis, cancer, metastatic disease, and/or a pathogenic vascular proliferative disease. Additional therapeutic agents include, but are not limited to, chemotherapeutic agents, immunotherapeutic agents, surgery, radiation therapy, anti-angiogenic agents, non-steroidal anti-inflammatory drugs, or any combination thereof.

Additionally or alternatively, in any of the embodiments disclosed herein, the additional therapeutic agent may be selected from the group consisting of alkylating agents, topoisomerase inhibitors, endoplasmic reticulum stress inducing agents, antimetabolites, immunotherapeutic agents, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, endocrine/hormonal agents, bisphosphonate therapy agents, phenphormin, anti-angiogenic agents, Histone deacetylase inhibitors, and non-steroidal anti-inflammatory drugs (NSAIDs).

Additionally or alternatively, in any of the embodiments disclosed herein, the additional therapeutic agent may be a chemotherapeutic agent selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, ABRAXANE® (albumin-bound paclitaxel), protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and mixtures thereof.

Additionally or alternatively, in some embodiments, the additional therapeutic agent may be an antimetabolite selected from the group consisting of 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Additionally or alternatively, in some embodiments, the additional therapeutic agent may be a taxane selected from the group consisting of accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Additionally or alternatively, in some embodiments, the additional therapeutic agent may be a DNA alkylating agent selected from the group consisting of cyclophosphamide, chlorambucil, melphalan, bendamustine, uramustine, estramustine, carmustine, lomustine, nimustine, ranimustine, streptozotocin; busulfan, mannosulfan, and mixtures thereof.

Additionally or alternatively, in some embodiments, the additional therapeutic agent may be a topoisomerase I inhibitor selected from the group consisting of SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof.

Additionally or alternatively, in some embodiments, the additional therapeutic agent may be a topoisomerase II inhibitor selected from the group consisting of amsacrine, etoposide, etoposide phosphate, teniposide, daunorubicin, mitoxantrone, amsacrine, ellipticines, aurintricarboxylic acid, doxorubicin, and HU-331 and combinations thereof.

Additionally or alternatively, in some embodiments, the additional therapeutic agent may be an immunotherapeutic agent selected from the group consisting of immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Additionally or alternatively, in some embodiments, the additional therapeutic agent may be an anti-angiogenic agent selected from the group consisting of bevacizumab, cediranib, axitinib, anginex, sunitinib, sorafenib, pazopanib, vatalanib, cabozantinib, ponatinib, lenvatinib, SU6668, Everolimus (Afinitor®), Lenalidomide (Revlimid®), Ramucirumab (Cyramza®), Regorafenib (Stivarga®), Thalidomide (Synovir, Thalomid®), Vandetanib (Caprelsa®), and Ziv-aflibercept (Zaltrap®).

Additionally or alternatively, in some embodiments, the additional therapeutic agent may be a histone deacetylase inhibitor selected from the group consisting of trichostatin A (TSA), tubacin, apicidin, depsipeptide, MS275, BML-210, RGFP966, MGCD0103, LBH589, splitomicin, FK228, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC₂₀₂, Mocetinostat, Romidepsin, Nicotinamide, Sirtinol, Cambinol, and EX-527.

Additionally or alternatively, in some embodiments, the additional therapeutic agent may be a NSAID selected from the group consisting of indomethacin, fenoprofen, ibuprofen, flufenamic acid, aspirin, celecoxib, diclofenac, diflunisal, etodolac, ketoprofen, ketorolac, nabumetone, naproxen, oxaprozin, piroxicam, salsalate, sulindac, and tolmetin.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of immunotherapeutic agents include immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

Examples of anti-angiogenic agents include bevacizumab, cediranib, axitinib, anginex, sunitinib, sorafenib, pazopanib, vatalanib, cabozantinib, ponatinib, lenvatinib, SU6668, Everolimus (Afinitor®), Lenalidomide (Revlimid®), Ramucirumab (Cyramza®), Regorafenib (Stivarga®), Thalidomide (Synovir, Thalomid®), Vandetanib (Caprelsa®), and Ziv-aflibercept (Zaltrap®).

Examples of histone deacetylase inhibitors include trichostatin A (TSA), tubacin, apicidin, depsipeptide, MS275, BML-210, RGFP966, MGCD0103, LBH589, splitomicin, FK228, phenylbutyrate, SAHA, Belinostat, Panabiostat, Givinostat, Resminostat, Abexinostat, Quisinostat, Rocilinostat, Practinostat, CHR-3996, Valproic acid, Butyric acid, Entinostat, Tacedinaline, 4SC₂₀₂, Mocetinostat, Romidepsin, Nicotinamide, Sirtinol, Cambinol, and EX-527.

Examples of NSAIDs include indomethacin, fenoprofen, ibuprofen, flufenamic acid, aspirin, celecoxib, diclofenac, diflunisal, etodolac, ketoprofen, ketorolac, nabumetone, naproxen, oxaprozin, piroxicam, salsalate, sulindac, and tolmetin.

In any case, in any embodiment herein, the multiple therapeutic agents may be administered in any order or even simultaneously. If simultaneously, the multiple therapeutic agents may be provided in a single, unified form, or in multiple forms (by way of example only, either as a single injection, as two separate injections, or as an injection in combination with pill). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits comprising compounds and/or compositions of the present technology and instructions for using the same to prevent and/or treat pathogenic cellular proliferation, angiogenesis, cancer, metastatic disease, and/or a pathogenic vascular proliferative disease. Optionally, the above described components of the kits of the present technology are packed in suitable containers and labeled for the prevention and/or treatment of pathogenic cellular proliferation, angiogenesis, cancer, metastatic disease, and/or a pathogenic vascular proliferative disease.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable carrier of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology. In any embodiment herein, the written product may instruct performance of a method according to any embodiment described herein.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology. The following Examples demonstrate the preparation, characterization, and use of illustrative compositions of the present technology that inhibit Id proteins.

### Example 1: Synthesis of AGX51

The reaction of 2-methoxycinnamaldehyde 1 and the potassium aryltrifluoroborate 2a or regular arylboronic acid 2b, provided the desired adduct 3 in high yield (Scheme 1). Reductive amination of aldehyde 3 with benzylamine and sodium borohydride afforded amine 4. Acylation of amine 4 with propionyl chloride provided the final product 5 (AGX51) in 75% overall yields.

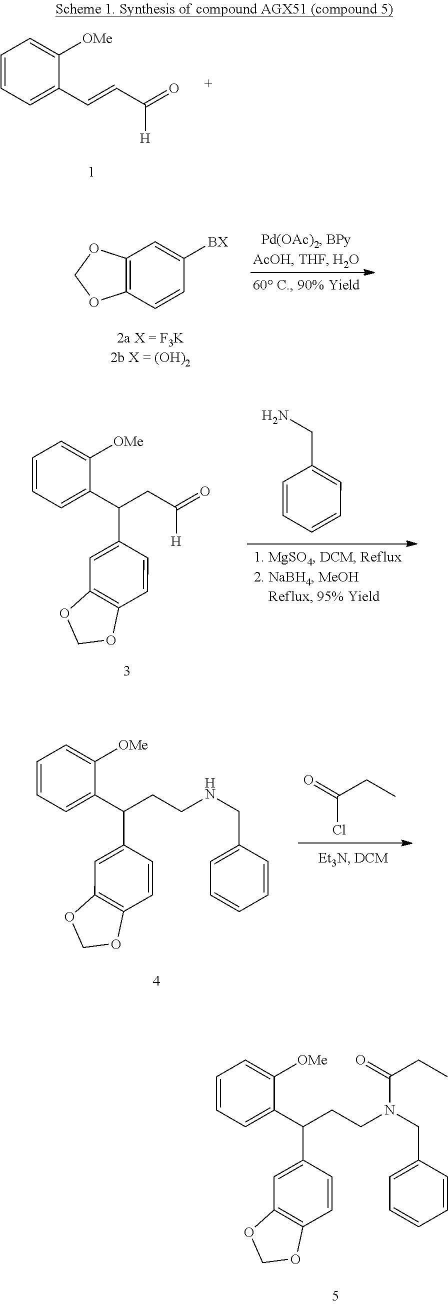

### Example 2: Synthesis of AGX-A

Compound 7 was generated in one step by conjugate addition of trifluoroborate 6 to aldehyde 1 in the presence of catalytic amount of Palladium dibenzylideneacetone and triphenylphosphine. Reductive amination of aldehyde 7 gives secondary amine 8 (AGX-A) in high yield as a viscous oil which was transformed into the corresponding hydrochloride salt 9 and was isolated as a white solid.

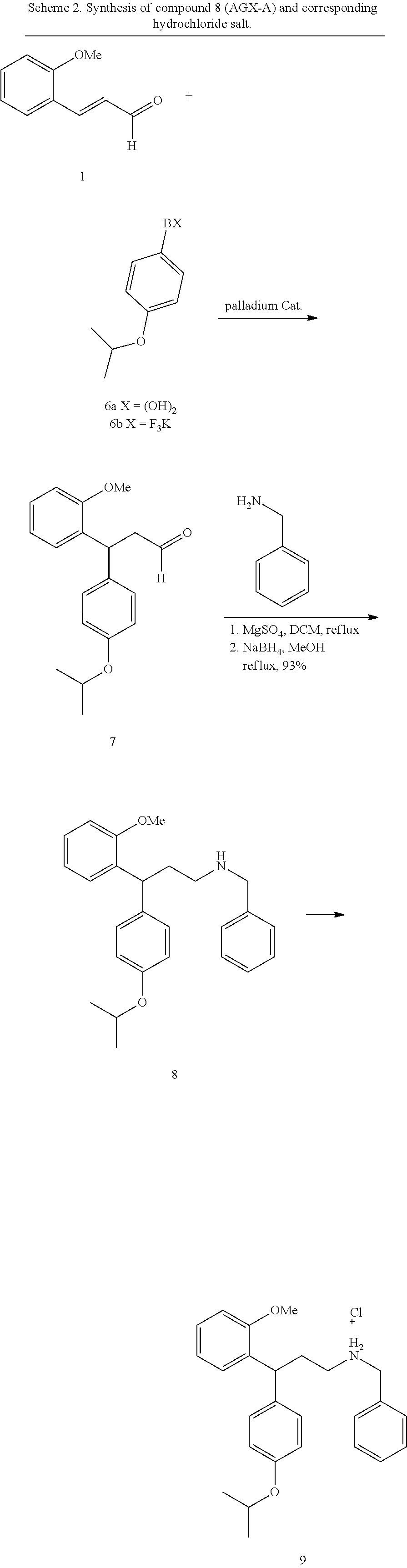

### Example 3. Reductive Amination Using Amines Baring Carbonyl Groups

The two-step reductive amination of aldehyde 3 (Scheme 3) with amines baring incompatibles groups such as ketones which could be reduced by reagents such as NaBH₄ were instead hydrogenated using hydrogen under palladium on charcoal catalysis. Compound 10 was obtained by hydrogenation of the intermediate imine using Pd 10% in charcoal under 1 Atmosphere hydrogen.

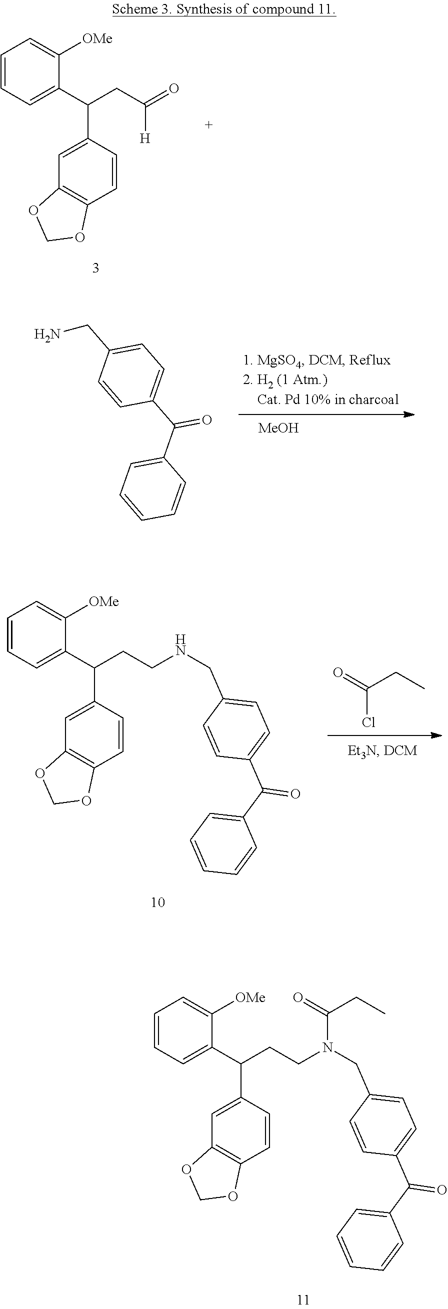

### Example 4. Chiral Resolution of AGX51

As indicated in Scheme 4, racemic AGX51 was resolved on a chiral AS-H prep column (Chiral technologies) to afford compound P1 (peak 1, >99% ee, [α]^{21.6}D +22.53 (c 0.8, MeOH)) and compound P2 (peak 2, ˜93% ee, [α]^{21.6}D −25.77 (c 0.8, MeOH)).

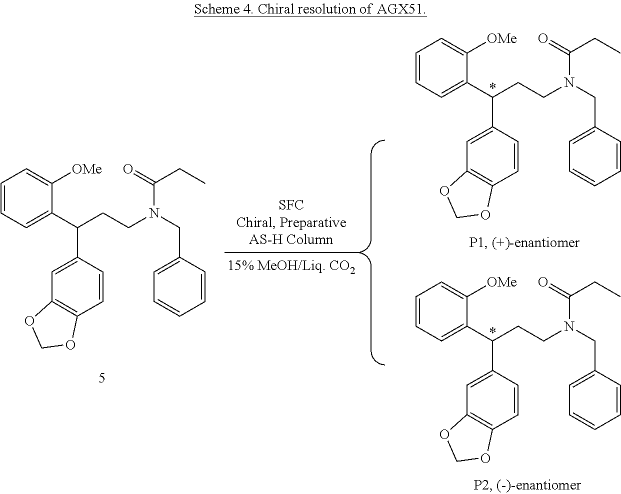 [SMILES: CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1]

### Example 5. Crystallization and X-Ray Structure Determination

Neither P1 nor P2 provided X-Ray quality crystals. However, because the corresponding amine salts were crystalline, the amide could be hydrolyzed under conditions, which should not compromise the chiral center. Hence, as indicated in Scheme 5, the (+)-enantiomer was submitted to 4.0 normal HCl in Dioxane-water at 85° C. for 24 hours which provided the corresponding amine after basic work up.

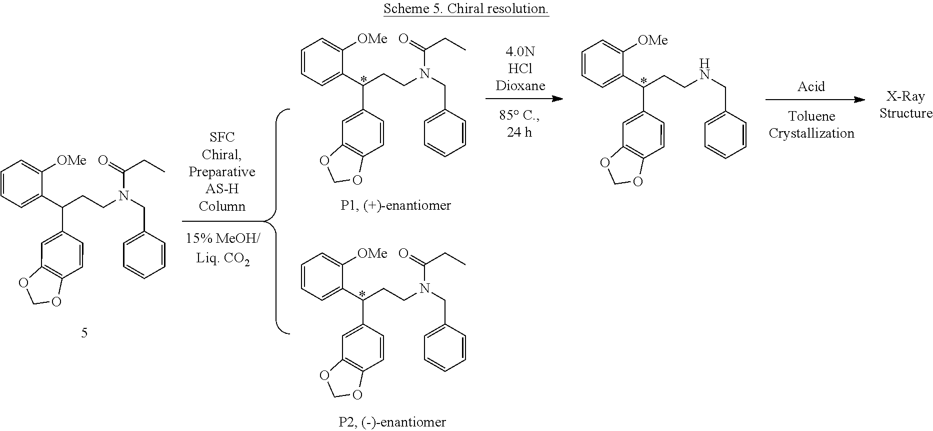 [SMILES: CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; CCC(=O)N(CCC(c1ccc2c(c1)OCO2)c1ccccc1OC)Cc1ccccc1 ; COc1ccccc1C(CCNCc1ccccc1)c1ccc2c(c1)OCO2]

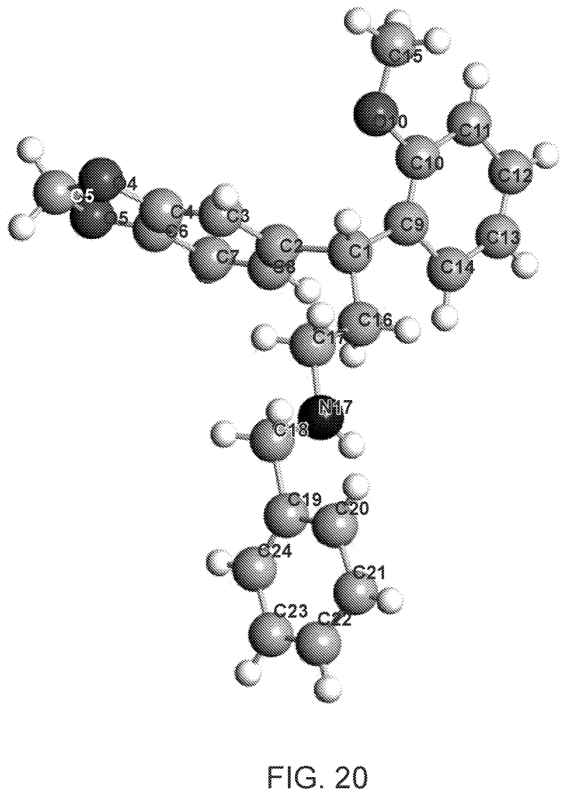

Thus obtained (+)-chiral amine was then crystallized with several acids; (+)-camphor sulfonic acid, D-(−)-tartaric acid, L-(+)-tartaric acid, L-(−)-malic acid, and fumaric acid in ethanol (toluene outer chamber). Crystals were harvested and submitted for X-Ray crystallography. Only crystals from the fumarate diffracted and provided unequivocally the conformation of the chiral center as the R-(+)-enantiomer. X-Ray structure of the P1, R-(+) enantiomer (i.e., R-(+)-3-(benzo[d][1,3]dioxol-5-yl)-N-benzyl-3-(2-methoxyphenyl)propan-1-amine) is shown in FIG. 20.

### Example 6. Deacylation of Both Enantiomers of AGX51

Deacylation of both optically pure amides P1 and P2 was additionally achieved by in situ activation of the inert tertiary amide followed by addition of phenyl Grignard reagent (Huang at al., Tetrahedron, 71(2015): 4248-4254), as indicated in Scheme 6. NMR analysis of both optical amines is identical to the one of synthesized amine 4 from Scheme 1.

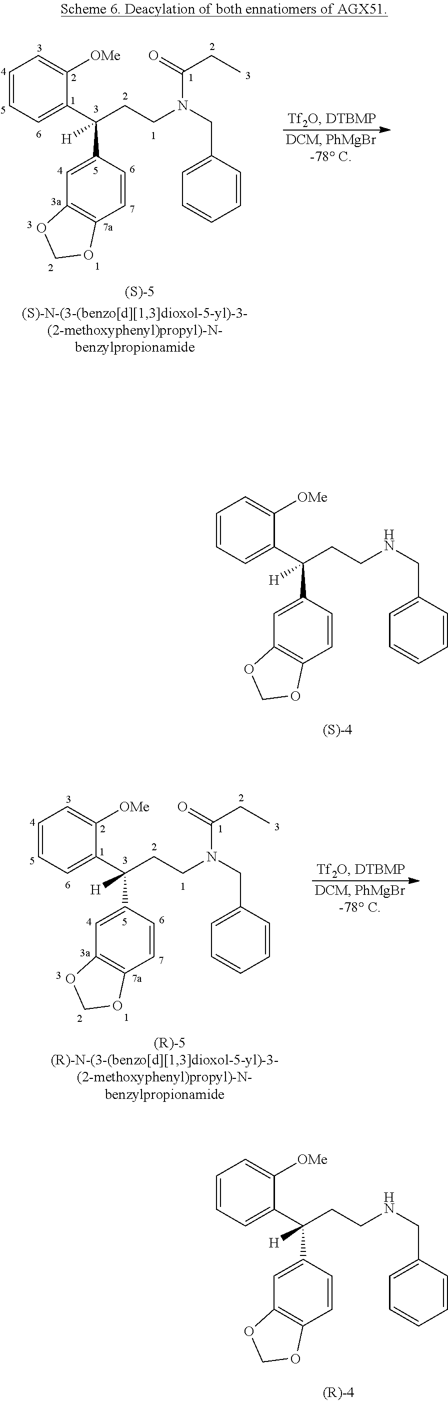 [SMILES: CCC(=O)N(CC[C@@H](c1ccc2c(c1)OCO2)c1ccccc1OC)Cc1ccccc1 ; COc1ccccc1[C@@H](CCNCc1ccccc1)c1ccc2c(c1)OCO2 ; CCC(=O)N(CC[C@H](c1ccc2c(c1)OCO2)c1ccccc1OC)Cc1ccccc1 ; COc1ccccc1[C@H](CCNCc1ccccc1)c1ccc2c(c1)OCO2]

### Example 7. Synthesis of Cross Linker Compound 16

Additionally, as indicated in Scheme 7, the direct chiral synthesis could be achieved through organo-catalysis using a chiral catalyst such as R—(C₇F₇)₂-BINOL (Angew. Chem. Int. Ed. Eng., 54(34): 9931-9935 (2015)). Indeed, reacting aldehyde 1 with trifluoroboronate 2a in the presence of catalytic amounts of R—(C₇F₇)₂-BINOL in toluene at 95° C. with molecular sieves, produced chiral aldehyde 3, which was advanced to the corresponding chiral amine 12. Following functional group manipulations to install the trifluoro acetamide and remove the Boc protecting group, reductive amination on the resulting amine using aldehyde 15 provided diazirine 16 which displayed a specific rotation of [α]²²D −70.39 (c 1.0, CH₂Cl₂). Enantiomeric excess was not determined. Aldehyde 15 was the product of a Dess-Martin periodinane oxidation of alcohol 14.

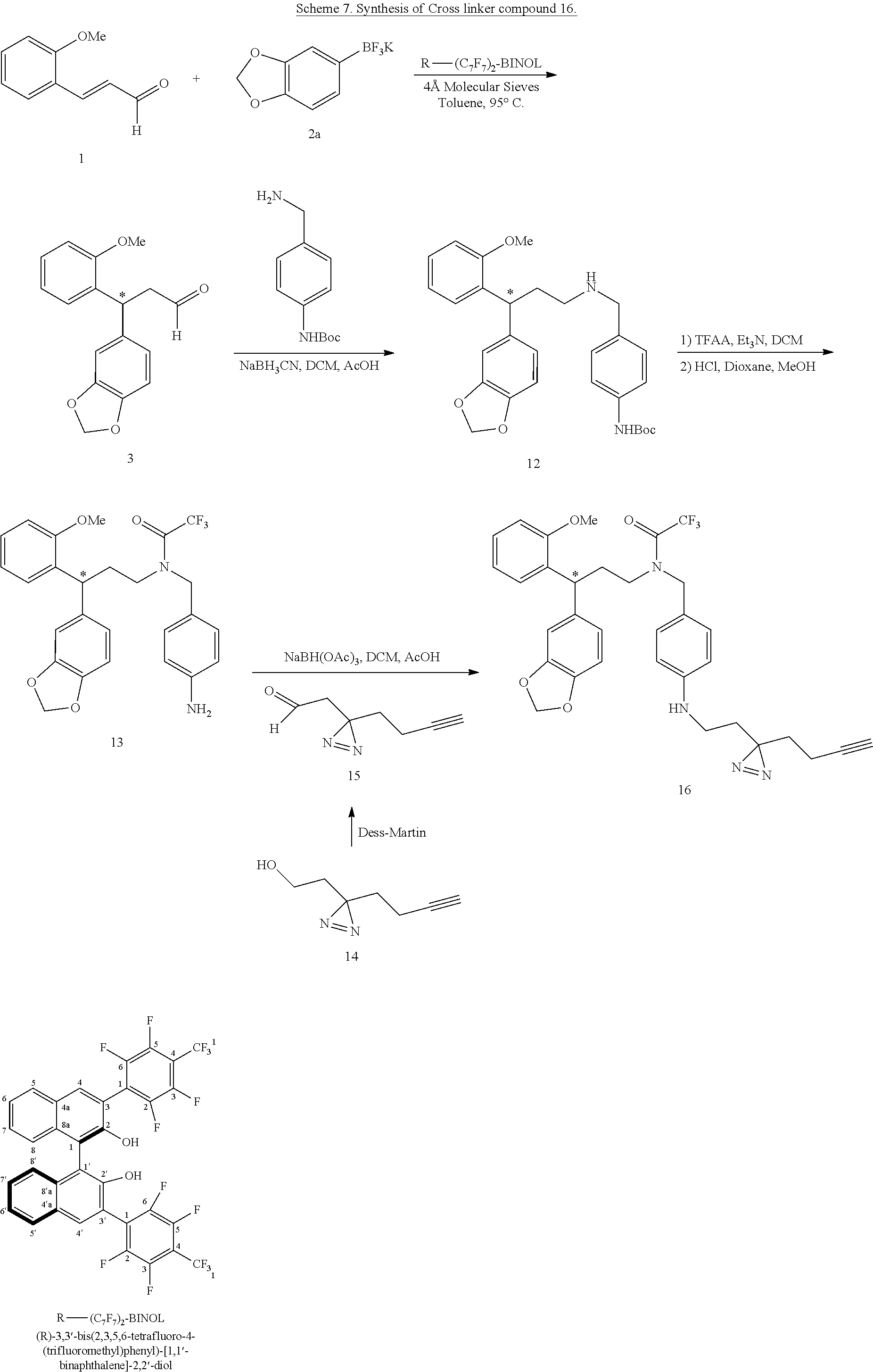

### Example 8. Synthesis of AGX-E

Additional analogs were modified through functional group manipulation, as exemplified in Scheme 8. The chloride in compound 17 (AGX-C) was substituted for an acetate using sodium acetate under microwave irradiation to lead to AGX-D (18) which in turn was hydrolyzed to bring in AGX-E (19) (Schema 6).

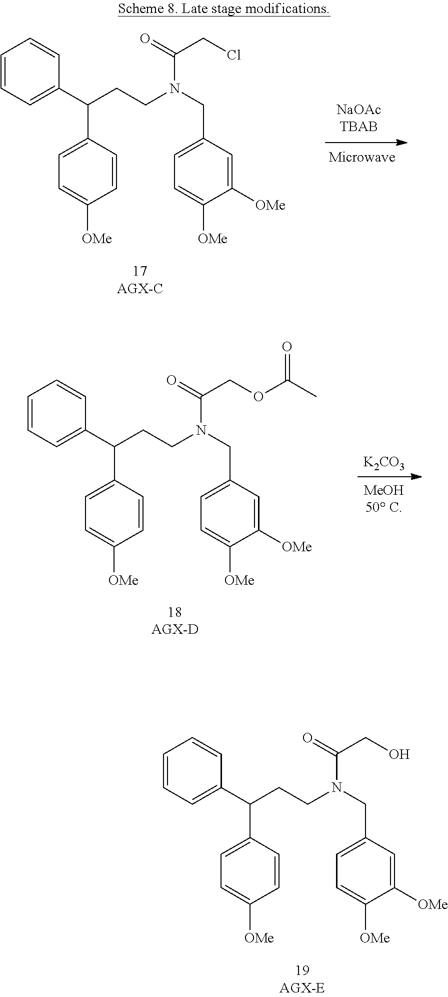 [SMILES: COc1ccc(C(CCN(Cc2ccc(OC)c(OC)c2)C(=O)CCl)c2ccccc2)cc1 ; COc1ccc(C(CCN(Cc2ccc(OC)c(OC)c2)C(=O)COC(C)=O)c2ccccc2)cc1 ; "K2CO3" ; COc1ccc(C(CCN(Cc2ccc(OC)c(OC)c2)C(=O)CO)c2ccccc2)cc1]

### Example 9. Synthesis of More AGX51 Analogs

As indicated in Scheme 9, compounds 21 (AGX-F) and 24 (AGX-H) were provided by amide formation of secondary amine 4 with the corresponding acids 20, 22 in good yield (Scheme 7).

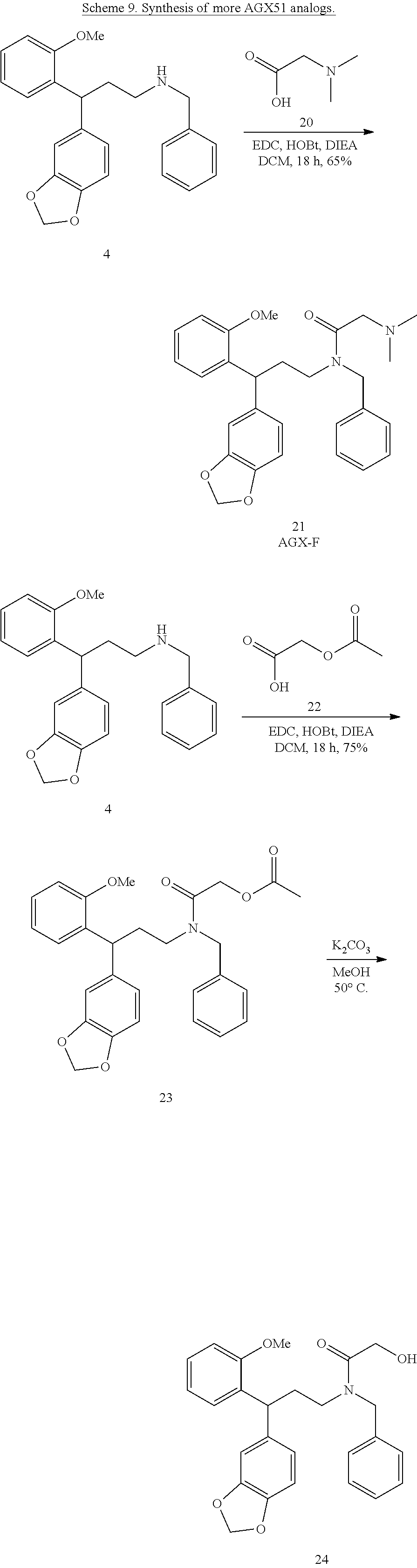

### Example 10. Synthesis of Cross Linker AGX51 Analogs

Several cross-linking as well as fluorescent probes were also synthesized, as indicated in Scheme 10. AGX51-BODIPY analogs can be made by coupling the amine 4 and PEG spacers 28, 31 with BODIPY NHS ester 25 (Scheme 7).

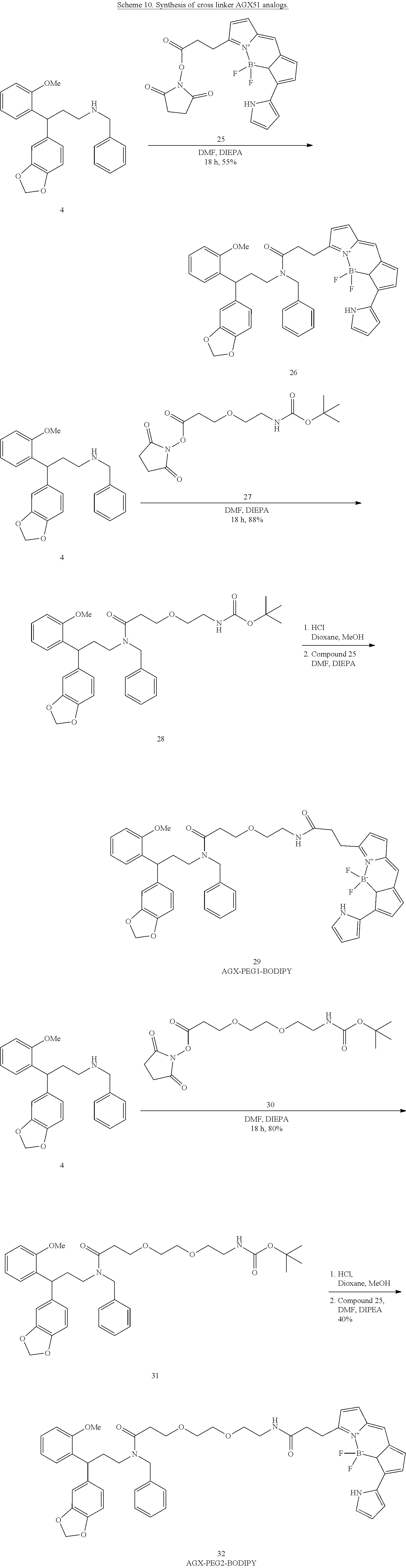

### Example 11. Experimental Procedures

Typical procedure A for conjugate addition of potassium organotrifluoroborate 2a to 2-methoxycinnamaldehyde. To a flask were added potassium organotrifluoroborate 2a (6.183 g, 27 mmol, 2.5 equ), 2-methoxycinnamaldehyde 1 (1.76 g, 10.85 mmol), Pd(OAc)₂ (122 mg, 0.54 mmol, 5 mol %), bpy (339 mg, 2.17 mmol, 20 mol %), HOAc (10 mL), THF (5.4 mL), and H₂O (3.2 mL) under Argon. The mixture was stirred and heated at 60° C. for 2-3 days. The reaction mixture was cooled to room temperature and filter and washed with EtOAc. The filtrate was neutralized with saturated NaHCO₃ and then extracted with ethyl acetate (×3). The combined organic layers was washed with saturated NaCl, dried (Na₂SO₄) and concentrated. The residue was purified by ISCO Combi Flash SiO₂ (12 g) column (20% ethyl acetate/cyclohexane) to give the product 3 (2.83 g) with 92% yield as a yellow oil.

Typical procedure B for conjugate addition of Arylboronic acid to 2-methoxycinnamaldehyde. To a flask were added arylboronic acid 6a (2.82 g, 15.67 mmol, 2.5 eq), 2-methoxycinnamaldehyde 1 (1 g, 6.17 mmol), Pd(OAc)₂ (69 mg, 0.308 mmol, 5 mol %), bpy (192 mg, 1.22 mmol, 20 mol %), HOAc (6 mL), THF (3 mL), and H₂O (1.8 mL) under Argon. The mixture was stirred and heated at 60° C. for 2-3 days. The reaction mixture was neutralized with saturated NaHCO₃ and then extracted with ethyl acetate (×3). The combined organic layers was washed with saturated NaCl, dried (Na₂SO₄) and concentrated. The residue was purified by ISCO Combi Flash SiO₂ (24 g) column (25% ethyl acetate/cyclohexane) to give the product 7 (1.46 g) with 80% yield as a yellow oil.

Genera/procedure C Reductive amination: To a solution of aldehyde 3 (7.66 g, 26.96 mmol) and benzyl amine (3.17 g, 29.66 mmol) in dichloromethane (150 mL) was added anhydrous magnesium sulfate (4.85 g, 40.44 mmol). After stirring for 1 h at reflux, the reaction mixture was filtered to remove the drying agent and the solvent was removed in vacuo. The crude imine was then dissolved in methanol (100 mL) and sodium borohydride (2.04 g, 53.92 mmol) was added while stirring at 0° C. After additional stirring for 1 h at reflux, the reaction mixture was quenched with water and concentrated to remove methanol. The residue was diluted with dichloromethane, and washed with water. The aqueous layer was extracted with dichloromethane (×3), the combined organic layer was washed saturated NaCl, dried (Na₂SO₄) and concentrated. The residue was purified by ISCO Combi Flash SiO₂ (120 g) column (5% MeOH/ethyl acetate) to give the product 5 (9.36 g) with 92% yield as a yellow oil.

3-(benzo[d][1,3]dioxol-5-yl)-3-(2-methoxyphenyl)propanal 3. ¹H NMR (CDCl, 600 MHz) δ 9.69 (t, J=2.2 Hz, 1H), 7.19 (dt, J=7.5, 1.6 Hz, 1H), 7.07 (dd, J=7.6, 1.5 Hz, 1H), 6.9 (t, J=7.5 Hz, 1H), 6.86 (d, J=8.2 Hz, 1H), 6.73 (m, 3H), 5.91 (s, 2H), 4.95 (t, J=7.9 Hz, 1H), 3.82 (s, 3H), 3.05 (dd, J=7.9, 2.2 Hz, 2H); ¹³C NMR (CDCl₃, 150 MHz) δ 201.75, 156.53, 147.71, 146.06, 136.75, 131.65, 127.88, 127.84, 120.88, 120.71, 110.78, 108.66, 108.14, 100.92, 55.40, 48.63, 37.96.

N-benzyl-3-(4-isopropoxyphenyl)-3-(2-methoxyphenyl)propan-1-amine 4 ¹H NMR (CDCl₃, 600 MHz) δ 7.30-7.19 (m, 6H), 7.15 (dt, J=8.1, 1.4 Hz, 1H), 6.89 (t, J=7.4 Hz, 1H), 6.81 (d, J=8.1 Hz, 1H), 6.73 (m, 2H), 6.68 (d, J=8.0 Hz, 1H), 5.86 (s, 2H), 4.41 (t, J=7.8 Hz, 1H), 3.76 (s, 3H), 3.72 (s, 2H), 2.60 (t, J=7.1 Hz, 2H), 2.17 (m, 2H); ¹³C NMR (CDCl₃, 150 MHz) δ 156.99, 147.58, 145.72, 140.67, 138.46, 133.46, 128.51, 128.23, 127.66, 127.32, 127.00, 121.10, 120.82, 110.84, 108.80, 108.10, 100.89, 55.61, 54.03, 47.95, 40.74, 35.45.

### N-(3-(benzo[d][1,3]dioxol-5-yl)-3-(2-methoxyphenyl)propyl)-N-benzylpropionamide 5 (AGX51)

General procedure D: To a solution of the amine 8 (6.14 g, 16.37 mmol) and triethylamine (4.56 mL, 32.75 mmol) in anhydrous dichloromethane (100 mL), propionyl chloride (3.57 mL, 40.92 mmol) was added dropwise at 0° C. After the reaction mixture was left overnight at room temperature, the resulting solution was poured into water and separated. The water layer was extracted with dichloromethane (×3). The combined organic layers were washed with saturated NaCl and then dried over (Na₂SO₄) and concentrated. The residue was purified by ISCO Combi Flash SiO₂ (120 g) column (30-40% ethyl acetate/hexanes) to give the product 10 (6.35 g) with 90% yield as a sticky yellow oil. The resulting clear sticky syrup was treated with ethanol (9 mL) and mixed well. After sitting in freezer overnight, white precipitate was formed, the white solid was filtered and washed with cold ethanol to afford dried white powder 5.77 g (AGX51). Mp: 87-88° C.

¹H NMR (DMSO-d₆, 600 MHz) δ 7.30-7.18 (m, 4H), 7.15 (m, 1H), 7.08 (m, 2H), 6.92-6.88 (m, 2H), 6.84-6.74 (m, 2H), 6.68 (m, 1H), 5.93 (m, 2H), 4.54-4.40 (m, 2H), 4.18 (t, J=7.9 Hz, 1H), 3.73, 3.72 (s, s, 3H), 3.16-2.98 (m, 2H), 2.54-2.05 (m, 4H), 0.95 (t, J=7.3 Hz, 3H); ¹³C NMR (CDCl₃, 150 MHz) δ 173.91, 173.83, 156.68, 147.66, 147.41, 145.91, 145.59, 138.34, 137.96, 137.47, 137.21, 132.79, 132.26, 128.80, 128.45, 128.24, 127.58, 127.44, 127.33, 127.19, 127.16, 126.34, 120.83, 120.81, 120.72, 120.68, 110.72, 110.66, 108.56, 108.48, 108.11, 107.95, 100.87, 100.70, 55.41, 55.37, 51.48, 48.05, 45.99, 45.36, 40.90, 40.50, 33.61, 32.54, 26.55, 26.02, 9.67, 9.48; high res MS calc. for C₂₇H₃₀NO₂₄ 432.2175, found 432.2191 (M+H).

3-(4-isopropoxyphenyl)-3-(2-methoxyphenyl)propanal 7. ¹H NMR (CDCl₃, 600 MHz) δ 9.69 (t, J=2.3 Hz, 1H), 7.18 (dt, 1H, J=8.1, 1.7 Hz), 7.14 (m, 2H), 7.05 (dd, J=7.6, 1.6 Hz, 1H), 6.88 (dt, J=7.5, 0.9 Hz, 1H), 6.85 (d, J=8.2, 0.9 Hz, 1H), 6.80 (m, 2H), 4.96 (t, J=7.9 Hz, 1H), 4.49 (m, 1H), 3.81 (s, 3H), 3.05 (dd, J=7.9, 2.3 Hz, 2H), 1.31 (d, J=6.0 Hz, 6H); ¹³C NMR (CDCl₃, 150 MHz) δ 202.15, 156.57, 156.43, 134.55, 132.05, 129.03, 128.08, 127.68, 120.67, 115.72, 110.73, 69.79, 55.39, 48.63, 37.53, 22.10.

N-benzyl-3-(4-isopropoxyphenyl)-3-(2-methoxyphenyl)propan-1-anine 8 (AGX-A). ¹H NMR (CDCl₃, 600 MHz) δ 7.29-7.18 (m, 6H), 7.16-7.12 (m, 3H), 6.88 (dt, J=7.5, 0.9 Hz, 1H), 6.81 (d, J=8.2 Hz, 1H), 6.75 (m, 2H), 4.48-4.41 (m, 2H), 3.75 (s, 3H), 3.71 (s, 2H), 2.60 (t, J=7.2 Hz, 2H), 2.19 (m, 2H), 1.29 (d, J=6.1 Hz, 6H); ¹³C NMR (CDCl₃, 150 MHz) δ 156.90, 156.04, 140.54, 136.62, 133.71, 129.02, 128.09, 127.72, 127.00, 126.83, 120.64, 115.53, 110.68, 69.77, 55.47, 53.88, 47.91, 40.09, 35.29, 30.39, 22.18; high res MS calc. for C₂₆H₃₂NO₂ 390.2433, found 390.2426 (M+H).

AGX-A Hydrogen chloride salt 9 formation: Benzyl amine AGX-A 8 (9.92 g, 25.5 mmol) in dichloromethane (40 mL) was treated with HCl (2 N in ether, 14 mL, 28 mmol) in ice bath. The mixture was stirred for 2 h. After removal of solvent, the sticky syrup was treated with hexane twice and concentrated in vacuo. The syrup (5 g) was titrated with ethyl acetate (10 mL) to form white precipitate. The white solid was filtered and washed with hexane to afford AGX-A HCl 9 salt (10.6 g) as a white powder. Mp: 158-160° C.

(4-(((3-(benzo[d][1,3]dioxol-5-yl)-3-(2-methoxyphenyl)propyl)amino)methyl)phenyl) (phenyl)methanone 10. 4-(Aminomethyl)phenyl](phenyl)methanone hydrochloride (66 mg, 0.27 mmol) was pretreated with K₂CO₃ (5 equiv) in dichloromethane (10 mL) and water (2 mL). After stirring at room temperature for 2 h, the aqueous layer was extracted with dichloromethane (×3), the combined organic layer was washed with saturated NaCl and then dried over (Na₂SO₄) and concentrated to afford the colorless oil directly used for the next step.

The resulting free amine was treated with aldehyde 3 using the general procedure C to afford the imine, which was treated with 10% Pd/C (48 mg) in MeOH (4 mL) and dichloromethane (2 mL) with a hydrogen balloon for 2 h. The reaction mixture was filtered through celite and washed with mixture of MeOH and dichloromethane. After removal of the solvents, the residue was purified by ISCO Combi Flash SiO₂ (4 g) column (5% MeOH/dichloromethane) to give the product 10 (60 mg) with 50% yield as a foaming solid. ¹H NMR (CDCl₃, 500 MHz) δ 7.75 (m, 4H), 7.59 (t, J=7.4 Hz, 1H), 7.53 (d, J=7.9 Hz, 2H), 7.46 (t, J=7.6 Hz, 2H), 7.13 (m, 2H), 6.86 (t, J=7.4 Hz, 1H), 6.78 (d, J=8.1 Hz, 1H), 6.70 (m, 2H), 6.63 (d, J=8.3 Hz, 1H), 5.83 (s, 2H), 4.36 (t, J=7.5 Hz, 1H), 3.97 (s, 2H), 3.75 (s, 3H), 2.71 (t, J=7.3 Hz, 2H), 2.41 (m, 2H); ¹³C NMR (CDCl₃, 125 MHz) δ 195.06, 180.18, 170.20, 155.69, 146.56, 144.86, 136.57, 136.34, 136.29, 131.63, 130.96, 129.52, 129.04, 128.27, 127.37, 126.58, 126.48, 119.85, 119.81, 109.76, 107.51, 107.06, 99.82, 59.43, 54.49, 39.57, 20.08, 13.22.

N-(3-(benzo[d][1,3]dioxol-5-yl)-3-(2-methoxyphenyl)propy)-N-(4-benzoylbenzyl) propionamide 13. The above amine 10 was treated with propionyl chloride (29 μL, 0.313 mmol) followed with the general procedure D to afford the compound 11 (66 mg, 98%) as a sticky syrup. ¹H NMR (CDCl₃, 600 MHz) δ 7.81-7.70 (m, 4H), 7.60 (m, 1H), 7.49 (m, 2H), 7.25-7.12 (m, 4H), 6.91 (m, 1H), 6.85-6.79 (m, 1H), 6.75-6.67 (m, 3H), 5.88-5.84 (m, 2H), 4.69-4.51 (m, 2H), 4.31-4.23 (t, t, J=7.9, 7.9 Hz, 1H), 3.78, 3.75 (s, s, 3H), 3.34, 3.17 (t, t, J=7.6, 7.6 Hz, 2H), 2.30-2.22 (m, 4H), 1.12 (t, J=7.4 Hz, 3H); ¹³C NMR (CDCl₃, 150 MHz) δ 196.35, 196.21, 174.07, 173.93, 156.68, 147.71, 147.48, 145.99, 145.67, 142.84, 142.05, 138.21, 137.64, 137.45, 137.35, 136.91, 136.54, 132.69, 132.56, 132.14, 130.68, 130.38, 130.02, 130.00, 128.36, 128.29, 127.87, 127.69, 127.31, 127.13, 126.21, 120.80, 120.78, 120.72, 110.78, 110.72, 108.53, 108.44, 108.16, 108.00, 100.92, 100.75, 60.41, 55.45, 55.42, 51.36, 48.12, 46.14, 45.81, 40.90, 40.47, 33.71, 32.58, 26.60, 26.02, 21.07, 9.66, 9.47.

Preparation of Chiral AGX51 using Chiral Column. Racemic AGX51 5 (1 g) was resolved by waters company using chiral AS-H prep column eluting with 15% MeOH/liq CO₂ 3.5 ml/min to get AGX51 P1 (99% ee, 460 mg syrup with [α]=22.53 (c=0.8 MeOH). AGX51 P2 (93% ee, 480 mg syrup with [α]=−25.77 (c=0.8 MeOH).

General procedure for the direct transformation of chiral tertiary amide into chiral amine. Tf₂O (1.2 equiv) was added dropwise to a cooled (−78° C.) solution of amide AGX51 (P1) and 2,6-Di-tert-butyl-4-methylpyridine (DTBMP 1.2 equiv) in CH₂C₁₂ (5 mL). The reaction was allowed warming to 0° C. over 2 h. A solution of Grignard reagent PhMaBr (1 N THF, 1.0 equiv) was added dropwise to the resultant mixture at −78° C., and the mixture was stirred at the same temperature for 2 h. The reaction mixture was then quenched with an aqueous solution of NH₄Cl. The organic layer was separated and the aqueous layer was extracted with ethyl acetate (×3). The combined organic layers were washed with brine, dried over (Na₂SO₄) and concentrated. The residue was purified by ISCO Combi Flash SiO₂ column (50-70% ethyl acetate/hexanes) to give the chiral product 4.

Amine 4P1 was afforded from AGX51 (P1), [α]²¹D 39.66 (c 1.7, MeOH). Amine 4P2 was afforded from AGX51 (P2), [α]²²D −38.49 (c 1.3, MeOH).

3-(benzo[d][1,3]dioxol-5-yl)-3-(2-methoxyphenyl)propanal (3). To a flask equipped with a stir bar was added powdered 4 Å molecular sieves (0.7 g) and the flask was flame-dried under vacuum. The flask was cooled to rt under Argon. The aldehyde 1 (100 mg, 0.616 mmol), ligand R—(C₇F₇)₂BINOL (88 mg, 0.12 mol, 0.2 equ), potassium aryl trifluoroborate salt 2a (422 mg, 1.85 mmo, 3 equ) were added followed by anhydrous toluene (12 mL). The reaction was heated at 95° C. for 3 days. The reaction mixture was then filtered through celite and washed with ether. After concentration the residue was purified on 4 g silica gel column eluting with 5-10% acetone/hexane to afford product 3 (70 mg, 40%) and recover starting material aldehyde 1. ¹H NMR (CDCl₃, 600 MHz) δ 9.69 (t, J=2.2 Hz, 1H), 7.19 (dt, J=7.5, 1.6 hz, 1H), 7.07 (dd, J=7.6, 1.5 Hz, 1H), 6.9 (t, J=7.5 Hz, 1H), 6.86 (d, J=8.2 Hz, 1H), 6.73 (m, 3H), 5.91 (s, 2H), 4.95 (t, J=7.9 Hz, 1H), 3.82 (s, 3H), 3.05 (dd, J=7.9, 2.2 Hz, 2H); ¹³C NMR (CDCl₃, 150 MHz) δ 201.75, 156.53, 147.71, 146.06, 136.75, 131.65, 127.88, 127.84, 120.88, 120.71, 110.78, 108.66, 108.14, 100.92, 55.40, 48.63, 37.96. [α]²²D −70.39 (c 1.0, CH₂Cl₂).

### tert-butyl (4-(((3-(benzo[d][1,3]dioxol-5-yl)-3-(2-methoxyphenyl)propyl)amino)methyl) phenyl)carbamate 12

General procedure. A mixture of aldehyde 3 (20 mg, 0.07 mmol), tert-butyl (4-aminomethyl)phenyl)carbamate (18 mg, 0.081 mmol) and acetic acid (5 μL, 0.086 mmol) in ClCH₂CH₂Cl (1 ml) was stirred at rt for 30 min before adding sodium triacetoxyborohydride (37 mg, 0.175 mmol). The resulting mixture was stirred at rt for 18 h. The mixture was concentrated and dissolved in MeOH and purified on 4 g silica gel column to give product 12 (25 mg, 60%) as a colorless oil. ¹H NMR (CDCl₃, 600 MHz) δ 7.26 (m, 1H), 7.19-7.13 (m, 4H), 6.89 (t, J=7.5 Hz, 1H), 6.81 (d, J=8.0 Hz, 1H), 6.71-6.66 (m, 3H), 6.55 (brs, 1H), 6.23 (br, 2H), 5.87 (s, 2H), 4.34 (t, J=7.9 Hz, 1H), 3.75 (s, 3H), 3.73 (s, 2H), 2.61 (m, 2H), 2.23 (m, 2H), 1.51 (s, 9H); ¹³C NMR (CDCl₃, 150 MHz) δ 156.73, 147.49, 145.70, 138.05, 137.87, 132.68, 129.46, 127.75, 120.89, 120.75, 118.56, 110.71, 108.57, 108.00, 100.76, 80.57, 55.45, 51.79, 46.38, 40.57, 33.44, 28.35; [α]²²D −30.45 (c 1.25, MeOH); high res MS calc. for C₂₉H₃₅N₂O₅ 491.2546, found 491.2534 (M+H).

N-(4-aminobenzyl)-N-(3-(benzo[d][1,3]dioxol-5-yl)-3-(2-methoxyphenyl)propyl)-2,2,2-trifluoroacetamide 13. A solution of amine 12 (25 mg, 0.051 mmol) and triethylamine (21 μl, 0.153 mmol) in CH₂Cl₂ (1 mL) was treated with trifluoroacetic anhydride (18 μL, 0.127 mmol at room temperature. After the reaction mixture was stirred for 4 h, the solution was concentrated and purified on 4 g silica gel column to give product (18 ng, 60%). ¹H NMR (CDCl₃, 600 MHz) δ 7.30 (m, 1H), 7.21-7.15 (m, 1H), 7.10 (t, J=8.9 Hz, 1H), 6.92 (dd, J=8.3, 16.7 Hz, 2H), 6.92-6.80 (m, 2H), 6.72-6.67 (m, 3H), 6.48 (d, J=19.1 Hz, 1H), 5.89 (m, 2H), 4.51 (s, 1H), 4.46 (s, 1H), 4.22 (m, 1H), 3.78, 3.76 (s, s, 3H), 3.22 (m, 2H), 2.28-2.14 (m, 2H), 1.51 (s, 9H); ¹⁹F decH δ −67.96, −68.96; [α]²²D −24.02 (c 0.85, MeOH); ES MS calc. for C₃₁H₃₃FN₂O₆ 586.23, found 609.3 (M+Na).

The intermediate prepared above (17 ng, 0.029 mmol) was dissolved in a dioxane/HCl solution (4 N, 0.2 mL) and MeOH (1 mL) and stirred at room temperature for 3 hours. The reaction mixture was concentrated and the residue free-based with a concentrated aqueous NaHCO₃ solution. This material was purified by chromatography on silica eluting with 5% MeOH in CH₂C₂ to give the product 13 (13 ng, 92%). ¹H NMR (CDCl₃, 600 MHz) δ 7.19-7.15 (m, 1H), 7.10 (m, 1H), 6.93-6.80 (m, 4H), 6.72-6.66 (m, 3H), 6.61 (d, J=8.4 Hz, 1H), 6.58 (d, J=8.3 Hz, 1H), 5.89 (dd, J=4.9, 12.9 Hz, 2H), 4.46 (s, 1H), 4.39 (s, 1H), 3.78, 3.76 (s, s, 3H), 3.20 (m, 2H), 2.29-2.10 (m, 2H);); ¹⁹F decH δ −67.82, −68.94; [α]²²D −29.16 (c 0.55, MeOH); ES MS calc. for C₂₆H₂₅N₂O₄ 486.18, found 509.3 (M+Na).

N-(3-(benzo[d][1,3]dioxol-5-yl)-3-(2-methoxyphenyl)propyl)-N-(4-((2-(3-(but-3-yn-1-yl)-3H-diazirin-3-yl)ethyl)amino)benzyl)-2,2,2-trifluoroacetamide 16. Compound 16 can be synthesized using the general procedure by treatment of amine 13 (13 mg, 0.0267 mmol) and aldehyde 15 (5 mg, 0.04 mmol). ¹H NMR (CDCl₃, 600 MHz) δ 7.19-7.15 (m, 1H), 7.10 (m, 1H), 6.93-6.80 (m, 4H), 6.72-6.66 (m, 3H), 6.51 (d, J=8.5 Hz, 1H), 6.47 (d, J=8.5 Hz, 1H), 5.89 (m, 2H), 4.46 (s, 1H), 4.22 (s, 1H), 3.78, 3.76 (s, s, 3H), 3.20 (m, 2H), 2.92 (m, 2H), 2.25-2.04 (m, 2H), 2.01 (m, 3H), 1.80 (m, 2H), 1.67 (m, 2H); ¹⁹F decH δ −67.79, −68.94; ES MS calc. for C₃₃H₃₃F₃N₂₄O₄ 606.25, found 607.3 (M+H).

2-((3,4-dimethoxybenzyl)(3-(4-methoxyphenyl)-3-phenylpropyl)amino)-2-oxoethyl acetate (22, AGX-D). To a mixture of GY-AGX-C 17 (47 mg, 1 mmol) in benzene (1 mL) in a microwave tube, NaOAc (52 mg, 6 mmol) was added followed by Tetrabutyl ammonium bromide (TBABr, 6 mg, 0.02 mmol). The reaction was heated under microwave at 120° C. for 30 min. The reaction mixture was diluted with Ethyl acetate and washed with water. The organic layer was dried over anhydrous Na₂SO₄ and concentrated. The residue was purified on silica (4 g column) eluting with 50% EA/hexanes to give the product 18 (16 mg, 93%). ¹H NMR (CDCl₃, 600 MHz) δ 7.28-7.25 (m, 2H), 7.17-7.11 (m, 3H), 7.08 (m, 2H), 6.83-6.63 (m, 5H), 4.69, 4.51 (s, s, 2H), 4.45, 4.27 (s, s, 2H), 3.88-3.74 (m, 9H), 3.30, 3.05 (m, 2H), 2.29 (m, 2H), 2.17 (s, 3H); ES MS calc. for C₂₉H₃₃NO₆ 491.23, found 492.3 (M+H).

N-(3,4-dimethoxybenzyl)-2-hydroxy-N-(3-(4-methoxyphenyl)-3-phenylpropyl)acetamide (19, AGX-E). The reaction mixture of acetate 18 (42 mg, 0.085 mmol) in MeOH (1 mL) was added K₂CO₃ (38 mg, 0.26 mmol) and heated at 50° C. for 2 h. The mixture was filtered and washed with DCM, the solvents was concentrated. The resulting residue was purified on silica (4 g column) eluting with 50% EA/hexanes to give the product 19 (32 mg, 86%). ¹H NMR (CDCl₃, 600 MHz) δ 7.29-7.24 (m, 2H), 7.20-7.16 (m, 3H), 7.14-7.07 (m, 2H), 6.84-6.74 (m, 3H), 6.70-6.50 (m, 2H), 4.55 (s, 1H), 4.15 (s, 1H), 3.92 (d, J=4.1 Hz, 1H), 3.86-3.71 (m, 9H), 3.65, 3.61 (m, 1H), 3.35 (m, 1H), 2.95 (m, 1H), 2.31-2.21 (m, 2H); ES MS calc. for C₂₇H₃₁NO₅ 449.22, found 450.3 (M+H).

Classical amide formation method. Amine 4 (68 mg, 0.18 mmol) and carboxylic acid (1.2 equ) in DCM (2 mL) was treated with EDC (1.3 equ, 45 mg) and HOBt (1.3 equ, 32 mg) followed by DIEPA (4 equ, 126 μL). The reaction mixture was stirred at rt overnight and diluted with DCM, washed with sat. NaHCO₃. The organic layer was dried over anhydrous Na₂SO₄ and concentrated. The residue was purified on silica (4 g column) eluting with 5% MeOH/DCM to give the product 21 (53 mg, 65%), 23 (75%).

N-(3-(benzo[d][1,3]dioxol-5-yl)-3-(2-methoxyphenyl)propy)-N-benzyl-2-(dimethylamino)acetamide (21, AGX-F). ¹H NMR (CDCl₃, 600 MHz) δ 7.35-7.03 (m, 7H), 6.94-6.79 (m, 2H), 6.73-6.66 (m, 3H), 5.86 (m, 2H), 4.65-4.35 (m, 2H), 4.19 (m, 1H), 3.94-3.54 (m, 5H), 3.35, 3.05 (m, 2H), 2.91-2.86 (m, 6H), 2.16 (m, 2H); ES MS calc. for C₂₈H₃₂N₂O₄ 460.24, found 461.2 (M+H).

2-((3-(benzo[d][1,3]dioxol-5-yl)-3-(2-methoxyphenyl)propy)(benzyl)amino)-2-oxoethyl acetate (23, AGX-G). ¹H NMR (CDCl₃, 600 MHz) δ 7.35-7.24 (m, 3H), 7.20-7.09 (m, 4H), 6.91-6.78 (m, 2H), 6.67 (m, 3H), 5.90 (2H), 4.66-4.55 (m, 2H), 4.51, 4.36 (s, s, 2H), 4.22 (m, 1H), 3.78, 3.75 (s, s, 3H), 3.33, 3.06 (m, m, 2H), 2.22 (m, 2H), 2.19, 2.12 (s, s, 3H); ES MS calc. for C₂₈H₂₉NO₆ 475.20, found 476.2 (M+H).

N-(3-(benzo[d][1,3]dioxol-5-yl)-3-(2-methoxyphenyl)propyl)-N-benzyl-2-hydroxyacetamide (24, AGX-H). Compound 24 can be afforded using the same method of making compound 19. ¹H NMR (CDCl₃, 600 MHz) δ 7.31-7.25 (m, 3H), 7.18-7.03 (m, 4H), 6.90 (m, 1H), 6.84 (m, 1), 6.71-6.65 (m, 3H), 5.89-5.85 (m, 2H), 4.63 (s, 1H), 4.30-4.14 (m, 3H), 3.99 (s, 2H), 3.78, 3.75 (s, s, 3H), 3.36, 2.96 (m, t, 2H), 2.24-2.14 (m, 2H); ES MS calc. for C₂₆H₂₇NO₅ 433.19, found 434.2 (M+H).

General coupling reaction of amine 4 with NHS ester 25: To the solution of amine 4 (1 equ) and NHS ester 25 (1 eq) in DMF (1 mL) was added DIEPA (3 eq). The reaction mixture was stirred at rt overnight and subjected for purification.

Hydrolysis of NBoc reaction: AGX-PEG analogs 28, 31 in MeOH (1 mL) was treated with 4 N HCl (in dioxane, 10 equ). The reaction was stirred at rt for 4 h, then concentrated and dried under vacuum. Each HCl salt was used directly to the next step without purification.

HPLC Purification of AGX-BODIPY analogs: The solution of crude BODIPY mixture in MeOH and water was injected onto HPLC XBridge™ Prep C₁₈ column (5 μm, 19×150 mm) eluting with 60-90% ACN/Water (0.05% TFA in each solution) for 10 min, flow rate 20 ml/min collecting product by mass.

tert-Butyl (2-(3-((3-(benzo[d][1,3]dioxol-5-yl)-3-(2-methoxyphenyl)propyl)(benzyl) amino)-3-oxopropoxy)ethyl)carbamate 28. ¹H NMR (CDCl₃, 600 MHz) δ 7.33-7.24 (m, 3H), 7.20-7.08 (m, 4H), 6.92-6.79 (m, 2H), 6.70 (m, 3H), 5.90-5.86 (m, 2H), 5.07, 4.98 (brs, 1H), 4.57, 4.45 (s, s, 2H), 4.26 (m, 1H), 3.81, 3.74 (s, s, 3H), 3.73 (m, 2H), 3.48 (m, 2H), 3.33-3.13 (m, 4H), 2.56, 2.43 (t, t, 2H), 2.20 (m, 2H), 1.43, 1.41 (s, s, 9H); ES MS calc. for C₃₄H₄₂N₂O₇ 590.30, found 591.3 (M+H).

Tert-Butyl (2-(2-(3-((3-(benzo[d][1,3]dioxol-5-yl)-3-(2-methoxyphenyl)propyl)(benzyl) amino)-3-oxopropoxy)ethoxy)ethyl)carbamate 31. ¹H NMR (CDCl₃, 600 MHz) δ 7.32-7.22 (m, 3H), 7.21-7.07 (m, 4H), 6.92-6.79 (m, 2H), 6.69 (m, 3H), 5.90-5.86 (m, 2H), 5.05, 4.98 (brs, 1H), 4.56, 4.46 (s, s, 2H), 4.29-4.11 (m, 1H), 3.80 (m, 2H), 3.78, 3.75 (s, s, 3H), 3.58-3.50 (m, 6H), 3.33-3.14 (m, 4H), 2.60, 2.52 (t, t, 2H), 2.15 (m, 2H), 1.42 (s, 9H); ES MS calc. for C₃₆H₄₆N₂O₈ 634.33, found 635.3 (M+H).

AGX-BODIPY 26. ¹H NMR (CDCl₃, 600 MHz) δ 10.35 (brs, 1H), 7.28 (m, 2H), 7.17-7.02 (m, 6H), 6.97 (m, 2H), 6.91-6.58 (m, 7H), 6.36 (m, 1H), 6.21 (m, 1H), 5.88 (m, 2H), 4.59-4.42 (m, 1H), 4.29-4.15 (m, 1H), 3.75, 3.68 (s, s, 3H), 3.37-3.11 (m, 4H), 2.78-2.63 (m, 2H), 2.23-2.08 (m, 2H); ¹⁹F NMR (CDCl₃, 600 MHz) δ −75.67 (TFA), −140.09 (m, B—F); ES MS calc. for C₄₀H₃₇BF₂N₄O₄ 686.29, found 687.3 (M+H).

AGX-PEG1-BODIPY 29. ¹H NMR (CDCl₃, 600 MHz) δ 10.38 (brs, 1H), 7.21-7.16 (m, 2H), 7.12-7.04 (m, 3H), 7.02-6.92 (m, 5H), 6.90-6.77 (m, 4H), 6.66 (m, 3H), 6.35 (m, 1H), 6.26 (m, 1H), 5.86 (m, 2H), 4.51, 4.37 (s, s, 2H), 4.20 (m, 1H), 3.74 (s, 3H), 3.66 (m, 2H), 3.47-3.40 (m, 4H), 3.32-3.10 (m, 4H), 2.70 (m, 2H), 2.49, 2.40 (t, t, 2H), 2.11 (m, 2H); ¹⁹F NMR (CDCl₃, 600 MHz) δ −75.84 (TFA), −140.08 (m, B—F); ES MS calc. for C₄₅H₄₆BF₂N₅O₆ 801.35, found 802.3 (M+H).

AGX-PEG2-BODIPY 32. ¹H NMR (CDCl₃, 600 MHz) δ 10.38 (brs, 1H), 7.24-6.95 (m, 9H), 6.88-6.78 (m, 3H), 6.67 (m, 3H), 6.56 (m, 1H), 6.35-6.26 (m, 2H), 5.88 (m, 2H), 4.51, 4.37 (s, s, 2H), 4.25 (m, 1H), 3.73 (m, 4H), 3.51 (m, 5H), 3.41 (m, 2H), 3.33-3.08 (m, 4H), 2.66-2.43 (m, 4H), 2.16 (m, 2H), ¹⁹F NMR (CDCl₃, 600 MHz) δ −75.76 (TFA), −140.06 (m, B—F); ES MS calc. for C₄₇H₅₀BF₂N₅O₇ 845.38, found 846.3 (M+H).

### Example 12. Experimental Model and Subject Details

In vivo animal studies. Animal studies were carried out in accordance with institutional regulations (MO16M130 for the CNV neovascularization studies and M016M138 for the ROP studies) in a non-blinded fashion.

Pharmacokinetic analyses. To determine the pharmacokinetic parameters of AGX51, eight-week old male Balb/c mice (Taconic farms) were dosed once by i.p. injection with 30 mg/kg, 50 mg/kg or 100 mg/kg AGX51 prepared in 70% DMSO (n=3 mice per group). Another set of three mice was dosed with 100 mg/kg AGX51 prepared in 100% DMSO. Blood was collected at 30 minutes, 1 hour, 3 hours, 6 hours and 24 hours post AGX51 administration and plasma analyzed via LC-MS as per the protocol previously validated in the MSKCC Antitumor Assessment Core Facility. Following blood collection, the mice were sacrificed via CO₂ asphyxiation and the eyes from the 30 mg/kg treatment group were collected and flash frozen for analysis. Data obtained from LC-MS was analyzed via WinNonLin software (version 8.1) for pharmacokinetic parameters.

Toxicity analyses. To assess for toxicity, female athymic nude mice (Envigo), age 6-8 weeks, were dosed i.p. with either a control vehicle (70% DMSO in water) or AGX51 at 60 mg/kg twice daily for 14 consecutive days. Mice were sacrificed 24 hours after the last test administration. Gross and complete necropsy, along with clinical pathology analysis was conducted on all mice. Clinical Chemistry parameters measured were: BUN, Creatine, ALP, ALT, AST, GGT, Bilirubin, Total Protein, Albumin, Globulin, Phosphorus, Glucose, Cholesterol, Phosphorus, Calcium, Sodium, Potassium, Chloride. Hematology parameters measured were: white blood cells (lymphocytes, Monocytes, Eosinophils, Basophils, Neutrophils), red blood cells, Hemoglobin, Hematocrit, MCV, MCH, MCHC, RDW, Platelets. Organs/Tissues analyzed were: Lung, heart, thymus, kidneys, liver, spleen, gall bladder, pancreas, duodenum, jejunum, ileum, cecum, colon, bone marrow, femur, tibia, sternum, brain, eyes, ears, nasal and oral cavities, teeth, mesenteric and tracheal lymph nodes.

Mouse mode/s of ocular neovascularization. CNV was induced as previously described. Tobe et al., Am. J. Pathol., 153, 1641-1646 (1998). Briefly, 4-6 week-old female Id1^{-/-} or Id3^{-/-} mice and littermate control Id1^{+/+} or Id3^{+/+} mice (all in C₅₇BL/6 background) had laser-induced rupture of Bruch's membrane at three locations in each eye and after 14 days mice were euthanized, eyes were removed, and choroidal flat mounts were stained with FITC-labeled Griffonia simplicifolia lectin (Vector Laboratories, Burlingame, Calif.) which selectively stains vascular cells (n=12 mice per group). Flat mounts were examined by fluorescence microscopy and the area of each CNV was measured by image analysis with Image-Pro Plus software (Media Cybernetics, Silver Spring, Md.) by an observer masked with respect to experimental groups. In other experiments, wild type 4-6 week-old, female, C₅₇BL/6 mice had rupture of Bruch's membrane in each eye followed by intravitreal injection of 1-30 μg of AGX51 (racemic, E1 or E2) or vehicle in one eye immediately and after 7 days or mice were given twice daily i.p. injections of 500 μg of AGX51 or vehicle for 14 days (n=7-10 mice per group). The area of CNV was measured 14 days after rupture of Bruch's membrane. For Aflibercept and AGX-A treatment experiments, rupture of Bruch's membrane was carried out and then mice were treated with 40 μg of Aflibercept, 10 μg of AGX51E2, 1-5 μg of AGX51, 1-5 μg of AGX-A, DMSO or combinations. The mice were 4-6 week-old female C₅₇BL/6. After 14 days, the mice were euthanized and CNV was measured as described above.

In the ROP experiment 26 C₅₇BL/6 pups, 36 Id^{-/-} and 22 Id3^{-/-} pups were place in 75% O₂ at P7. On P12 the mice were returned to room air and at P17 they were euthanized and retinal neovascularization was measured as described above. In the AGX51 ROP experiment, C₅₇BL/6 pups were place in 75% O₂ at P7. On P12 the mice were returned to room air and injected in the eye with 10 μg AGX51 or DMSO in the FE (N=15 mice/group). On P17 they were euthanized and retinal neovascularization was measured as described above.

Cell Lines and Bacterial Strains

Cell lines. HCT116 (male), 4T1 (female) and 293T (female) cell lines were purchased from ATCC (Manassas, Va., USA) and grown in RPMI (HCT116) or DME (4T1 and 293T) media supplemented with 10% FBS (fetal bovine serum), 1% penicillin-streptomycin and 1% L-Glutamine. HUVECs were purchased from Corning (sex not specified) (Oneonta, N.Y., USA) and grown in EGM-2 media (Lonza, Walkersville, Md., USA). Cells were cultured at 37° C.

Bacterial strains. Id1 and Id3 were purified from Rosetta 2 (DE3) Competent Cell (Sigma Millipore).

### Example 13. Method Details

Id protein purification. pGEV-PSP-mId1 and mId3 expression constructs were transformed into Rosetta 2(DE3) Competent Cells (Sigma Millipore) for protein expression. To produce recombinant GST-tagged protein, a 50 mL LB/Ampicillin (100 μg/m)+Chloramphenicol (25 μg/mL) culture was grown overnight at 37° C. Early the next day, the overnight culture was diluted 1:100 into 6 L LB/Ampicillin+Chloramphenicol and cultured at 37° C. for about four hours until OD₆₀₀=0.7. Cultures were induced with 1 mM IPTG and incubated at 16° C. for 16-18 hours. Cells were harvested by centrifugation at 4000 rpm for 15 minutes at 4° C. and the pellet was resuspended in lysis buffer (50 mM Tris pH 8.0; 400 mM NaCl; 0.5 mM TCEP, Protease Inhibitor Cocktail) (25 mL/L of culture). To lyse the cells, Triton X-100 and lysozyme were added to 0.1% and 10 μg/mL respectively, incubated for 30 minutes on ice and sonicated. Lysates were spun at 17,000 rpm for 30 minutes at 4° C. and supernatants were filtered through a 0.45 μM filter before incubation with glutathione sepharose for two hours at 4° C. The sepharose with bound protein was run through a polypropylene column and washed twice with 50 mL wash buffer (50 mM Tris pH 8.0; 400 mM NaCl) before protein was eluted with 10 mL 50 mM glutathione elution buffer, pH 8.0. Protein was subjected to buffer exchange with PD10 columns (Sigma) into 12 mL storage buffer (50 mM Tris HCl pH 8.0, 400 mM NaCl, 10 mM EDTA, 1 mM DTT, 10% Glycerol).

GST-tag was cleaved with PreScission Protease (0.1 μL of 0.3 μg/μL PreScission Protease cleaves 10 μg of GST-mId1 when incubated at 4° C. for four hours) and incubated with Glutathione Sepharose to separate out the cleaved GST-tag. The cleaved mId1 protein was further cleaned by incubation with GST antibodies (Abcam #ab9085-200 μL anti-GST rabbit polyclonal; ThermoFisher #MA4-004 anti-GST mouse monoclonal) before Coomassie gel analysis. Cleaved protein was concentrated to desired strength using Amicon 3000MCO Ultra-4 centrifugal columns (UFC800308).

Crystallization. Crystals of mouse Id1 (51-104) were grown by the hanging drop vapor diffusion method at 4° C. Aliquots (1.5 μL) of the protein at 2.8 mg/mL concentration in 20 mM Tris buffer (pH 8.0), 0.25 M NaCl and 5 mM DTT were mixed with 1.5 μL of reservoir buffer containing 0.1 M sodium citrate (pH 6.5), 0.2 M magnesium acetate, 10% PEG8000. Crystals were harvested, cryoprotected by stepwise transfer to a solution containing 0.1 M sodium citrate (pH 6.5), 0.2 M magnesium acetate, 11% PEG8000, 30% ethylene glycol and flash-frozen in liquid nitrogen. Crystals of mouse Id1 (58-104) were grown by the sitting drop vapor diffusion method at 4° C. Aliquots of protein at 2 mg/mL (2 μL) in 20 mM Tris buffer (pH 8.0), 0.25 M NaCl and 9% ethanol were mixed with 2 μL of reservoir buffer containing 0.1 M MES (pH 6.5), 0.2 M sodium acetate. Crystals were harvested, cryoprotected by transfer to a solution containing 0.1 M MES (pH 6.5), 0.2 M sodium acetate, 10% PEG8000 and 30% ethylene glycol and then flash-frozen in liquid nitrogen.

Crystals of mouse Id1 (51-104)-human E47 (348-399) complex were grown by the hanging drop vapor diffusion method at 22° C. Aliquots (1 μL) of the protein at 9 mg/mL concentration in 20 mM MES buffer (pH 6.5), 0.3 M NaCl and 5 mM DTT were mixed with 1 μL of reservoir buffer containing 0.1 M potassium phosphate (pH 6.0), 0.25 M NaCl, 22.5% PEG8000. Crystals were harvested, cryoprotected by transfer to a solution containing 0.1 M potassium phosphate (pH 6.0), 0.25 M NaCl, 23% PEG8000, 16% ethylene glycol and flash-frozen in liquid nitrogen.

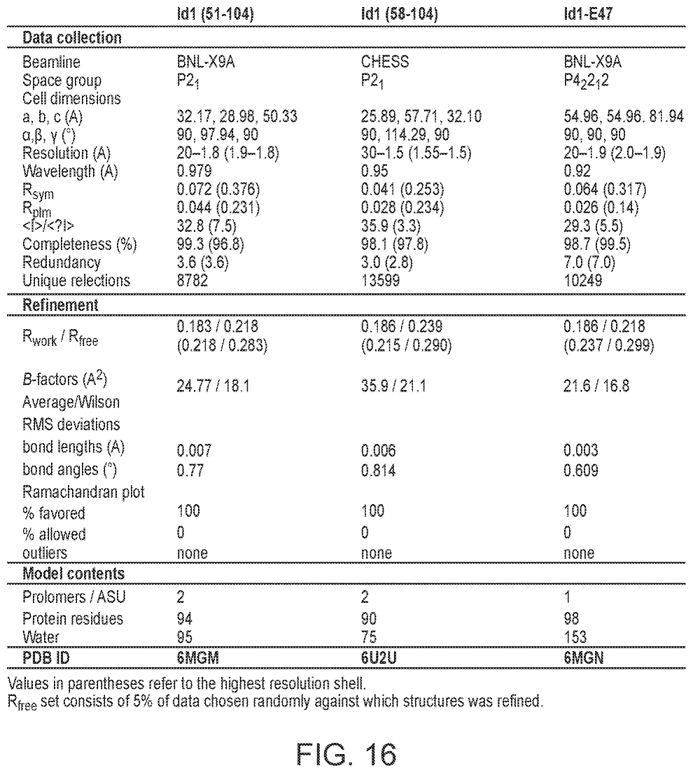

Structure determination. Diffraction data were collected from single crystals at beam line BNL-X9A for Id1 (51-104) and Id1-E47 to 1.8 and 1.9 Å resolution, respectively. For Id1 (58-104) the data were collected at CHESS to 1.5 Å resolution. Indexing and merging of the diffraction data were performed in HKL2000 (Otwinowski and Minor, 1997). The structure of Id1 (51-104) was solved by molecular replacement using PDB entry 1MDY as a search model. The search model was truncated to match the length of the construct used for crystallization. The structures of Id1 (58-104) and Id1-E47 complex were solved by molecular replacement using the refined structure of Id1 (51-104) as the search model. Molecular replacement, model building and refinement were accomplished in Phenix. Diffraction data collection and refinement statistics are summarized in FIG. 16.

In silico screening. Initial docking studies were performed on the Id1-E47 X-ray structure (Deposition ID: D_1000223931 PDB ID: (6MGN)). Compiled lists of commercially available compounds (libraries available from ChemBridge, ChemDiv, Maybridge, and Salor) were screened using a beta release of Autodock 4.0 (The Scripps Research Institute. Molecular Graphics Laboratory. La Jolla, Calif. 92037) using standard settings. For the docking, a cleft adjacent to the loop region of Id1 present in the Id1-E47 heterodimer was targeted. Docking studies were performed on a Sun Microsystems (Menlo Park, Calif. 94025) workstation running Linux. The Monte Carlo simulation for the Id1-small-molecule complex was run for 1×10⁶ steps and 100 conformations were collected and analyzed. The complex conformation with the best score and lowest total energy was selected for further analysis. 3000 compounds that provided promising docking scores, >6.0, were further computationally filtered by computed physical properties: ClogP<5 (the 1-octanol-water partition coefficient), tPSA>80 (topological polar surface area), MW<600 and chemical and biochemical stability. The resulting computational hits, 364 compounds, were purchased from the vendor and screened for their ability to interfere with Id1-E47 homodimerization.

In silico modeling. All ligand preparation and docking calculations used the Schrödinger Suite version 2016-1 using default settings unless otherwise noted. Small-molecules were prepared for docking from sketched 2D structures using LigPrep. 3D structures were generated using the OPLS3 forcefield, and ionization states were determined using Epik at pH 7.0+/−2.0. Id1 monomer, residues 58-104, was prepared from crystallographic coordinates using Protein Preparation Wizard. Protein protonation states were assigned for pH 7.0 using PROPKA. The protein was minimized using the OPLS3 force field. A binding pocket within the Id1 monomer, independent of the in silico screening stage, was identified using SiteMap with default settings, and used to generate a receptor grid for use in docking with Glide where hydroxyl hydrogens (Tyr66, Ser67, Thr75, and Ser83) were allowed to rotate. Docking was performed with Glide using extra precision, flexible ligand sampling, and ring conformation and nitrogen inversion sampling. Torsion sampling was biased for amides to penalize nonplanar conformations.

The BRET probe derived from AGX51 (AGX51 tracer) was prepared in 3 steps from advanced intermediate amine 4 by reaction with commercial N-Boc-aminoPEG2-NHS ester, removal of Boc protecting group, and reaction with Bodipy-558/568-NHS ester.

Circular dichroism. Far UV Circular dichroism (CD) measurements were done on a Jasco J-1500 spectropolarimeter at room temperature using a 0.1 cm cell and a 0.1 mg/mL protein solution in the absence of presence of AGX51, AGX51E1, AGX51E2 or AGX-A. Due to the presence of DMSO in the samples, the lowest wavelength to be scanned is limited.

NanoBRET™ target engagement assay. A N-terminal NanoLuc® luciferase ID1 fusion protein was synthesized by Genewiz (South Plainfield, N.J., USA) into the pUC₅₇ backbone and then cloned into the pcDNA3.1 plasmid with EcoRI-HF and XbaI (both enzymes from New England Biolabs, Ipswich, Mass., USA). The assay was performed essentially as described in the NanoBRET™ TE Intracellular BET BRD Assay kit manual (Promega Corporation, Madison, Wis., USA). Briefly, cells were transfected and after 24 hours were plated onto flat bottom, non-binding surface, white, polystyrene 96-well plates (Corning Incorporated, Kennebunk, Me., USA). The AGX51 tracer was then added (0-4 M), followed by digitonin (Sigma, St. Louis, Mo., USA) to permeabilize the cells (50 g/mL). The NanoBRET™ Nano-Glo® Substrate (Promega) was then added and readings taken using a GloMax Discover System instrument (Promega). For the competition assays, cells were treated with 2 μM of AGX51 tracer and 0-60 μM of AGX-A or AGX51.

Covalent binding of AGX51 derivative to Id1. An analog of AGX51 (AGX51-XL2) was used which contains a benzophenone photoreactive moiety. 1 μg of purified Id1 (a.a.59-104) and 19.7 ng of AGX51-XL2 (dissolved in DMSO) were combined, in the dark, and then exposed to UV light for 20 minutes, a negative control without UV exposure was also included. The samples were then run in the dark on a 15% denaturing gel, silver stained according to the manufacturers protocol (SilverQuest Staining Kit, Invitrogen, Grand Island, N.Y., USA) and bands were excised for mass spectrometry as described below. The above experiment was repeated with the addition of another sample that also had a 10-fold excess of AGX51, relative to AGX51-XL2, to assess the ability of AGX51 to compete with AGX51-XL2.

In-gel digestion for mass spectrometry. In-gel digestion was performed using the method by Shevchenko et al. (Nat Protoc 1(6):2856-60 (2006)). Briefly, gel bands were excised, washed with 1:1 (Acetonitrile: 100 mM ammonium bicarbonate) for 30 minutes, dehydrated with 100% acetonitrile for 10 minutes until gel slices shrunk and excess acetonitrile was removed and slices were dried in a speed-vac for 10 minutes without heat. Gel slices were reduced with 5 mM DTT for 30 minutes at 56° C. in a thermostated mixer with gentle mixing, removed, allowed to cool to room temperature then alkylated with 11 mM IAA for 30 minutes in the dark. Gel slices were washed with 100 mM ammonium bicarbonate and 100% acetonitrile for 10 minutes each. Excess acetonitrile was removed and the slices dried in a speed-vac for 10 minutes without heating. Gel slices were then rehydrated in a solution of 25 ng/μL trypsin in 50 mM ammonium bicarbonate on ice for 30 minutes. Digestions were performed overnight at 37° C. in a thermostated heater with gentle mixing. Digested peptides were collected and further extracted from gel slices in extraction buffer (1:2 vol/vol) 5% formic acid/50% acetonitrile) at high speed mixing. Extractions were combined and dried down in a vacuum centrifuge. Peptides were desalted with C₁₈ resin-packed stage-tips, lyophilized to dryness, then reconstituted in 3% acetonitrile/0.1% formic acid for LC-MS/MS analysis.

LC-MS/MS analysis. LC-MS/MS was performed using a Waters NanoAcquity LC system (with a 100-μm inner diameter×10 cm length C₁₈ column (1.7 μm BEH130; Waters) configured with a 180 μm×2 cm trap column coupled to a Thermo Q-Exactive Plus orbitrap mass spectrometer. Trapping was performed at 15 μL/min 0.1% formic acid (Buffer A) for 1 minute. The LC gradient was 0.5% to 50% B (100% acetonitrile; 0.1% formic acid) over 90 minutes at 300 nL/min. MS data were collected in data dependent acquisition (DDA) mode utilizing a top ten precursor ion selection for HCD fragmentation. Full MS scans were performed with the following parameters: Resolution: 70,000; AGC target: 1e6; Maximum IT: 50 ms; Scan Range: 400 to 1600 m/z. DDA parameters were as follows: Resolution: 17,500; AGC target 5e4; Maximum IT: 50 ms; Isolation window: 1.5 m/z; NCE: 27; Minimum AGC target: 2e3; Intensity Threshold: 4e4; Dynamic Exclusion: 15 s; Charge exclusion: unassigned, 1, 6-8, >8.

Cross/inked peptide identification analysis. MS raw files were processed using Byonic version 2.5 (Protein Metrics, San Carlos, USA) by searching against the mouse ID1 custom database. Search criteria include 10 ppm mass tolerance for MS spectra, 40 ppm mass tolerance for MS/MS spectra, a maximum of two allowed missed cleavages, fixed carbamidomethyl-cysteine modifications, variable methionine oxidation, deamidation on glutamine and asparagine, N-terminal protein acetylation, and the monoisotopic mass of the AGX51-XL2 cross linked product (419.1885 Da). Pep 2D significance threshold of 0.005 or lower were considered significant. Cross-linked peptides were further inspected by visual analysis.

Electrophoretic mobility shift assays. To test the activity of compounds identified in the in silico screen, full-length E47 was purified from bacteria and mixed with a P³² labeled E-box sequence derived from the muscle creatine kinase (MCK) enhancer, BSA, DTT, poly dI-dC, salmon sperm, and HeLa nuclear extract, in the presence or absence of purified full-length Id1. Increasing concentrations of the various test compounds dissolved in DMSO or DMSO alone were added to the reaction mixes for 30 minutes, resolved on a 5% non-denaturing polyacrylamide gel, and autoradiographed. Electrophoretic mobility shift assays (EMSAs) were carried out on whole cell lysates from AGX51-treated cells and the EMSA was performed as described previously (Tournay and Benezra, Mol Cell Biol, 16: 2418-30 (1996)).

Immunoblotting. For immunoblotting, cells were collected by trypsinization, washed with PBS and lysed in homogenization buffer (0.3 M sucrose, 10 mM Tris (pH 8.0), 400 mM sodium chloride, 3 mM magnesium chloride, 0.5% NP40/IGEPAL, 100 μg/mL Aprotinin+Protease inhibitor cocktail (Roche #11 836 153 001). Proteins were separated by SDS-PAGE, transferred to a membrane (LI-COR), probed with primary antibodies overnight at 4° C., and probed with secondary antibodies (LI-COR) for 1-2 hours at room temperature. Proteins were visualized using the LI-COR Odyssey Infrared Imaging detection system. The following primary antibodies were used Id1, Id2, Id3, Id4 (195-14, 9-2-8, 17-3, 82-12, respectively, all from Biocheck), Cyclin D1 (2978, Cell Signaling), Actin (A2066, Sigma), Tubulin (T4026, Sigma). Western blot quantification was carried out using channel 700 and channel 800 intensity data from Odyssey application software version 3.0.30 (LI-COR), subtracting blank values and normalizing to Tubulin.

Immunoprecipitation. Cells were lysed in NP40 lysis buffer (50 mM Tris-HCl, pH 7.5, 150 mM NaCl, 1 mM EDTA, 1% NP40, 1.5 mM Na₃VO₄, 50 mM sodium fluoride, 10 mM sodium pyrophosphate, 10 mM P-glycerolphosphate and EDTA free protease inhibitor cocktail (Roche)) or RIPA buffer (50 mM Tris-HCl, pH 7.5, 150 mM NaCl, 1 mM EDTA, 1% NP40, 0.5% Sodium Deoxycholate, 0.1% Sodium dodecyl sulfate, 1.5 mM Na₃VO₄, 50 mM sodium fluoride, 10 mM sodium pyrophosphate, 10 mM P-glycerolphosphate and EDTA free protease inhibitor cocktail (Roche)). Lysates were cleared by centrifuge at 15,000 rpm for 15 minutes at 4° C. For immunoprecipitation, cell lysates were incubated with primary antibody (FLAG M2 affinity gel, Sigma, F2426; ID1 (C-20), Santa Cruz, sc-488; E2A (N-649), Santa Cruz, sc-763) and protein G/A beads (Santa Cruz, sc-2003) at 4° C. overnight. Beads were washed with lysis buffer four times and eluted in 2×SDS sample buffer. Protein samples were separated by SDS-PAGE and transferred to a polyvinylidene fluoride (PVDF) membrane. Membranes were blocked in TBS containing 5% non-fat milk and 0.1% Tween20, and probed with primary antibodies. Antibodies and working concentrations are: ID1 1:500 (C-20, sc-488) and E2A 1:1000 (N-649, sc-763), obtained from Santa Cruz Biotechnology; HA 1:1000 (C29F4, #3724), obtained from Cell Signaling Technology; (3-actin 1:8000 (A5441), Vinculin 1:8000 (V9131) and FLAG M2 1:500 (F1804) obtained from Sigma. Secondary antibodies horseradish-peroxidase-conjugated were purchased from Pierce and ECL solution (Amersham) was used for detection.

Ubiquitylation assay. HCT116 cells were transfected with pcDNA3-ID1-Flag and pcDNA3-HA-Ubiquitin using lipofectamine 3000 (ThermoFisher). 36 hours after transfection, cells were treated with 60 μM AGX51 for two hours followed by 20 μM MG132 (EMD Millipore) for an additional six hours. After washing with ice-cold PBS twice, cells were lysed in 100 μL of TBS (50 mM Tris-HCl, pH 8.0, 150 mM NaCl) containing 2% SDS and boiled at 100° C. for 10 minutes. Lysates were diluted with 900 μL of TBS containing 1% NP40 and EDTA free protease inhibitor cocktail (Roche) and were cleared by centrifuge at 15,000 rpm for 15 minutes at 4° C. Immunoprecipitation was performed using 1 mg of cellular lysates with FLAG M2 affinity gel (Sigma, F2426). Ubiquitinylated proteins were analyzed by immunoblot using indicated antibodies.

qRT-PCR. RNA was extracted using the RNeasy kit (Qiagen, Valencia, Calif., USA) and cDNA was generated from 1 μg of RNA using SuperScript IV First-Strand Synthesis System (Invitrogen, Grand Island, N.Y., USA). Quantitative PCR was performed using SYBR Green QuantiTect Primer Assay (Qiagen) according to manufacturer's instructions in a 7900HT Fast-Real Time PCR System Instrument (Applied Biosystems, Grand Island, N.Y., USA). Primer pairs for the individual genes were obtained from the bioinformatically validated QuantiTect Library and are as follows: ID1 (QT00230650), ID3 (QT01673336), and GAPDH (QT01192646). The fold changes in gene expression were calculated using the delta-delta CT method.

Cell viability assays. Cell lines were seeded in a 96 well plate (5000 cells per well). After overnight incubation, cells were treated with AGX51 and incubated for 24 hours then MTT reagent (5 mg/mL) was added per well and the cells were incubated for four hours. Following incubation, media was aspirated and 200 μL DMSO was added per well. Absorbance was then measured at 570 nm using a plate reader (Synergy 2, BioTek). Cell growth profiles were determined by seeding 38,000 cells in a 24-well plate, in triplicate for each time point, and counting the cells on days 1, 3 and 5 after seeding, using trypan blue exclusion of dead cells.

Cell cycle analysis. Cells were treated with AGX51 or DMSO, collected by trypsinization, washed with 1×PBS, resuspended in 500 μL 1×PBS and then diluted with 6 mL 70% ethanol and stored at −20° C. until analysis. For cell cycle analysis cells were centrifuged 1000 rpm for 5 minutes, washed with 1×PBS and then resuspended in 0.5 mL PI/RNase staining buffer (550825, BD Biosciences), incubated for 15 minutes at room temperature and analyzed by flow cytometry (LSR II).

HUVEC cell branching assays. For branching assays, 350 μL of Matrigel was loaded into each well of a 24-well plate on ice and incubate the plate for 30 minutes at 37° C. to allow the Matrigel to solidify. 80,000 HUVECs in 0.5 mL of EGM-2 medium with the indicated AGX concentration was plated on the solidified Matrigel. At 18-20 hours of incubation when the tube formation was peaked, media from the well was carefully removed and fixed with 10% buffered formalin for 15 minutes. Each well was washed with DPBS. The morphology of capillary like structures was visualized using an inverted microscope and photographed with a digital camera at 10× magnification. To quantify the tube network, Image J with the Angiogenesis Analyzer plugin (public domain Java-based image-processing program, reference: Carpentier G. ImageJ contribution: Angiogenesis Analyzer. ImageJ News. 2012) was installed and the analysis for the number of nodes, junctions, meshes and total branching length was performed as per the instruction. The statistical data analyses were performed using the Wilcoxon test.

HUVEC scratch assay. HUVECs were seeded on 24-well plate coated with 0.1% fibronectin. After 24 hours when cells were grown to confluency, cells were serum starved for 4 hours in Endothelial Basal Medium (EBM, Lonza) and scraped with a sterile P200 pipette tip to generate a cell free zone. Cells were washed with PBS and stimulated with EGM-2 medium with the indicated AGX concentration for 24 hours. The scratched area at 0 and at 24 hours was visualized using an inverted microscope and photographed with a digital camera at 20× magnification.

Quantification and statistical analysis. Statistical details of experiments can be found herein. Three replicates were generally used for each experimental condition for in vitro experiments and 5 mice per group were typically used in each mouse experiment. The sample sizes were determined based on an expected large effect size. With 3 replicated per condition, an effect size as small as 3 can be detected with 80% power at a two-sided significance level of 0.05 using a two-sample t-test. With 5 mice per group, an effect size as small as 2 can be detected with 80% power at a two-sided significance level of 0.05 using a two-sample t-test. Additional experiments may be performed when larger variation in data was observed and data were pooled for analysis. In general, Welch's t-test was used to examine differences between two groups. ANOVA was used to examine differences across multiple experimental groups. Data may be transformed to ensure the underlying normality assumptions were met. Weighted linear regression analysis was used when heteroscedasticity was observed and data points in each group were typically weighted by the reciprocal of the standard deviation of data in each group. For data pooled from multiple experiments, the model included both experiments and experiments by treatment group interaction as covariates to account for potential differences in experiments. Significance of linear contrasts of interest was assessed based on estimates obtained from the weighted least squares. Q-Q plot of the residuals was examined to ensure the underlying model assumptions were met. P-value <0.05 was considered statistically significant.

### Example 14. Genetic Loss of Id1 or Id3 Reduces Ocular Neovascularization

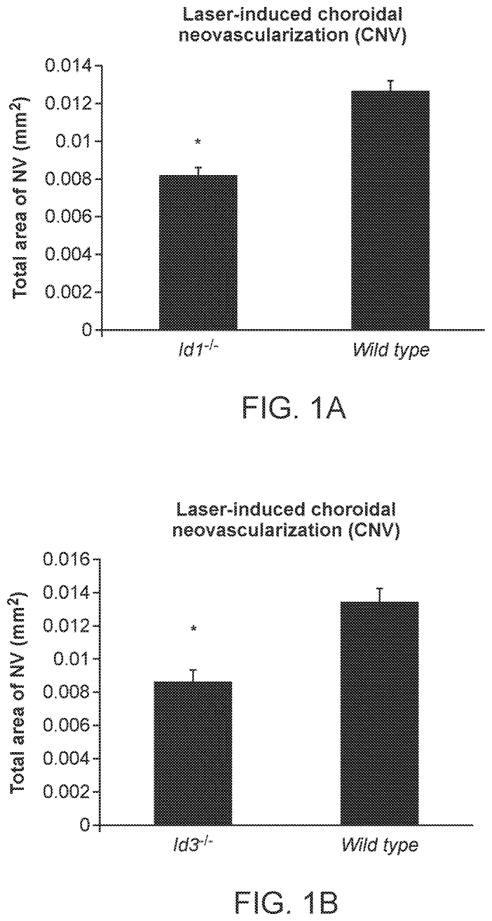

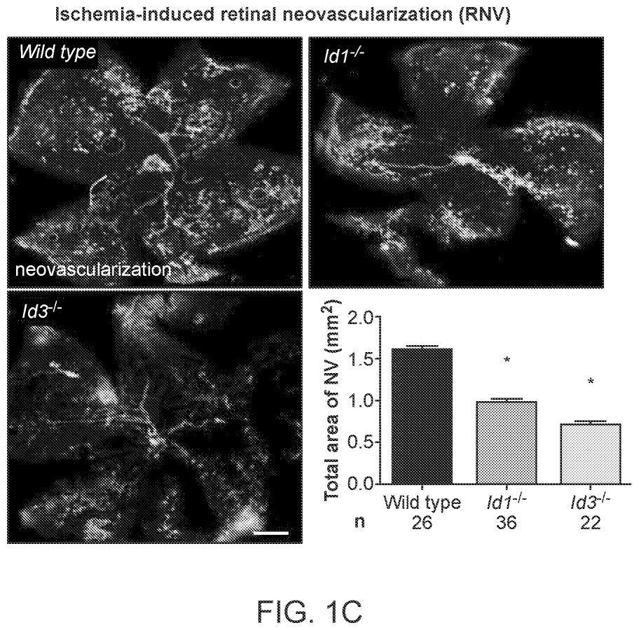

Without being bound by theory, it was hypothesized that Id proteins participate in CNV which occurs in AMD. The mouse model of laser-induced choroidal neovascularization (CNV) used herein has previously provided results predictive of outcomes in clinical trials investigating AMD treatments. Laser-induced rupture of Bruch's membrane was carried out on Id1^{-/-} or Id3^{-/-} mice and littermate control Id1^{+/+} or Id3^{+/+} mice. After 14 days mice were euthanized and choroidal flat mounts were stained with FITC-labeled Griffonia simplicifolia lectin, which selectively stains vascular cells and the area of CNV was measured. As shown in FIGS. 1A and 1B, genetic deletion of Id1 or Id3 significantly suppressed CNV (p<0.03) compared to wild type.

Retinal neovascularization pathology can also be studied in mouse models of retinopathy of prematurity (ROP). In this model, post-natal day (P) 7 mice are placed in a 75% O₂ chamber, which causes retinal capillary depletion. At P12, the mice are returned to room air, which leads to the development of retinal ischemia and proliferative vascular disease in the retinal vasculature. When this model was applied to Id1 or Id3 knockout mice, a significant decrease in retinal neovascularization was observed (p<0.0001) compared to wild type mice (FIG. 1C).

Together these studies demonstrate that antagonizing Id proteins pharmacologically may be a useful approach to treating ocular neovascularization diseases. Accordingly, the compounds of the present technology are helpful for treating ocular neovascularization diseases.

### Example 15. In Silico Screening Identifies AGX51, an Id1 Antagonist

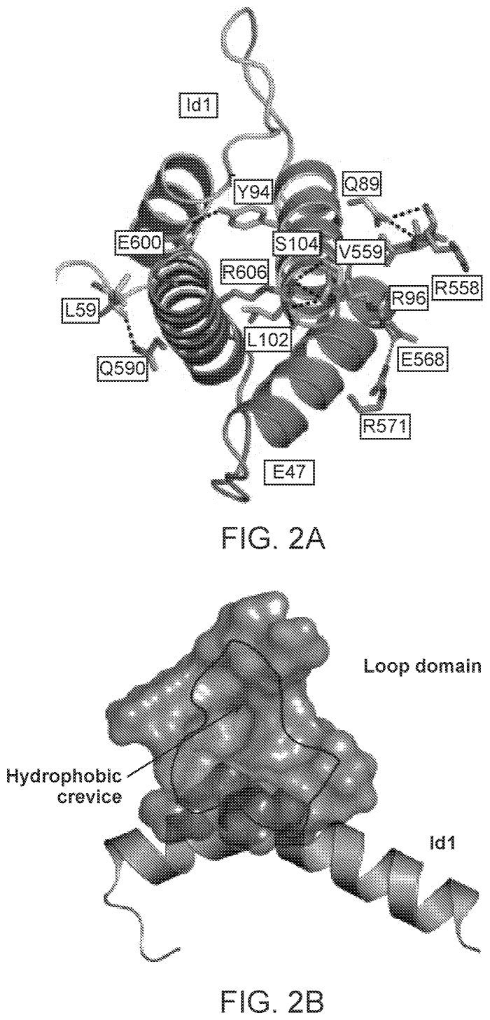

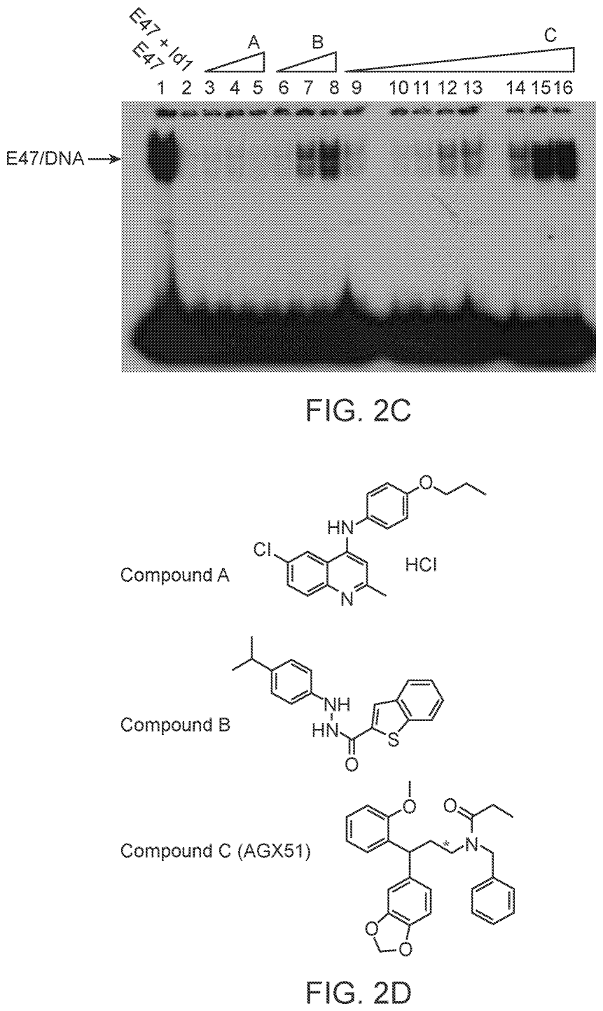

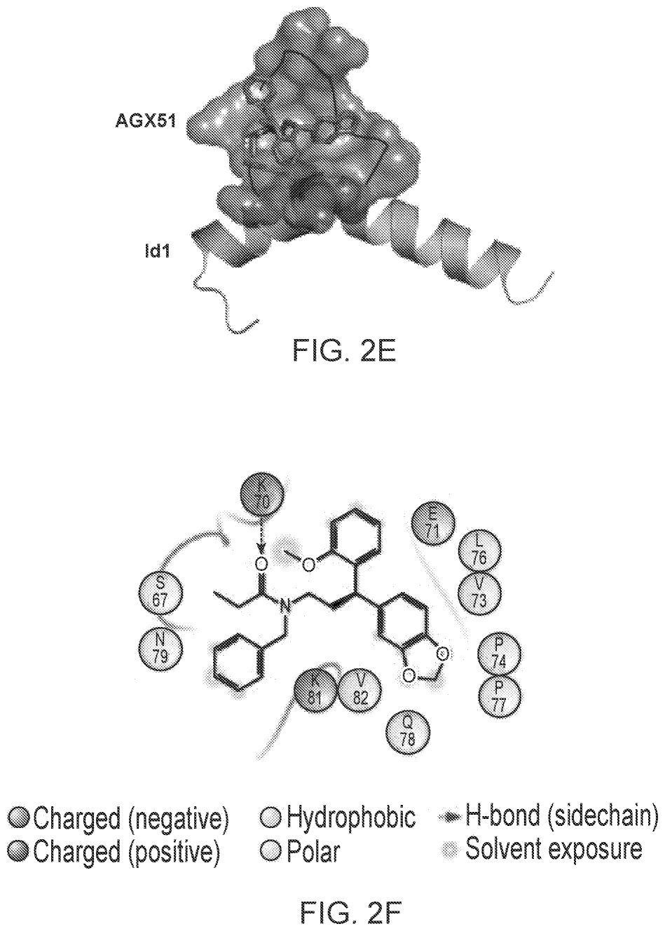

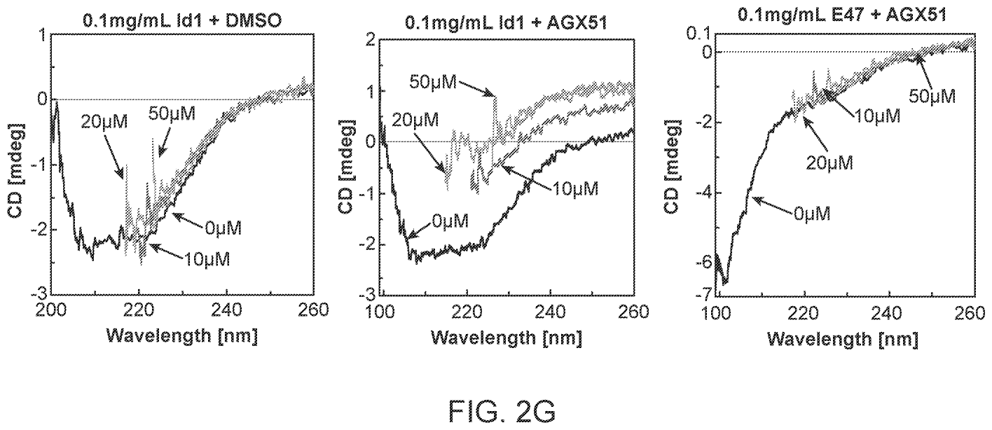

To facilitate the search for small-molecules that may antagonize Id proteins, crystal structures of two fragments of Id1 encompassing the HLH domain, residues (51-104) and (59-104) (identical in mouse and human), and an E47-Id1 complex: Id1 (59-104)-E47 (558-609) were solved. (FIGS. 2A and 16). As shown in FIG. 2A, similar to the other members of HLH superfamily, the structures comprised two α-helices connected by a loop of ten residues in Id1 and seven residues in E47. The crystal structure showed that the HLH domain of Id1 is a homodimer and the α-helices from both monomers formed a four-helix bundle. The interface area is 1045 Å² and is formed by leucine zipper-like regions of hydrophobic interactions and seven hydrogen bonds (FIG. 2A). The Id1-E47 interface is formed by the same region of Id1 as the homodimeric interface. The residues of E47 interacting with Id1 are structurally equivalent to the interface area of Id1, resulting in a four-helix bundle, which is similar to the structure of the Id1 homodimer with a buried area of 1131 Å² (FIG. 2A). In addition to hydrophobic interactions and six hydrogen bonds (Id1-L59: E47-Q590; Id1-Q89: E47-R558; Id1-Q89: E47-V559; Id1-Y94: E47-E600; Id1-L102: E47-R606; Id1-S104: E47-R606), two salt bridges (Id1-R99: E47-E568; E47-E568: E47-R571) are formed (FIG. 2A). The crystal structure further showed that the loop region between the two helices of Id1 is flexible, resulting in different conformations observed in the three structures and high B-factors. Crystal structures of the HLH region of E47 alone (Ahmadpour et al., PLoS One, 7: e32136 (2012)) and in complex with other proteins and DNA were published previously. E1 Omari et al., Cell Rep, 4, 135-47 (2013); Longo et al., Biochemistry, 47, 218-29 (2008). All structures superimposed on the E47 structure reported here with RMSD of 0.4 to 0.8 Å between the Cα atoms. The dimerization interface both in E47 homodimers and heterodimers with T-cell acute lymphocytic leukemia protein 1 and neurogenic differentiation factor 1 was found to be the same as in the Id1-E47 complex. However, these studies showed that the loop region of E47 is more rigid than that of Id1 as the conformations of the loop are similar in all structures of E47.

As shown in FIG. 2B, hydrophobic crevice analysis revealed a cleft adjacent to the loop region of Id1 present in the Id1-E47 heterodimer, a region of Id1 that is highly conserved between members of the family and across species and is critical in maintaining Id activity. Pesce and Benezra, Mol. Cell. Biol., 13, 7874-7880 (1993). An in silico screen of 2,234,000 compounds for crevice binding was performed, yielding 3,000 hits that were pared for drug-like properties. 364 candidates emerged and were tested for their capacity to inhibit the ability of Id1 to antagonize E47 binding to DNA by EMSAs as previously described (Benezra, 1994). A representative EMSA showing three of the compounds tested is shown in FIG. 2C, where compounds B and C showed a dose-dependent increase in E protein binding (lanes 6 to 8 and 9 to 16, respectively). Compound A showed no such recovery. Strong recovery of E protein binding activity relative to E protein alone (compare lanes 1 to 16) suggested that compound C antagonized Id1 activity and had little effect on E47-DNA binding. In summary, the hit rate was 2/364 or 0.55% as only two compounds showed a strong activity in the EMSA assay. This anti-Id1 activity was most likely due to the perturbation of the Id1-E47 interaction but other explanations, such as the binding of an Id1-E47 complex to DNA in the presence of compound, are possible. Previously, EMSAs were carried out in the presence of reticulocyte lysates (Benezra et al., Cell, 61: 49-59 (1990)), and here also HeLa nuclear extract was added to facilitate the observed interactions. Compound C, being more potent than B, was chosen for further analyses as an Id1 antagonist and is referred to as AGX51. The structures of A, B and C from FIG. 2C are shown in FIG. 2D.

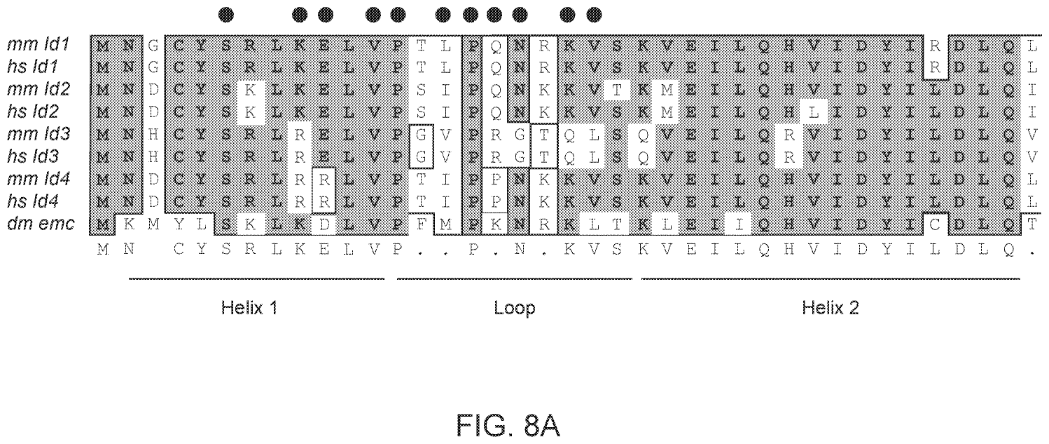

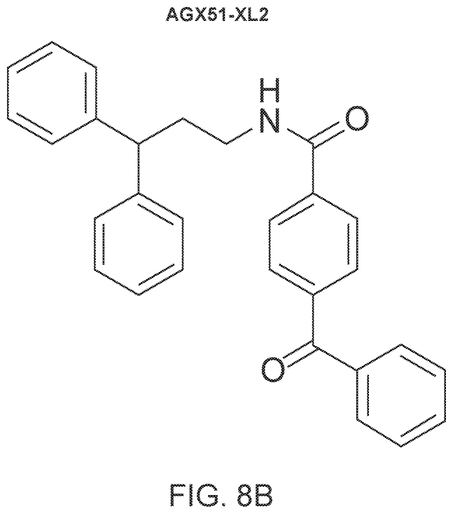

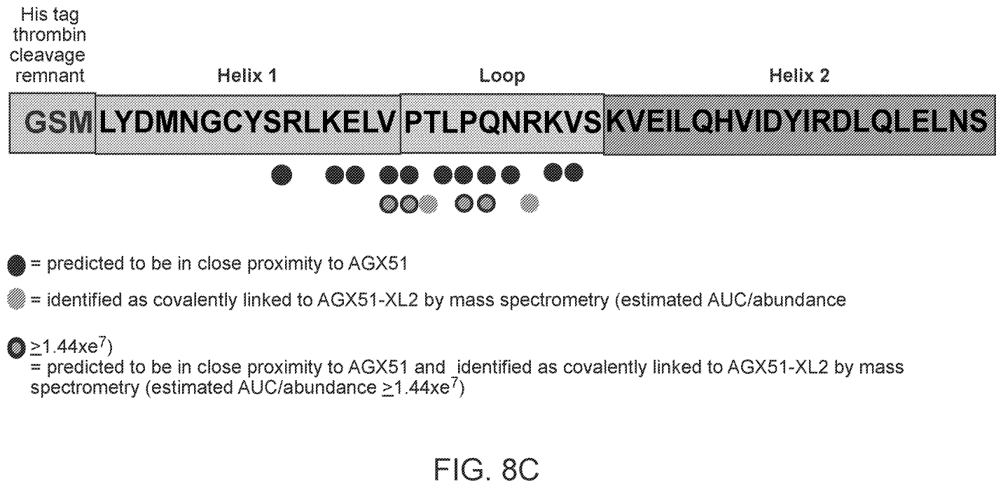

AGX51 possesses a single stereocenter (FIG. 2D). As shown in FIG. 2E, SiteMap (Schrodinger Release 2016-1: Lig Prep, version 3.7, Schrodinger, LLC, New York, N.Y.) was used to predict a binding pocket on Id1, which was the same cleft that was identified in FIG. 2B, while no AGX51-binding pocket on E47 was found. Analysis of the AGX51 binding pose (predicted by a subsequent high resolution docking calculation using Glide XP) showed close proximity of AGX51 to seven residues in the loop domain and four residues in helix 1 of Id1 with Lys70 forming a hydrogen bond with AGX51 (FIG. 2F). Importantly, as shown in FIG. 8, the majority of these residues (7/11) were highly conserved amongst the four Id family members and 9/11 generated a consensus amino acid sequence that is found in the Drosophila melanogaster Id ortholog.

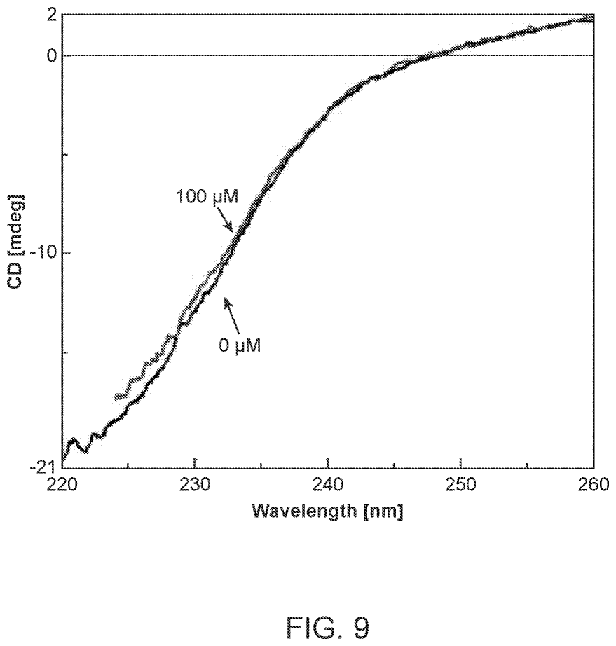

To demonstrate a physical interaction between Id1 and AGX51 we performed circular dichroism (CD) measurements. As shown in FIG. 2G, analysis of the CD spectra showed that Id1 interacted with AGX51, resulting in a significant alteration in the secondary structure of Id1. The CD changes were saturated at 20 μM, implying that the dissociation constant is in this range. Importantly, there was no evidence of an interaction between AGX51 and E47, nor were the changes observed attributable to DMSO or buffer used (FIG. 2G). DMSO has strong absorbance in the wavelengths used in these assays, resulting in spikes in the spectra. Such noise unfortunately was unavoidable since AGX51 is water insoluble and the inclusion of DMSO and the range of wavelengths used in these assays were required. Sufficient quantities of Id1 bearing mutations in the pocket region could not be purified, pointing probably to their inactivity (Pesce and Benezra, Mol. Cell. Biol., 13, 7874-7880 (1993)) and possibly a reflection of the instability of these proteins. The CD assay was also performed with purified Id3, and as shown in FIG. 9, there was a small but reproducible effect of AGX51 on Id3 secondary structure following the trend observed with Id1. While aggregation of AGX51 might affect the CD spectrum of proteins, this is unlikely here since such an effect would not be expected to be specific to the Id proteins over the highly related E47 bHLH protein. Co-crystallization AGX51 and Id1 was also attempted but, these efforts were unsuccessful despite trying over 1000 conditions, including all commonly used sparse matrix screens and soaking the Id1 crystals. Interestingly, when Id1 crystals were exposed to AGX51, but not DMSO alone, the crystals melted. The dissolution of the Id1 crystals after AGX51 exposure is consistent with the CD data, suggesting a conformational change that is incompatible with lattice formation.

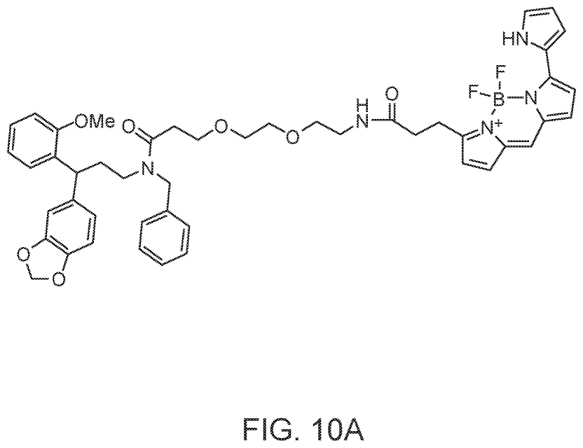

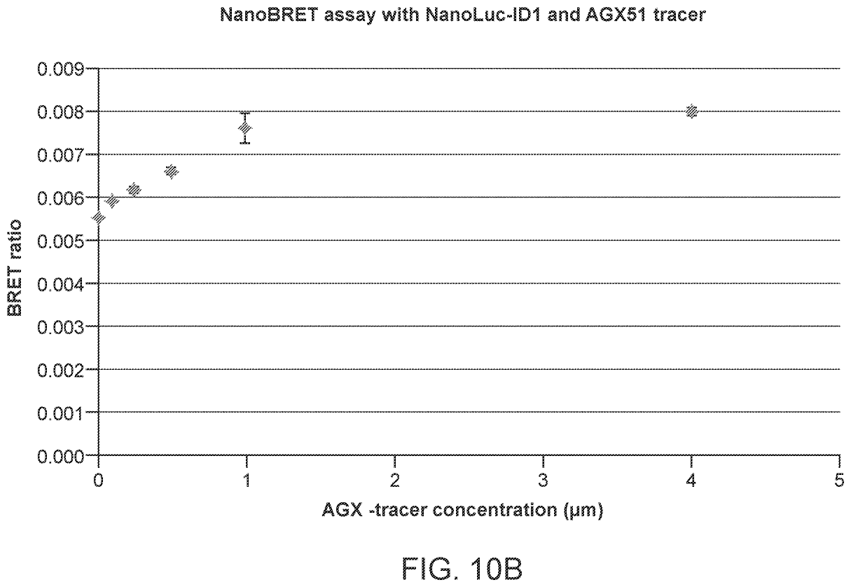

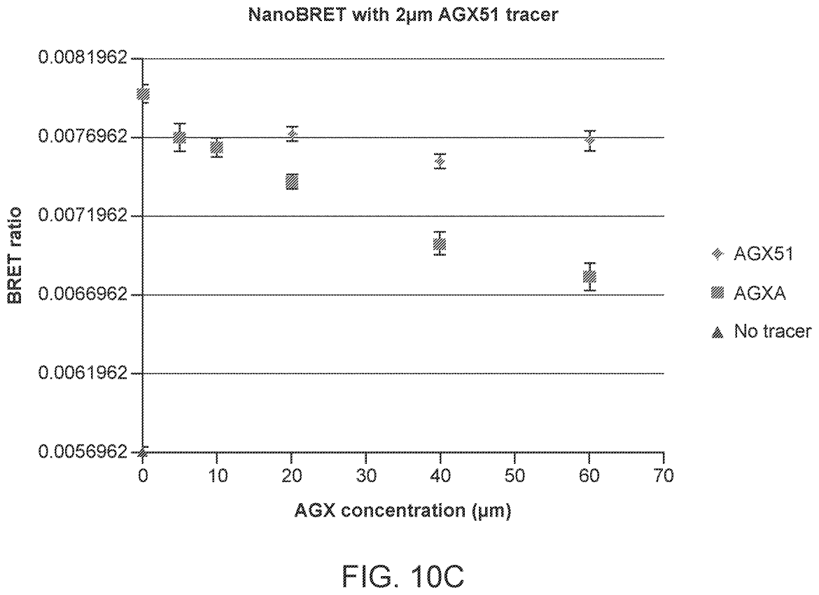

To demonstrate on target engagement in cells, a NanoBRET™ assay was developed for ID1 and AGX51. This assay is based on the NanoBRET™ Target Engagement Intracellular BET BRD Assay (Promega). A construct expressing a NanoLuc® luciferase ID1 fusion protein and an AGX51-fluorescent tracer was generated (FIG. 10A). Upon target engagement bioluminescence resonance energy transfer (BRET) occurs by transfer of the luminescent energy from the NanoLuc® luciferase to the fluorescent tracer that is bound to the target protein portion of the fusion protein. A dose dependent increase in the BRET ratio was observed when permeabilized 293T cells, transfected with the fusion protein, were treated with the AGX51 tracer (0-4 μM) (FIG. 10B), indicating target engagement. Furthermore, as shown in FIG. 10C, the AGX51 tracer could be competed with AGX51 and more efficiently AGX-A, which has greater activity in our biological assays (described herein). The effective compound concentrations identified in these assays vary from those found in biochemical and cell-based assays (see below) as this assay employ the AGX51 tracer, a distinct entity from AGX51 with its own properties and a NanoLuc® luciferase ID1 fusion protein rather than endogenous ID1. Furthermore, the assay was carried out in digitonin-permeabilized cells due to the size of the AGX51 tracer, which may have alter endogenous cellular conditions and hence affect binding properties. These results support ID1 being a direct target of AGX51 in a cellular milieu.

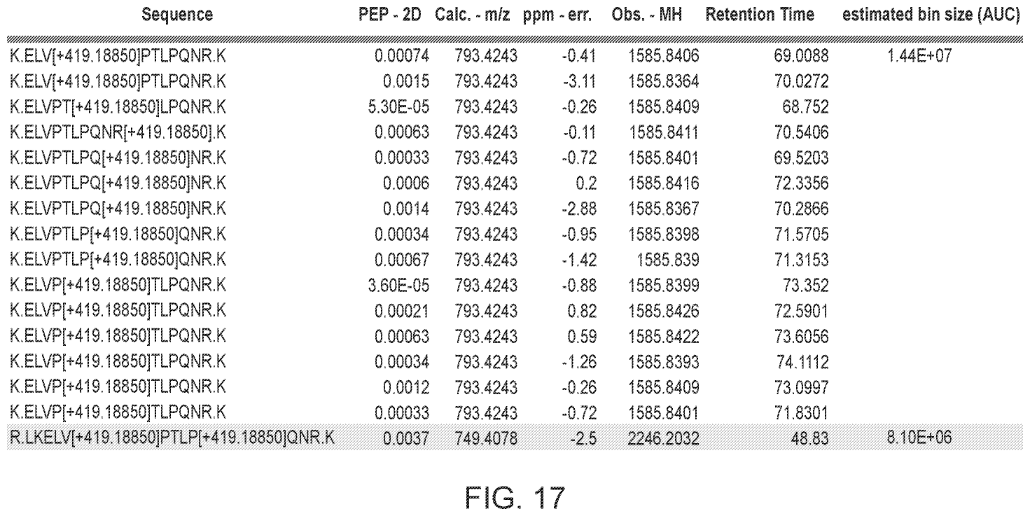

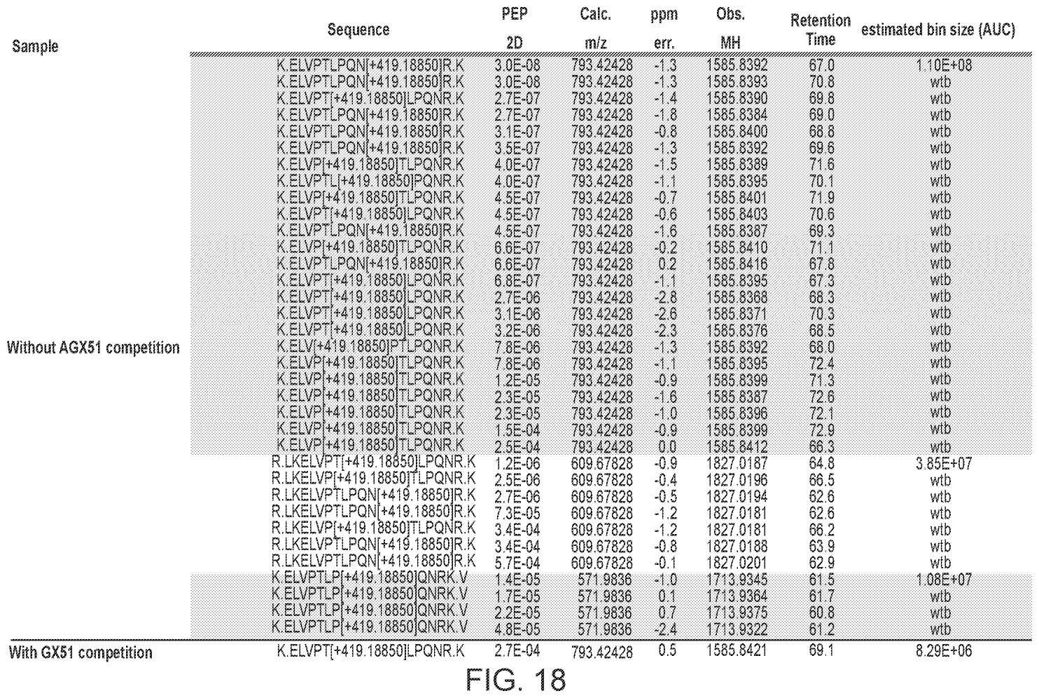

To further validate the physical interaction data, and investigate which Id1 residues interact with AGX51, an AGX51 analog, AGX51-XL2, which incorporates a benzophenone moiety that upon UV irradiation forms a covalent bond with residues in close proximity was used (FIG. 8B). AGX51-XL2 was predicted to bind the same pocket in Id1 as AGX51 (data not shown). AGX51-XL2 and purified Id1 (59-104) were mixed, exposed it to UV light and the samples were analyzed by mass spectrometry. As shown in FIG. 8C, evidence of AGX51-XL2 covalently binding to Id1 at six residues (V73, P74, T75, P77, Q78, and R⁸⁰) was found. These residues overlapped the four helix 1 residues and seven loop residues predicted to be in close proximity to AGX51 (FIGS. 8 and 17). No evidence of covalent binding was observed in the sample not exposed to UV light, nor was there evidence of AGX51-XL2 binding to E47 (data not shown). To support the notion that AGX51 binds the same region as AGX51-XL2, excess amounts of AGX51 was added to compete for the Id1 binding site. Mass spectrometry analysis found a significant decrease in AGX51-XL2 binding to Id1 following the addition of a 10-fold excess of AGX51 (FIG. 18). A cell-permeable, UV-reactive form of AGX51 could not be developed to carry out this analysis in living cells.

These data support a direct interaction between AGX51 and Id1 that is consistent with the in silico screen and modeling described above.

### Example 16. Effects of AGX51 on Id Proteins in Cells

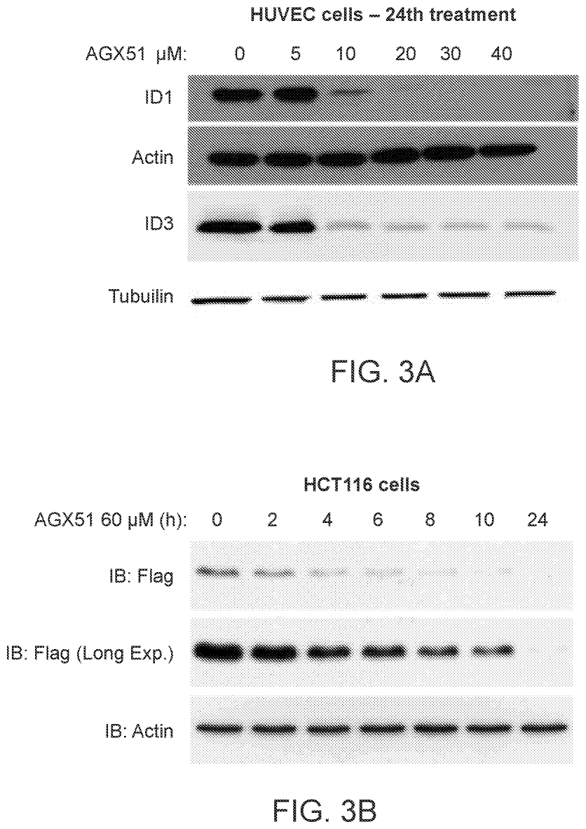

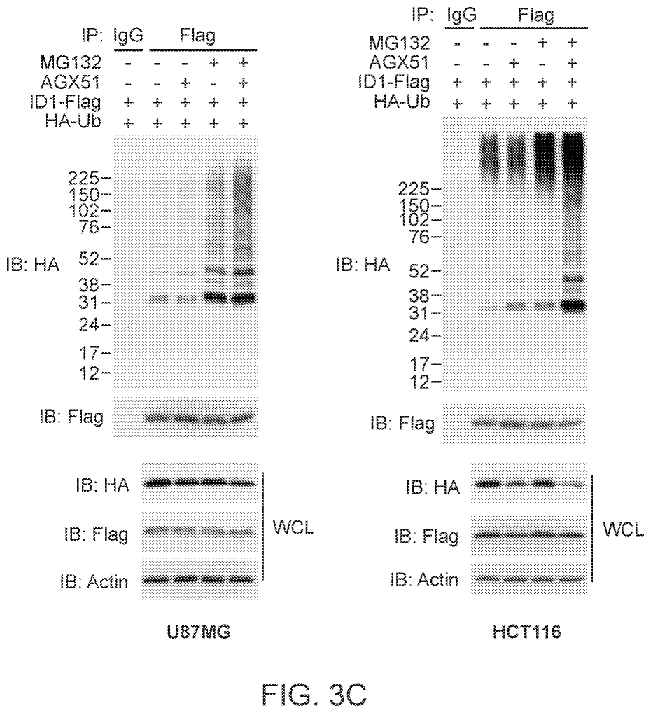

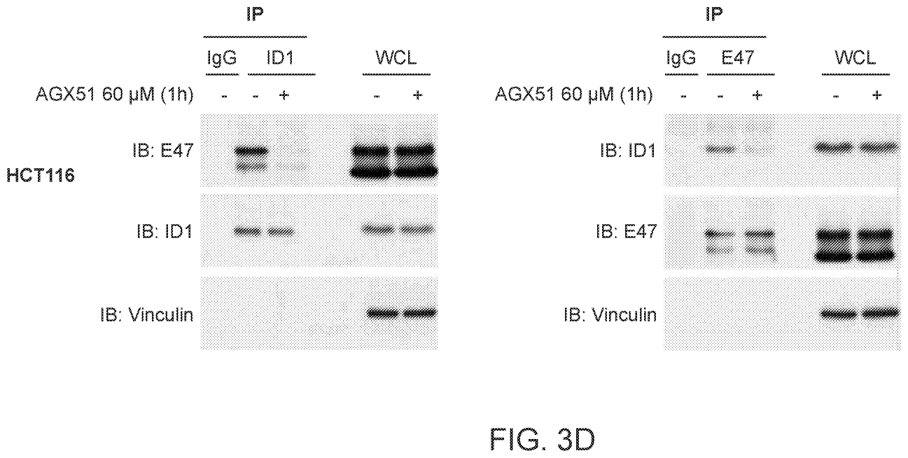

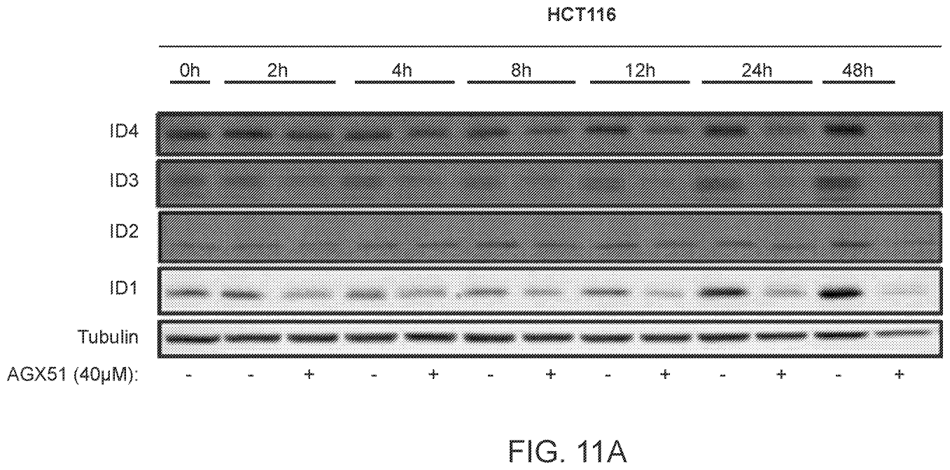

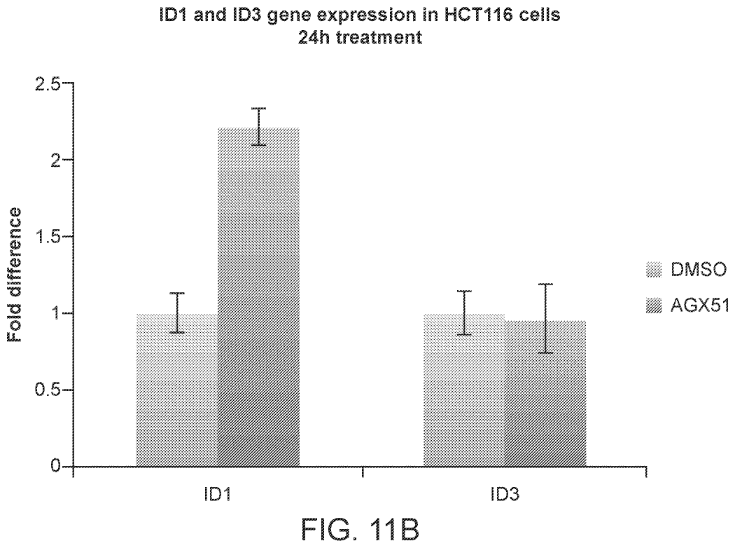

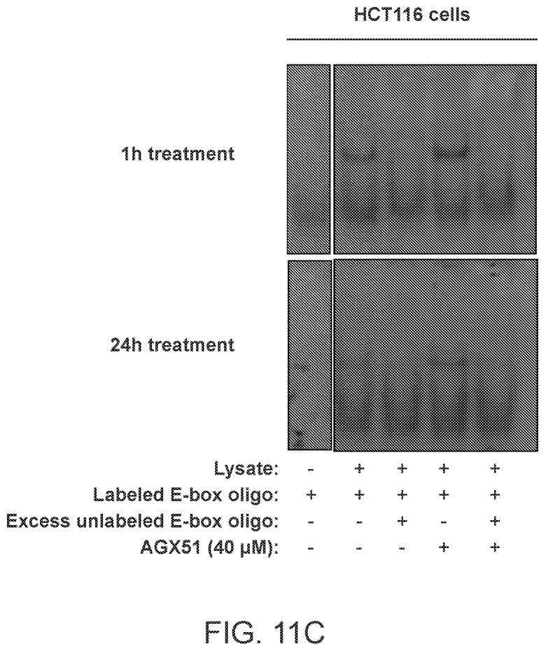

The activity of AGX51 was tested on primary human umbilical vein endothelial cells (HUVECs) and the HCT116 colorectal cancer cell line, two cell types with different ID1-4 expression profiles. Unbound Id proteins are short-lived with half-lives on the order of 10-20 minutes but are significantly stabilized when complexed to E proteins. If Id-E interactions are disrupted by AGX51 in cells in culture as seen in vitro, this would lead to an increase in unbound Id proteins that would then be predicted to be degraded rapidly. HUVECs were treated with increasing concentrations of AGX51 (0-40 PM) for 24 hours and a significant decrease in ID1 protein levels was observed at 10 μM (FIG. 3A). A similar pattern of protein loss was observed for ID3 (FIG. 3A); ID2 and ID4 proteins were undetectable in this cell line (data not shown). The effects of AGX51 on ID3 loss in HUVECs was diminished in the 20-40 μM range compared with ID1, consistent with the weaker perturbation observed in the CD spectra. A similarly reduced effect on Id3 was also observed in 4T1 breast cancer cells (data not shown). In HCT116 cells, AGX51 treatment resulted in reduced levels of ID1, 1D2, ID3 and ID4, suggesting that AGX51 antagonized all four members of the protein family (FIG. 11A). While ID protein levels were reduced by AGX51 treatment, paradoxically, an increase in ID1 mRNA levels was observed (FIG. 11B) perhaps due to activation of the ID1 promoter by liberated E proteins. These results demonstrate that the reduction in ID1 steady state protein levels by AGX51 is strong enough to overcome a significant increase in ID1 mRNA production.

Id proteins are degraded by the ubiquitin 26S proteasome system. Lasorella et al., Nat Rev Cancer, 14: 77-91 (2014). To determine if this degradation pathway mediated the effects of AGX51 on Id protein levels was then explored. HUVECs are difficult to transfect with expression constructs, so HCT116 cells were utilized for this assay. HCT116 cells were transfected with a construct expressing Flag-ID1 and then treated with 60 μM AGX51 for 2-24 hours (FIG. 3B). HCT116 and U87 glioma cells were then co-transfected with FLAG-ID1 and HA-ubiquitin, and treated them either with vehicle or AGX51 for two hours in an attempt to visualize ubiquitylation prior to degradation. The proteasomal inhibitor MG132 was added for an additional six hours prior to immunoprecipitation with an anti-FLAG antibody and immunoblotted using an anti-HA antibody to visualize ubiquitylated ID1. ID1 ubiquitylation was observed in lysates from cells treated with MG132 (FIG. 3C). Treatment with AGX51 further increased ID1 polyubiquitylation in both cell types.

Without being bound by theory, it was hypothesized that loss of Id proteins in response to AGX51 should result in an increase in E protein binding activity assuming little interference of the compound with the E proteins themselves. After treating HCT116 cells with AGX51 for 24 hours, a small increase in E protein binding in cell lysates, relative to controls was observed as expected (FIG. 11C). To determine if loss of Id activity precedes Id protein loss, EMSAs were carried out using cell lysates from HCT116 cells treated with AGX51 for one hour. A similar increase in E protein binding was seen in response to AGX51 at a time when ID1 protein levels are not detectably reduced (FIG. 11C), suggesting that the observed increase in E protein binding was due to disruption of the ID1-E protein heterodimer as opposed to decreased overall ID protein levels. A similar result was observed in 4T1 breast cancer cells treated with AGX51 (data not shown).

To confirm that the increase in E47 binding to DNA in the presence of AGX51 was caused by AGX51-induced dissociation of the endogenous E47-ID1 cellular complex, immunoprecipitation using ID1 or E47 antibodies and western blots for endogenous E47 or ID1, respectively were performed. In both assays, treatment with AGX51 markedly reduced the levels of the co-precipitated proteins prior to any detectable loss of ID1 protein. Thus AGX51 is able to block the ID1-E47 PPI in cells (FIG. 3D).

Together these results suggest that AGX51 treatment disrupted the ID1-E47 complex, leading to proteasomal-mediated degradation of ID1, and the liberation of E proteins to drive transcription, although it is formally possible that the ubiquitinylation event precedes and possibly enhances complete dissociation of the complex followed by Id degradation.

### Example 17. Effects of AGX51 on Cell Growth

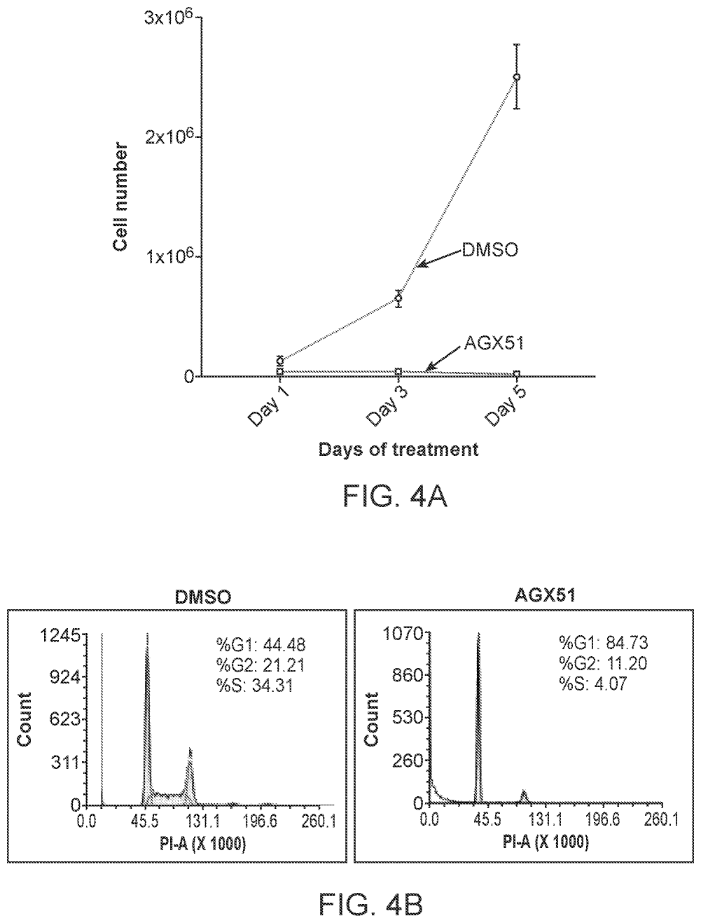

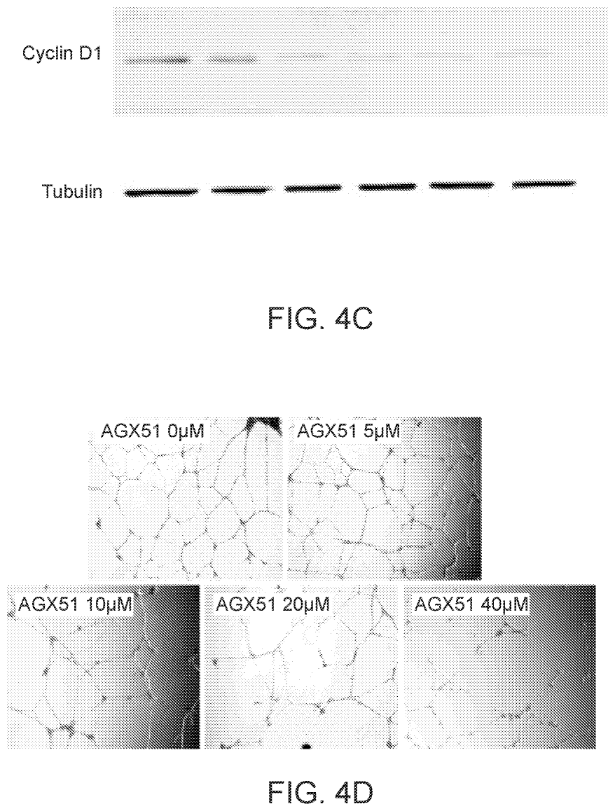

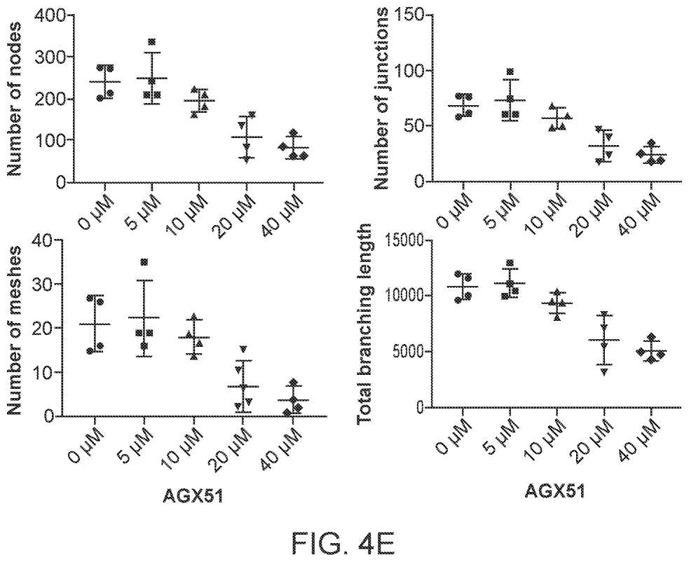

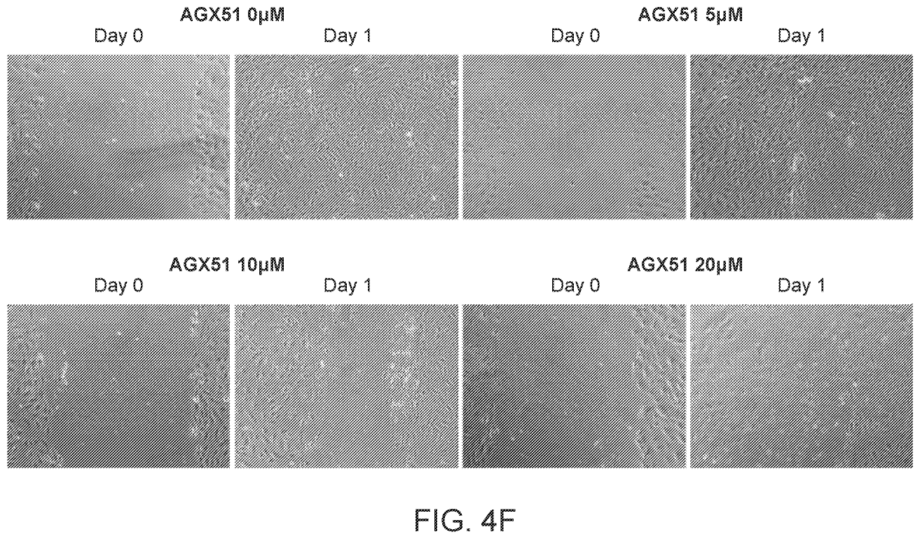

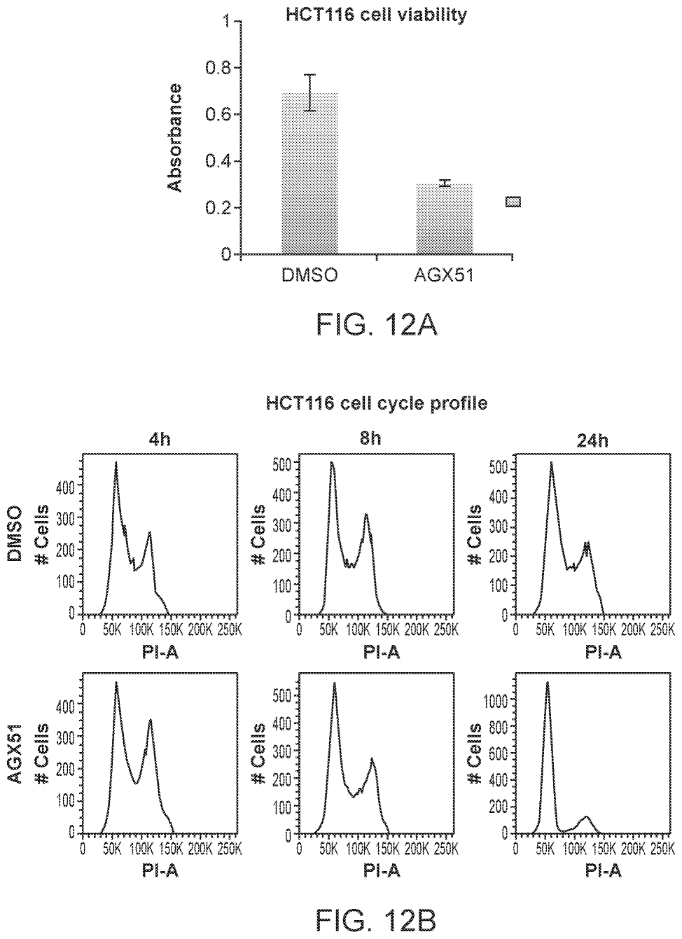

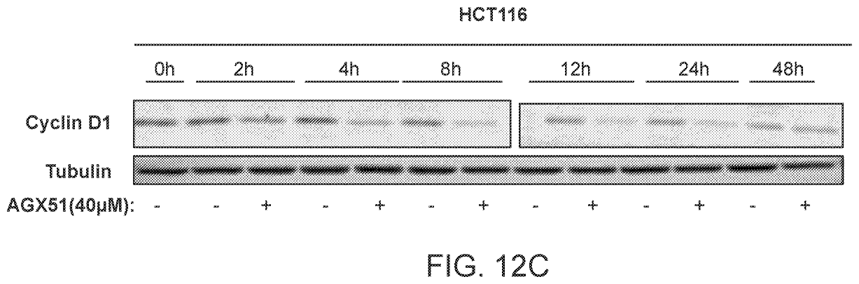

AGX51 treatment resulted in reduced cell viability, G0/G1 growth arrest and a reduction in Cyclin D1 levels in both HUVEC (FIGS. 4A-4C) and HCT116 cells (FIGS. 12A-12C). Other protein changes were also observed by whole proteome SILAC analysis after AGX51 treatment (data not shown). These data are consistent with genetic experiments in which a threshold level of Id protein expression is essential for proliferation and/or viability of essentially all cell types examined in culture but not in most adult tissues in which these proteins are silenced (see toxicity studies below).

To characterize the effects of AGX51 on HUVEC vascular branching the number of nodes, junctions, and meshes as well as branch length were measured following AGX51 treatment. As shown in FIGS. 4D-4E, when HUVECs were cultured on matrigel in the presence of AGX51 for 18-20 hours vascular branching was significantly impaired in a dose-dependent manner across all parameters tested, relative to vehicle control (p<0.05). AGX51 also significantly impaired HUVEC migration after monolayers were scratched and then cultured in AGX51-containing media for 24 hours (FIG. 4F).

Thus, AGX51 treatment impaired normal growth properties of human endothelial cells in culture.

### Example 18. Pharmacokinetics and Toxicity of AGX51 after Intraperitoneal Injection

The determination of the feasibility of administering AGX51 systemically for the treatment of ocular retinopathies was next explored. To determine the half-life of AGX51 in serum, mice were treated by intraperitoneal (i.p.) injection with a single dose of 30 mg/kg or 50 mg/kg AGX51 in 70% DMSO and blood was collected over a 24-hour period. A time-dependent decrease in AGX51 serum levels was observed with a half-life of about three hours. The mean maximum serum concentration of AGX51 achieved following the 30 mg/kg or 50 mg/kg dose was 1.1 and 1.6 μg/mL (2.7 and 4 μM), respectively and was not increased further if mice were treated with a 100 mg/kg dose. Of note, significant Id loss was seen in HUVECs at 10 μM. Higher mean serum concentrations could be achieved in 100% DMSO (˜12 μM at 100 mg/kg) but animals displayed injection site toxicity with DMSO alone. Thus, 70% formulations were used in all future studies.

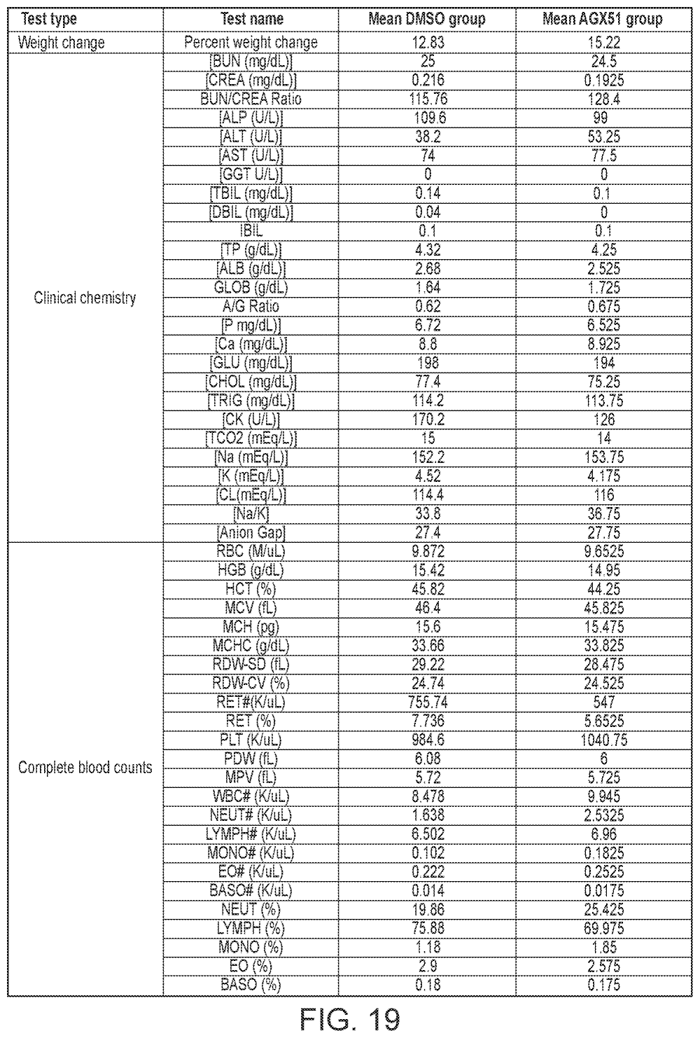

Following a 14-day treatment period where mice were dosed i.p. with either vehicle or AGX51 at 60 mg/kg bid, no mortality or morbidity was observed; in general, all mice looked healthy and displayed normal behavior throughout; no significant weight loss was evident in either group and clinical chemistry parameters and hematology were all within normal limits (FIG. 19). No abnormal findings were detected during gross necropsy, nor following a complete histopathological evaluation of all major organs.

Thus, AGX51 treatment is not toxic. Accordingly, the compounds of the present technology are helpful for treating ocular neovascularization diseases and proliferative diseases, including cancers.

### Example 19. Effect of AGX51 Treatment on Ocular Neovascularization

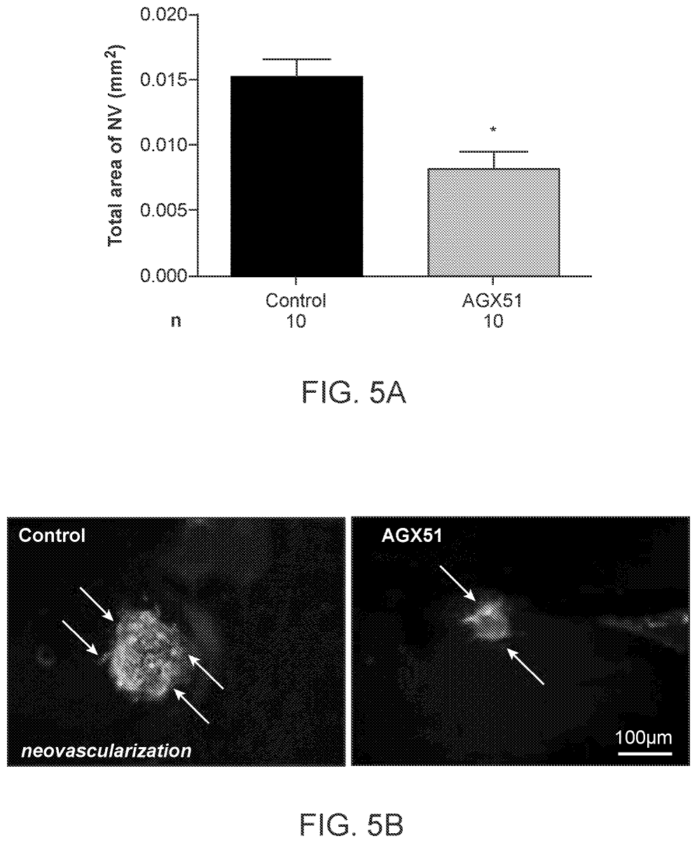

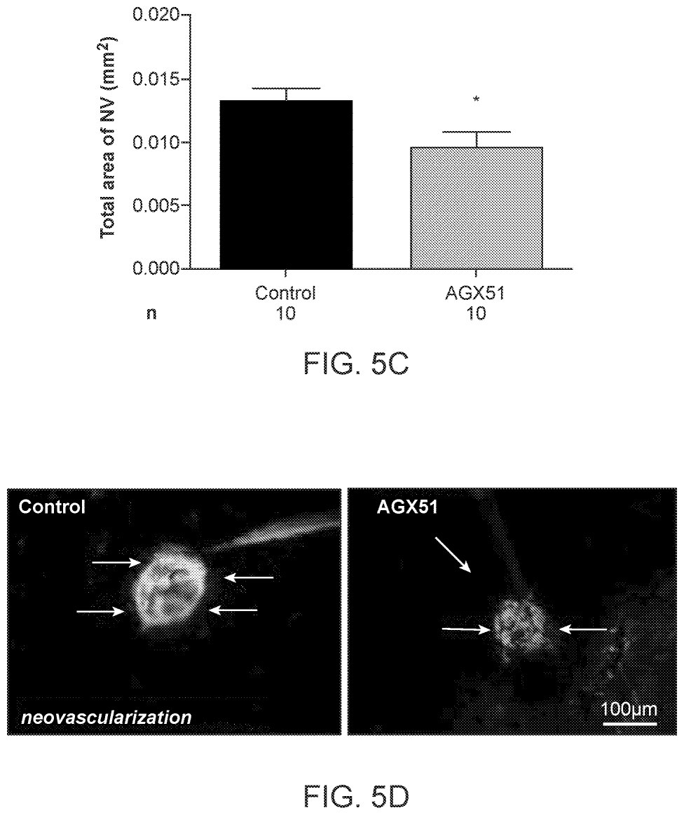

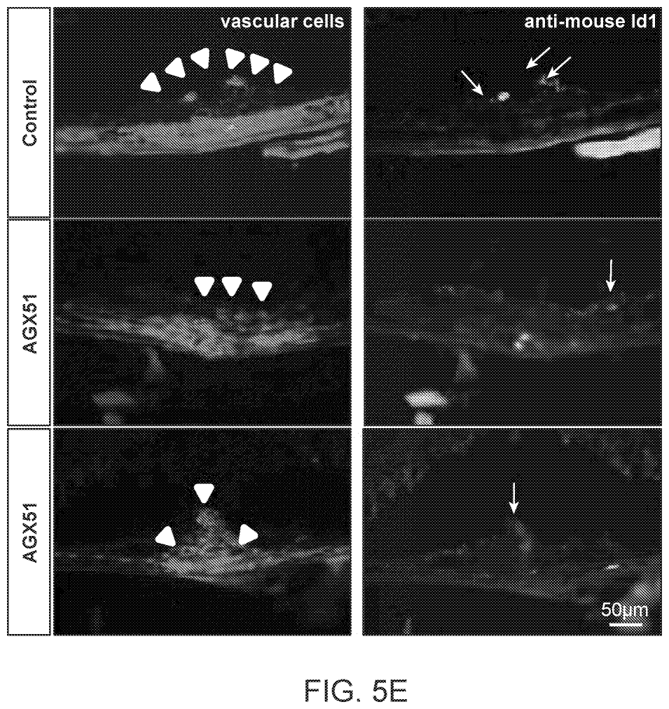

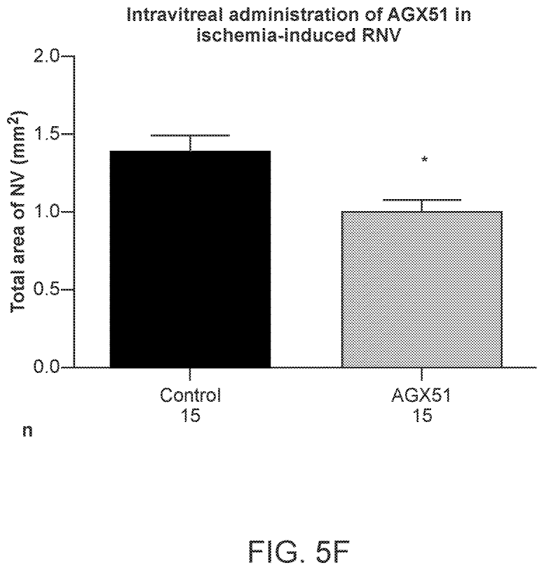

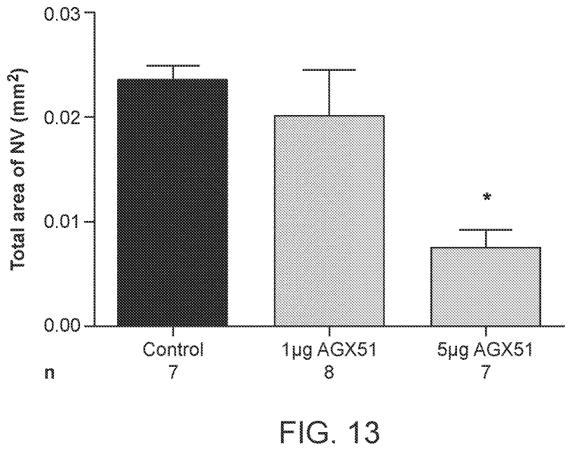

To determine whether AGX51 treatment would phenocopy the effects seen in the genetic models of Id1 and Id3 loss described above, the AMD mouse model discussed herein was again used. Tobe et al., Am. J. Pathol., 153, 1641-1646 (1998). As shown in FIGS. 5A-5B, two intravitreal injections of 10 μg of AGX51 a week apart (2 h and 7 days post laser treatment: analyses carried out on day 14) significantly suppressed CNV relative to vehicle alone (p<0.05). Similar results were seen with a single dose of AGX51 2 h post laser treatment and day 14 analysis (data not shown). 5 μg of AGX51 was also effective at significantly reducing CNV while 1 μg was not (p<0.05) (FIG. 13A). Twice daily i.p. injections of AGX51 (˜30 mg/kg) also significantly reduced CNV relative to vehicle-treated mice (p<0.05) (FIGS. 5C-5D). As shown in FIG. 5E (top row, arrows), Id1 protein was readily detected in control eyes in CNV regions (arrow heads) and co-localized with lectin-stained endothelial cells. Treatment with AGX51 yielded no Id1 positive cells in regions devoid of CNV (see FIG. 5E, middle row) and in rare sections where CNV was observed, there was no Id1 staining (FIG. 5E, bottom row).

The efficacy observed by i.p. administration suggested that AGX51 could reach the eye after systemic injection. The concentration of AGX51 was thus measured in the eyes of mice dosed i.p. with 30 mg/kg AGX51 by mass spectrometry over 24 hours. The maximum concentration of AGX51 after 30 minutes was ˜4 ng/eye, with a 3.7 hour half-life. The amount of AGX51 reaching the eye was well below the amount required by intraocular injection to show efficacy, which could be due to incomplete recovery from the extraction of dissected eyes or that effective dose ranges vary considerably with delivery route.

The effects of AGX51 in the ROP mouse model were also assessed. The mice exposed to hyperoxic and then normoxic conditions were treated with AGX51 at P12 and euthanized them at P17 to measure the extent of neovascularization. As shown in FIG. 5F, intraocular injection of AGX51 significantly reduced retinal neovascularization (p<0.01), consistent with the Id1 and Id3 knockout data. These results are consistent with AGX51 targeting the Id proteins for degradation in regions of CNV, which in turn phenocopies the genetic loss of expression studies.

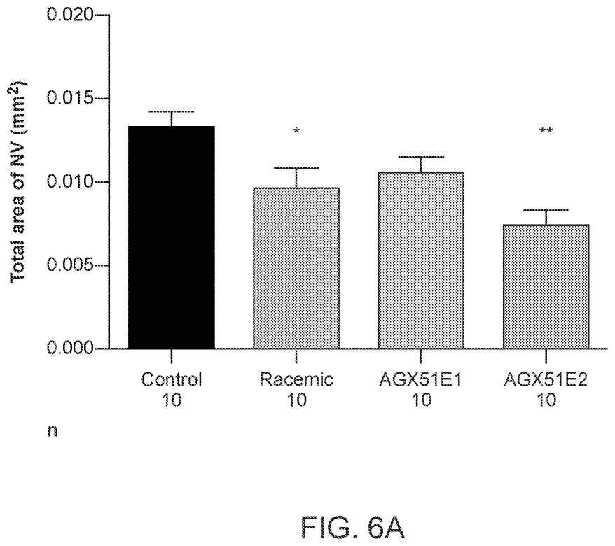

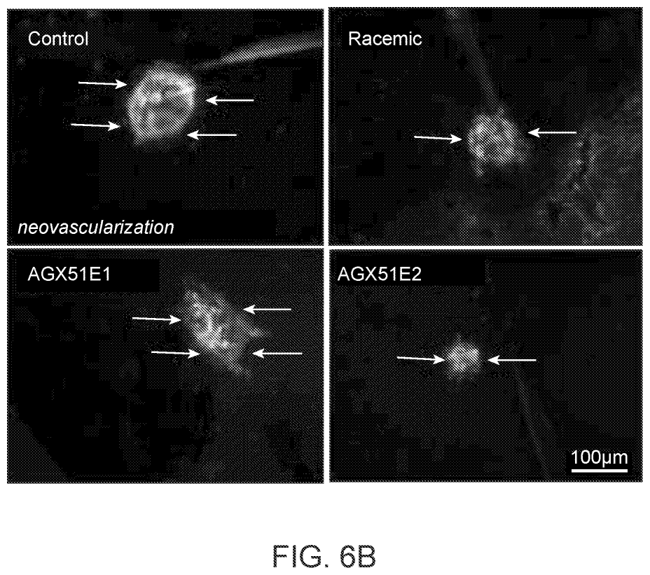

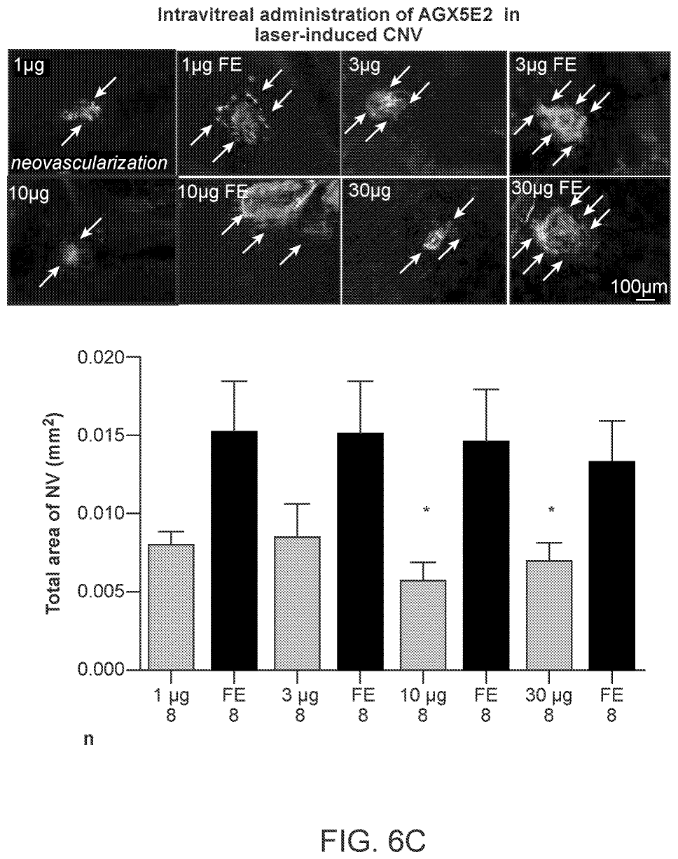

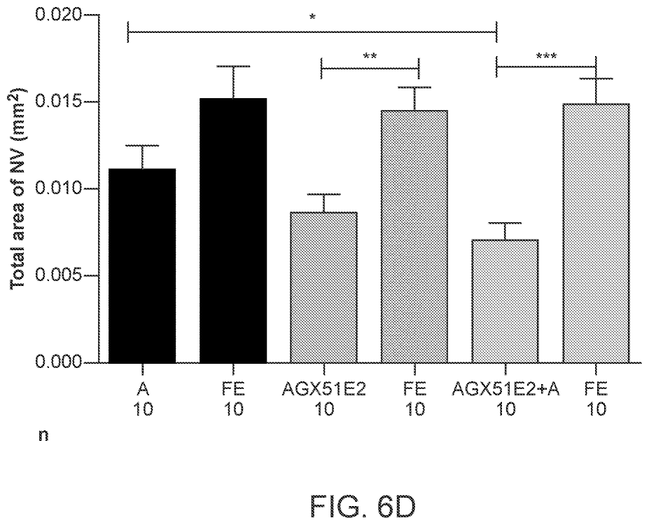

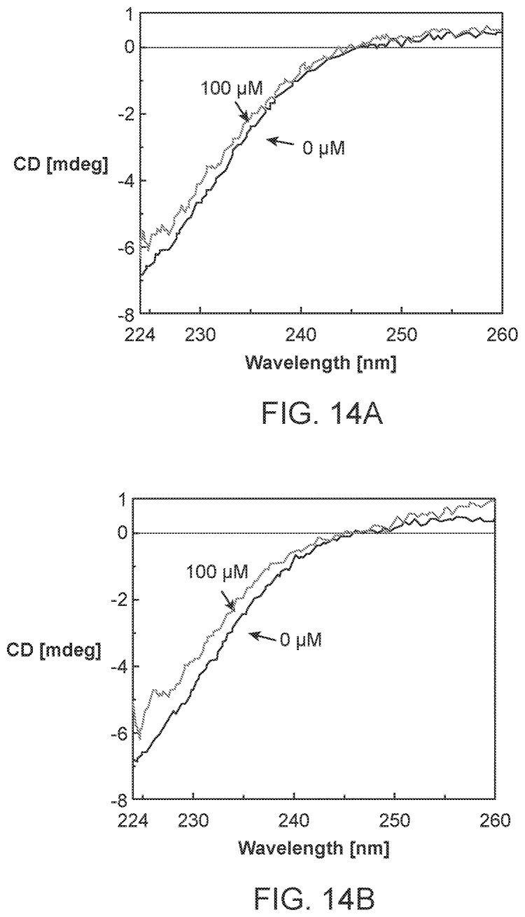

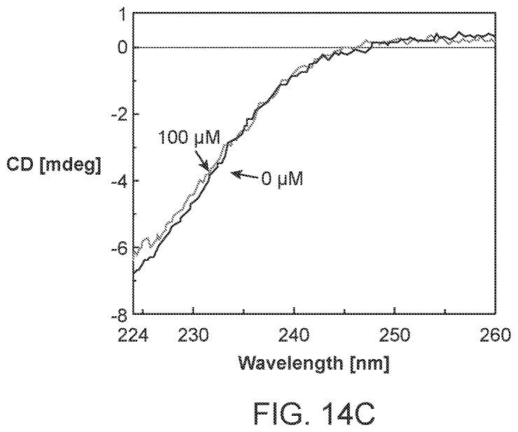

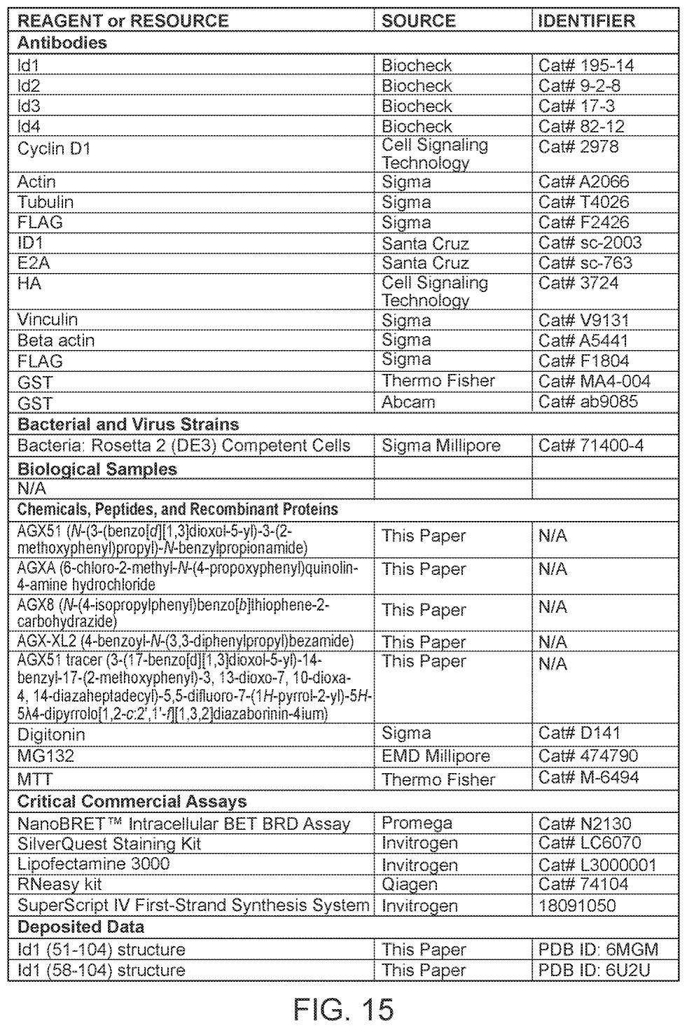

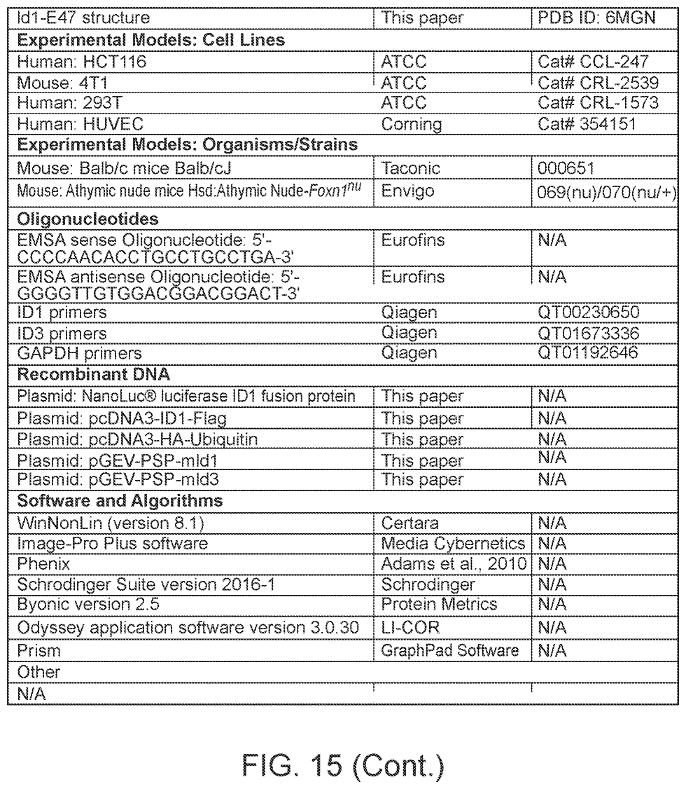

Since AGX51 has one chiral center, determination of the relative activity of the two AGX51 enantiomers (called AGX51E1 and E2) was undertaken. Stereospecific synthesis of the two enantiomers was performed and X-ray crystallization studies identified AGX51E1 and AGX51E2 as the R and S forms of the molecule, respectively. The effects of i.p. injection of the racemic mix, AGX51E1, AGX51E2 and vehicle control were compared in the CNV assay. As shown in FIGS. 6A and 6B, only the racemic mix and AGX51E2 reduced CNV area significantly relative to vehicle control (p<0.05 and p=0.0014, respectively). A dose titration of AGX51E2 in the intravitreal injection assay demonstrated significant efficacy at the 30 and 10 μg dose (p=0.03) relative to fellow eye (FE) but not with the 3 or 1 g dose (FIG. 6C). Interestingly, AGX51E2 showed greater activity than AGX51E1 in the CD assay (FIG. 14), further supporting the idea that it is the more active enantiomer.

Current clinically approved treatment for AMD includes Aflibercept, a VEGF trap, which inhibits the growth of neovessels. To determine the relative efficacy of AGX51E2 and Aflibercept, head-to-head and combination treatment was carried out in the CNV assay. AGX51E2 significantly reduced CNV in this assay relative to the FE (p=0.0014) but Aflibercept, while showing inhibitory activity, failed to reach statistical significance under these conditions. In addition, the AGX51+Aflibercept combination treatment worked better than Aflibercept alone (p<0.05) (FIG. 6D).

Overall, these results suggest that AGX51 targeting of Id proteins in pathologic neovascularization through systemic or intravitreal administration could be a valuable therapeutic approach.

Thus, AGX51 treatment impaired normal growth properties of human endothelial cells in culture.

### Example 20. Characterization of AGX-A

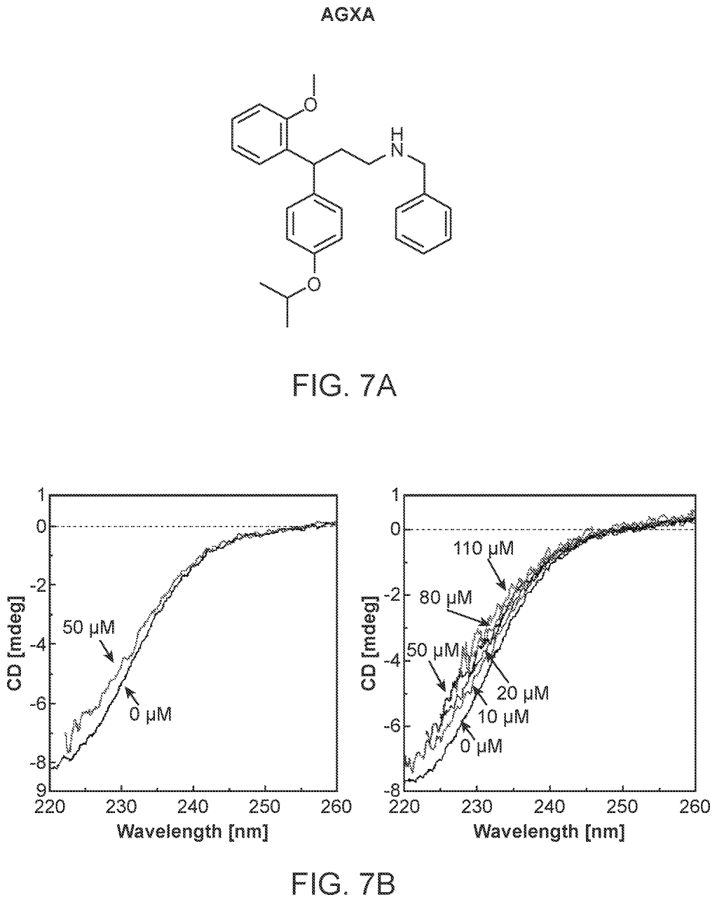

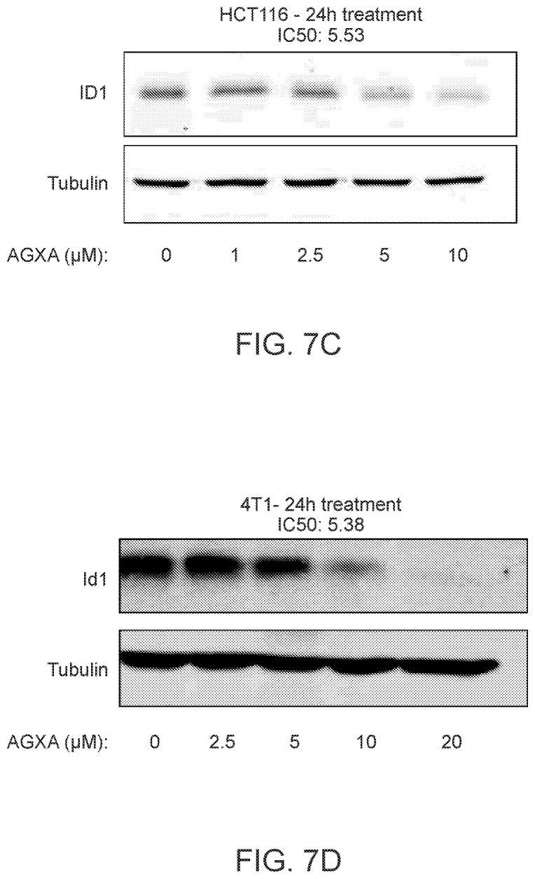

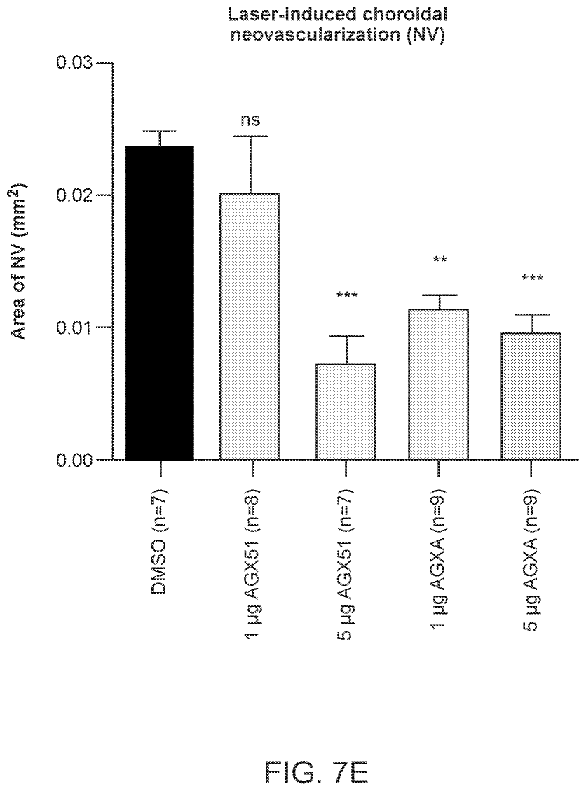

AGX-A has been identified (FIG. 7A) as exhibiting greater activity than AGX51 in CD (FIG. 7B) and NanoBRET assays (FIG. 10C). In cell-based assays, AGX-A showed about a 4-fold reduction in IC₅₀ values and reduced Id protein levels at lower concentrations than AGX51 (FIGS. 7C-7D). Furthermore, AGX-A performed better than AGX51 in the CNV assay at the 1 μg dose (FIG. 7E).

Its shown in the present disclosure that genetic loss of Id proteins reduced neovascularization in two models of ocular vascular disease. AGX51 is a small-molecule antagonist of the Id protein family. This molecule was identified in an in silico screen for compounds that could interact with a hydrophobic pocket within the highly conserved loop region of the Id HLH dimerization motif. CD data demonstrated that AGX51 interacts with Id1 and Id3, and not E47, and alters the Id1 2° structure. The interaction with Id1 occurs in the 20 μM range, consistent with our EMSA data and co-IP data. The concentrations of AGX51 required to see effects against Id proteins are similar to those required for Myc-Max inhibition (˜20-50 μM), the dimer consisting of two bHLH proteins with adjacent leucine zippers that interact to form a four-helix bundle. Importantly, the concentrations of AGX51 used in the in vitro and cell-based assays are in the micromolar range, achieved in the serum of mice after i.p. injection with no associated toxicity, thus suggesting the feasibility of systemic anti-Id therapies. Without being bound by theory, it is hypothesized that the absence of an effect on bone marrow function, and the observation of no overt toxicities in general, are likely due to Id proteins primarily being required by adult stem cells when these cells are called into cycle in response to stress or injury.

The analyses disclosed herein show that AGX51 disrupts the endogenous Id1-E47 PPI in cells, consistent with the proposed model in which AGX51, by binding to a highly conserved and functional loop domain of the Id family, disrupts its ability to associate with E proteins. It is noteworthy that in vitro, cell lysate is required to observe the perturbation of the Id1/E47 interaction suggesting that cellular factors (possibly ubiquitinylation of Id1 upon AGX51 binding) are required to facilitate destabilization of the PPI. Importantly, soon after this PPI is broken in cell culture there is a steady decline in the ID protein levels, which at least for ID1, is due to an increase in ubiquitin-mediated proteolysis. This destabilization of Id proteins is consistent with genetic analyses in which co-expression of E proteins was shown to dramatically increase the stability of Id3. It is noteworthy that none of the other 13 proteins downregulated in response to AGX51 by SILAC analysis (data not shown) are known substrates for the Id1 de-ubiquitinase (USP1) making USP1 an unlikely target of the drug. As AGX51 appears to act as an Id protein antagonist and degrader in cell culture and tissues the levels of Id proteins in tissues or circulating Id-expressing cells could potentially serve as biomarkers of AGX51 activity.

While the loss of Id proteins in response to AGX51 treatment both in cells and in animals clearly indicates that they are drug targets, without being bound by theory it is hypothesized that Id proteins are the critical targets in AGX51-induced phenotypes. If true, one would expect the compound to recapitulate Id loss-of-function mutation effects and this prediction has been borne out in multiple assays: AGX51 inhibits cell proliferation, inducing a G0/G1 arrest; inhibits ocular neovascularization in mouse models of AMD and ROP; and phenocopies the effects of Id1 and Id3 loss in a variety of cancer models including ROS production and metastasis suppression (data not shown). In addition, partial reduction of Id1 and Id3 with shRNAs reduced the IC₅₀ of AGX51 in cells and cell killing is severely attenuated in quiescent cells in which Id proteins are undetectable (data not shown). One cannot yet rigorously rule out the possibility that other unintended targets also contribute to the phenotypes observed.

Results from CD, crosslinking and NanoBRET™, assays as well as crystal lattice perturbation presented here support direct target engagement between 131 and AGX51 both in vitro and in cells. Furthermore, AGX-A worked at lower concentrations than AGX51 to effect secondary changes in Id1 in CD assays and to compete for tracer binding in the NanoBRET assay; correspondingly, AGX-A degraded Id proteins in cells at lower concentrations than AGX51, has a lower IC₅₀ and stronger activity in the CNV assay. Conversely, higher concentrations of AGX8 (compound B in FIG. 2) were required to perturb Id1 in the EMSA assay, degrade Id proteins in cells and showed a higher IC₅₀ in cell viability assays (data not shown). Together these data support that AGX51 and AGX-A directly engage the target ID proteins.

Intravitreal and/or systemic administration of AGX51 suppressed ocular neovascularization in two models of neovascular ocular disease: AMD and ROP. Importantly, efficacy in the ROP model may also be predictive of that in diabetic retinopathy. Furthermore, it is shown herein that AGX51 performed as well as the currently available AMD treatment, Aflibercept, and combination therapy worked better than Aflibercept alone. The ability of systemic delivery of AGX51 to inhibit neovascularization in the retina is consistent with intravitreal delivery and with the possibility that Id-dependent circulating endothelial progenitors, are contributing to the phenotype. While the molecular signals promoting neovascularization are not necessarily identical in all organs, the involvement of Id proteins in ocular neovascularization and tumor angiogenesis, suggests that AGX51 may have therapeutic potential in other diseases complicated by neovascularization.

In conclusion, herein identified is a first-in-class Id protein antagonist and degrader, AGX51, that phenocopies Id genetic loss in pathologic states suggesting that in addition to being a useful biologic tool for studying Id proteins, it can also be developed into a therapeutic agent that may provide clinical benefit in a variety of Id-related human pathologies, including ocular neovascularization diseases and proliferative diseases, including cancers.

### Example 21. Comparative Studies of Anti-Id Compounds AGX51 and AGX-A for Anti-Id Efficacy, Anti-Cancer Efficacy, and for Targeting Resting Stem Cells and Preventing Acquired Resistance Associated with Standard of Care Chemotherapy in Cholangiocarcinoma

This example provides side-by-side comparative studies for AGX51 and AGX-A. These studies illustrate the broad and flexible scope of compounds of the present technology to mediate anti-Id, anti-cancer, and anti-pathogenic vascularization as well as supporting other therapeutic effects in clinical use. The studies here show that compounds of the present technology such as AGX-A are potent anti-Id drugs, performing well as effective, dose-dependent anti-cancer compounds in accepted animal models of cancer drug efficacy in humans. As discussed in further detail below, AGX-A exhibits significantly superior potency compared to AGX51 in several experiments, as well as comparable or significantly superior efficacy at lower doses.

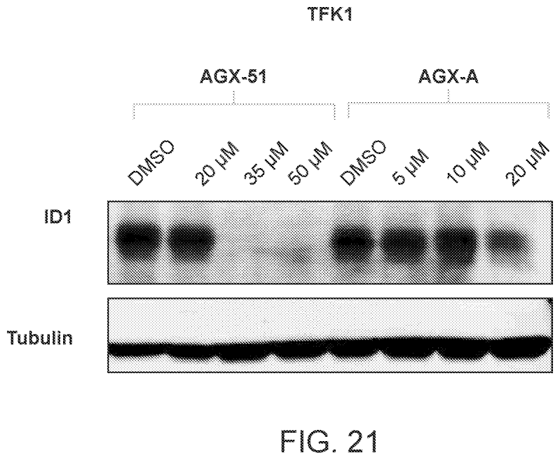

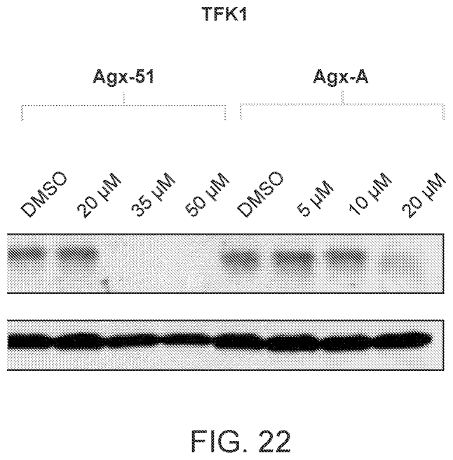

Following the methods described herein (see, e.g., Examples 12-13), AGX-A and AGX51 were compared for their ability to mediate Id knockdown in cell cultures. FIG. 21 illustrates side-by-side comparative effects of AGX51 and AGX-A for knockdown of Id1 in TFK1 cell cultures, and FIG. 22 illustrates side-by-side comparative effects of AGX51 and AGX-A for knockdown of Id3 in TFK1 cell cultures. Notably, while most relevant cell lines in culture don't express Id3 (e.g., SNU1079, SNU1196), TFK1 does express Id3 in culture. In these experiments, AGX-A exhibits superior anti-Id1 and anti-Id3 potency compared to AGX51, including showing comparable or superior Id knockdown activity at lower concentrations as compared to AGX51.

A clinical target of cholangiocarcinoma was selected for further comparative studies for several reasons. Preliminary studies showed that cholangiocarcinoma cells and tumors overexpress Id1 and are also positive for Id3, implicating these targets as amenable to anti-Id1 and anti-Id3 intervention. Significantly, cholangiocarcinoma is a refractory cancer type often exhibiting relapse with acquired resistance following first-line standard of care (SOC) treatment using the potent chemotherapeutic drug gemcitabine. The expanded studies below show that anti-Id therapy per the present technology targets Id1 and Id3 positive “resting” stem cells. These cells representing a pool of cancer progenitor cells relatively resistant to chemotherapy (based on their resting, non-proliferative state, such cells elude first line chemotherapy targeting proliferative cells). As such these stem cells frequently escape first line cancer treatment, whereafter they are capable of rebounding to give rise to new cancer cell populations. Yet additional evidence presented here shows that compounds of the present technology also penetrate in their effects to preclude acquired resistance, either alone or in combination with conventional chemotherapeutic cancer therapies. For example, resting stem cells or cancer cells that mutationally escape first line treatment (e.g., cells that acquire resistance to chemotherapeutic drugs through mutation) cannot further elude or develop resistance to the compounds of the present technology. Without being bound by theory, the functionally critical, evolutionarily constrained/conserved Id binding interface (targeted by the compounds of the present technology) is practically immutable to generate “acquired resistance” because any structural mutation yields a biologically inoperative Id protein, functionally null for the essential purposes such proteins serve in cancer cells.

Yet additional data herein further evidence that the anti-Id compounds of the present technology potently target resting stem cells that escape first line chemotherapy, radiation and other cancer treatments targeting highly proliferative cell populations. Histochemical studies were performed where the results demonstrated that resting, non-proliferative stem cells in two distinct tumor types, cholangiocarcinoma and triple negative breast cancer (TNBC) express Id1 mutually exclusively with Ki67, a marker for cancer cell proliferation. Comparable data showed that non-proliferative stem cells likewise mutually exclusively express Id3 and not Ki67. Further studies showed that a ratio of Id1+/Ki67+ cells dramatically increased in tumors following chemotherapy, meaning the relative population of resting stem cells that are Id1+/Ki67− was greatly enriched by chemotherapy. These data indicate that Id1 are both resting stem cell markers in diverse cancer types, that resting Id+/Ki67− stem cells are resistant to conventional chemotherapy (and other cancer treatments that target proliferation); and that the anti-Id compounds of the present technology eradicate resting Id+ stem cells implicated in cancer recurrence following chemotherapy/remission. For targeting these resting stem cells, anti-Id compounds of the present technology may effectively be employed in coordinate treatment protocols with chemotherapeutics or other conventional cancer treatments, as a second-line therapy following first-line treatment with chemotherapeutics or other conventional treatments, or alone as an effective first-line treatment that uniquely, broadly targets both proliferative cancer cells and resting, Id+ stem cells.

A: Comparative Effects of AGX51 and AGX-A on Cell Growth

In vitro studies examined anti-Id-mediated impacts of AGX51 and AGX-A on diverse cholangiocarcinoma cell lines, listed in Table 1 below.

TABLE 1
Representative Cholangiocarcinoma Cell Lines Studied

| Cell Line | Derivation |
|---|---|
| TFK-1 | Extrahepatic cholangiocarcinoma, primary tumor |
| SNU-1079 | Intrahepatic cholangiocarcinoma, primary tumor |
| SNU-1196 | Extrahepatic cholangiocarcinoma, primary tumor |
| HUCC-T1 | Intrahepatic cholangiocarcinoma, malignant ascites |
| EGI-1 | Extrahepatic cholangiocarcinoma, primary tumor |
| WITT | Extrahepatic cholangiocarcinoma, malignant ascites |

The effects of AGX51 and AGX-A on cholangiocarcinoma cell viability were assessed for the six different cholangiocarcinoma cell lines. The measured IC₅₀ values (in μM) from these studies are provided in Table 2 below, along with the ratio of the AGX51 IC₅₀ to the AGX-A IC₅₀ to aid in comparison.

TABLE 2
IC50 Values for AGX51 and AGX-A in Various Cholangiocarcinoma Cell Lines

| | AGX51 | AGX-A | (AGX51 IC50)/ |
|---|---|---|---|
| Cell Line | (IC50 in μM) | (IC50 in μM) | (AGX-A IC50) |
| TFK-1 | 15.69 | 11.80 | 1.33 |
| SNU-1079 | 23.08 | 5.84 | 3.95 |
| SNU-1196 | 28.76 | 14.79 | 1.94 |
| HUCC-T1 | 14.80 | 4.44 | 3.33 |
| EGI-1 | 15.68 | 4.49 | 3.49 |
| WITT | 33.24 | 5.32 | 6.25 |

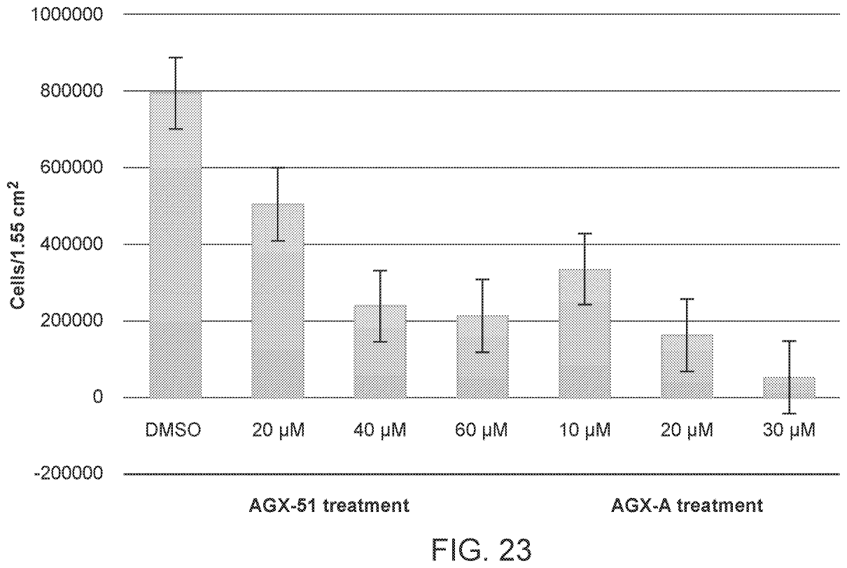

As illustrated in Table 2, both AGX51 and AGX-A exert Id-knockdown related negative impacts on cholangiocarcinoma cell viability, with the anti-cancer properties of AGX-A consistently superior to that of AGX51. In particular, the anti-cancer properties of AGX-A significantly superior to that of AGX51 in SNU-1079, EGI-1, and WITT cells. The dose-dependent effects of AGX51 and AGX-A on TFK1 cell viability was assessed in related studies over a 24 hour period, where exemplary data are illustrated in FIG. 23. These data demonstrated that, compared to AGX51, AGX-A possesses superior anti-cancer potency with comparable or significantly superior inhibition of cell viability at lower concentrations.

B: In Vivo Efficacy of AGX-A, with and without Gemcitabine in a Murine Model of Cholangiocarcinoma

Additional studies in an animal model of cholangiocarcinoma predictive of anti-cholangiocarcinoma drug efficacy in humans evidence the dose-dependent anti-cancer efficacy of AGX-A alone as well as for AGX-A in combination with the SOC chemotherapy drug gemcitabine.

A first round of the study (“Round 1”) was planned as indicated in the Round 1 Study Protocol blow, utilizing gemcitabine in saline and AGX-A in DMSO for administration.

### Round 1 Study Protocol—Animal Model and Experimental Design (AGX-A in DMSO):

Mice: NSG females, 6-8 weeks old
PDX: RomeP_PHCH_X_0008a, serially transplanted sc with matrigel
Treatment Groups (at least 6 animals/group): treatments started when tumors reached 100 mm³ (“Day 0” for each animal)
   Group 1. Saline, i.p. once a week (“QW”) for 3-4 weeks (“×3-4 weeks”)
   Group 2. Gemcitabine 25 mg/kg i.p. QW×3-4 weeks
   Group 3. AGX-A 10 mg/kg i.p. once a day for 5 consecutive days (“QD×5”)×3-4 weeks
   Group 4. Gemcitabine 25 mg/kg i.p. QW+ AGX-A 10 mg/kg i.p. QD×5×3-4 weeks

Tumor volumes, weights, and clinical signs were monitored a minimum of twice per week for each animal in each group throughout the study.

In the Round 1 study, it was observed that animal weight in Groups 1 and 2 was essentially unchanged for the duration of the study. However, for animals receiving AGX-A in DMSO—i.e., Groups 3 and 4—weight loss was observed, indicating that the DMSO was exhibiting some degree of toxicity. Because of this, treatment in Groups 3 and 4 was halted after Day 9 while tumor volumes, weights, and clinical signs continued to be monitored a minimum of twice per week for each subject in each group. Subsequently, the animals in Groups 3 and 4 stopped loosing weight and instead started regaining weight. Meanwhile, treatment in Groups 1 and 2 continued as planned. Due to the alterations in the planned study, Round 1 was concluded at Day 20.

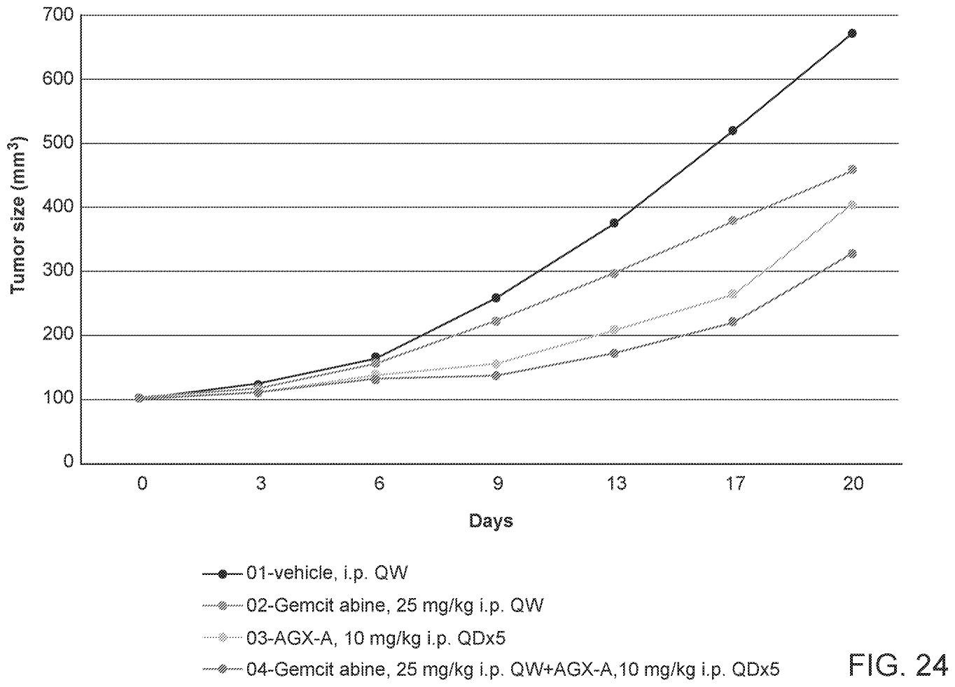

Tumor volume data from the Round 1 study is presented in FIG. 24. Notably, even though treatment of Group 3 was discontinued at 9 days, the data shows AGX-A alone performs better than the SOC drug gemcitabine. Further, the data for Group 4 shows that combination therapy employing AGX-A with gemcitabine (again, where treatment was discontinued after only 9 days) yields even greater anti-cancer effects.

To overcome the issue DMSO posed, investigations were performed to develop a formulation that was compatible with AGX-A as well as well-suited for administration. One investigation explored use of sulfobutylether-β-cyclodextrin (CAPTISOL) with anti-Id compounds of the present technology, however formulations with sulfobutylether-β-cyclodextrin neutralized AGX-A induced cell death in cholangiocarcinoma cells.

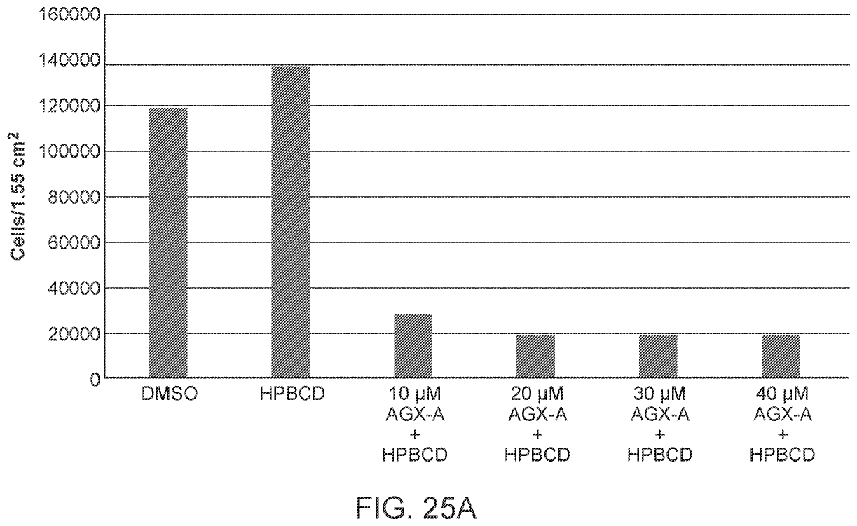

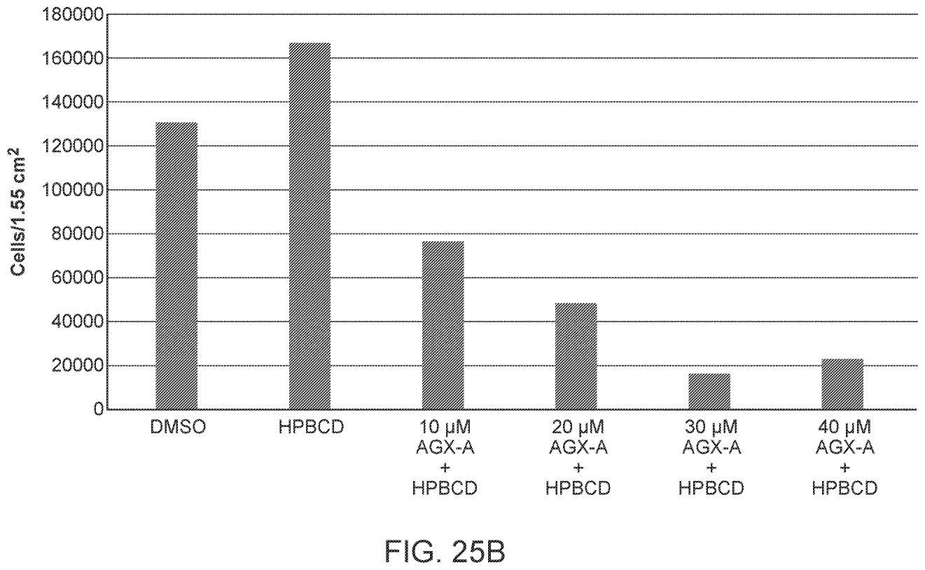

Ultimately, a particularly efficacious formulation employing 2-hydroxypropyl-β-cyclodextrin (“HPBCD”) was discovered. HPBCD was found to effectively solubilize AGX-A in aqueous solution while preserving AGX-A activity against cholangiocarcinoma cells. Representative data is illustrated in FIGS. 25A-25B which provide the results of 24 hour cell viability studies for SNU1079 cells and SNU1196 cells, respectively. These studies and data evidence that HPBCD and comparable solubilizing agents may effectively be employed in pharmaceutical formulations including anti-Id compounds of the present technology.

With this discovery in hand, a “Round 2” study was performed as indicated below, where AGX-A was administered in a saline formulation including 12.5% by weight HPBCD while gemcitabine was administered in saline (as gemcitabine was administered in the Round 1 study).

### Round 2 Study Protocol—Animal Model and Experimental Design (AGX-A+HPBCD):

Mice: NSG females, 6-8 weeks old
PDX: RomeP_PHCH_X_0008a, serially transplanted sc with matrigel
Treatment Groups (at least 6 animals/group): treatments started when tumors reached 100 mm³ (“Day 0” for each animal)
   Group 1. Saline with 12.5% by weight HPBCD (“Vehicle”), i.p. QD×5 each week×1 week
   Group 2. Gemcitabine 15 mg/kg i.p. QW×1 week
   Group 3. AGX-A 10 mg/kg i.p. QD×5×1 week
   Group 4. AGX-A 15 mg/kg i.p. QD×5×1 week
   Group 5. Gemcitabine 15 mg/kg i.p. QW+ AGX-A 10 mg/kg i.p. QD×5 1 week
   Group 6. Gemcitabine 15 mg/kg i.p. QW+ AGX-A 15 mg/kg i.p. QD×5 1 week

During the study, tumor volumes, weights, and clinical signs were monitored a minimum of twice per week for each animal in each group. No weight loss was observed for the animals in any group throughout the duration of this Round 2 study, evidencing HPBCD is well-tolerated.

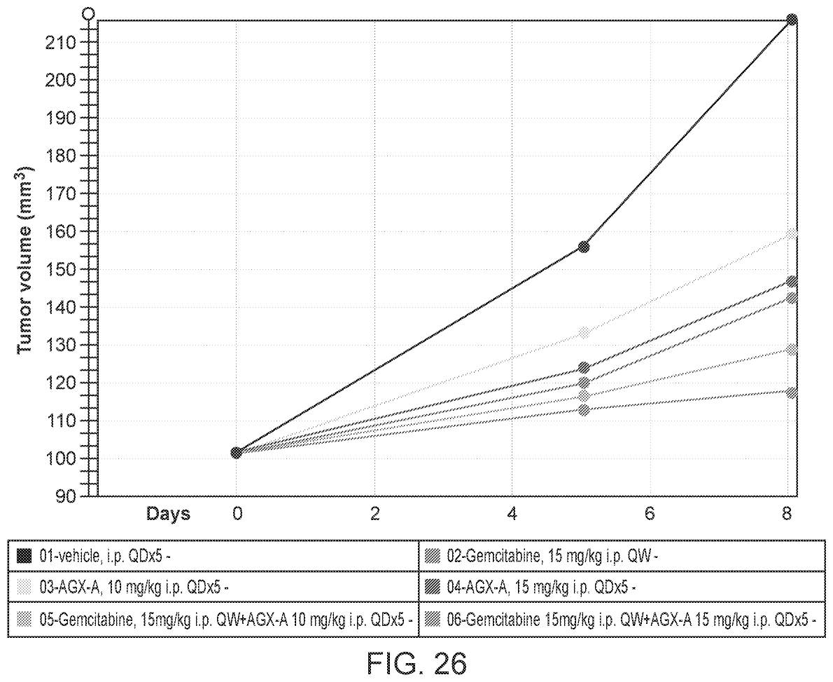

Tumor volume data from this Round 2 study is presented in FIG. 26. The data from this Round 2 study confirmed the significant potency and efficacy of anti-Id compounds of the present technology illustrated in the Round 1 study, both alone as well as part of a combination therapy.

### Example 22. Effects of AGX51 and Compounds of the Present Technology on Various Additional Cancer Cell Lines

The 4T1 murine mammary tumor cell line and nine breast cancer cell lines representing the major breast cancer subtypes (ER+, HER2+, and TNBC) including MDA-MB-157, MDA-MB-436, MDA-MB-231, MDA-MB-453, MDA-MB-361, BT-474, SK-BR-3, MCF-7 and T47-D will be grown in RPMI or DMEM (Dulbecco's Modified Eagle Medium) media supplemented with 10% FBS (fetal bovine serum), 1% penicillin-streptomycin and 1% L-Glutamine. Patient derived xenograft cell lines BR7, BR11, and IBT will be established directly from breast cancer bone metastases specimens surgically resected from patients with informed consent. BR7 and BR11 specimens will be obtained from ER positive (HER2 negative and PR negative) metastatic breast cancer patients, while the IBT specimen will be obtained from a metastatic, triple negative breast cancer patient. Fresh tumor tissues will be quickly washed with ice cold PBS and minced into about 1-3 mm pieces in MEM medium (without FBS) using sterile razor blades. A fraction of the minced original tumor tissues will be incubated with Collagenase/Hyaluronidase enzyme mix (1,000 Units, Voden Medical, Lombardia, Italy) in MEM medium without FBS (5 mL/250 mg tissue) for 2-4 hours. The dissociated tumor tissues will then be filtered throughout a 70 μm nylon filter, and cells will be concentrated by centrifugation at room temperature and seeded to derive respective primary cell cultures in MEM medium with 3% FBS (Sigma). The primary cell cultures will be transduced with the fluorescent td tomato-/EGFP-luciferase fusion protein expressing lentiviral vectors for 18-24 hours, the primary cells will then be maintained in MEM media supplemented with 3% FBS, 1% penicillin-streptomycin and 1% L-Glutamine. Aliquots of the primary cell cultures will also cryopreserved following a minimal number (3-4) of in vitro passages. The cells will be treated with either 100 μM DMSO, AGX51, or a compound of the present technology such as AGX-A. The cell morphology and growth will be monitored daily for 1 week by microscopy for changes in morphology or cell death. For IC₅₀ determination, cancer cell lines will be seeded in a 96 well plate (5000 cells per well). After overnight incubation, cells were treated with AGX51 (0, 5, 10, 20, 40, 60 PM) or with a compound of the present technology (0, 5, 10, 20, 40, 60 μM) and incubated for 24, 48 and 72 hours, each condition was done in triplicate. At each time point 40 μL of MTT reagent (5 mg/mL) will be added per well and the cells will be incubated for four hours. Following incubation, media will be aspirated and 200 μL DMSO added per well. Absorbance will then measured at 570 nm using a plate reader (Synergy 2, BioTek). For aid in future comparison with a compound of the present technology, the study was initially run as described above for AGX51 to provide the data shown in Table 3 below.

TABLE 3
IC50 values for AGX51 treated cell lines as determined by MTT assay.

| Cell Type | Cell line | IC50 value after 24 hours of treatment |
|---|---|---|
| cell line | 4T1 | 26.66 μM |
| TNBC cell line | MDA-MB-157 | 22.28 μM |
| | MDA-MB-436 | 30.91 μM |
| | MDA-MB-231 | NA (60 μM = 70%) |
| HER-2 +ve cell line | MDA-MB-453 | NA (60 μM = 57%) |
| | BT-474 | NA (60 μM = 53%) |
| | MDA-MB-361 | NA (60 μM = 53%) |
| | SK-BR-3 | 36.55 μM |
| ER +ve cell line | MCF-7 | 60 μM |
| | T47-D | NA (60 μM = 65%) |
| PDX cell line | PDX-BR7 | 10.89 μM |
| | PDX-IBT | 11.97 μM |
| | PDX-BR11 | 18.56 μM |

Legend: +, positive; TNBD, Triple negative breast cancer; ER, estrogen receptor; PDX, patient derived xenograft

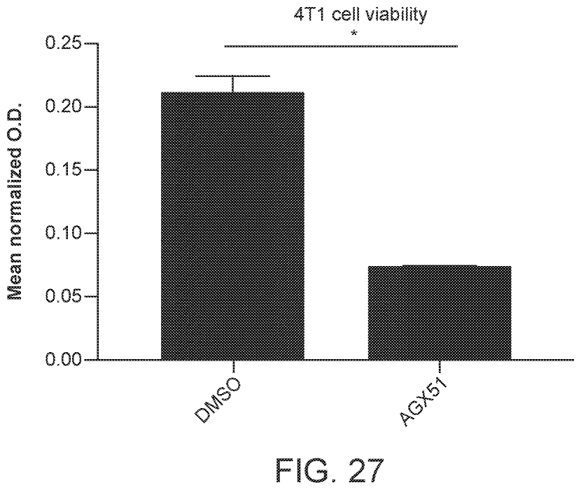

The effect of DMSO, AGX51, or a compound of the present technology (such as AGX-A) on 4T1 cells will also be assessed using Alamar blue viability assays according to manufacturer's instructions. Briefly, 5000 4T1 cells will be seeded in a 96-well plate, and the next day will be treated with 40 μM AGX51 for 24 hours, after which point a 1:10 dilution of Alamar blue cell viability reagent will be added to the cells and absorbance measured 2, 3, 4, 5 and 6 hours after addition of the reagent using a plate reader (Synergy 2, BioTek). For aid in future comparison with a compound of the present technology, such assays were run with AGX51 and the results provided in FIG. 27.

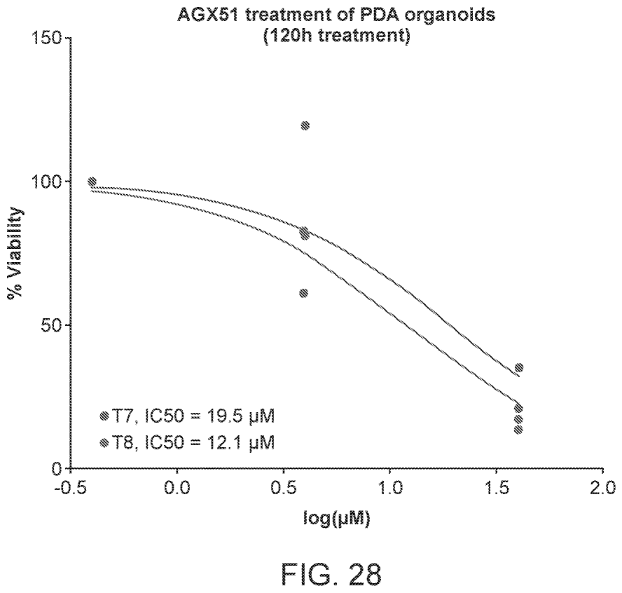

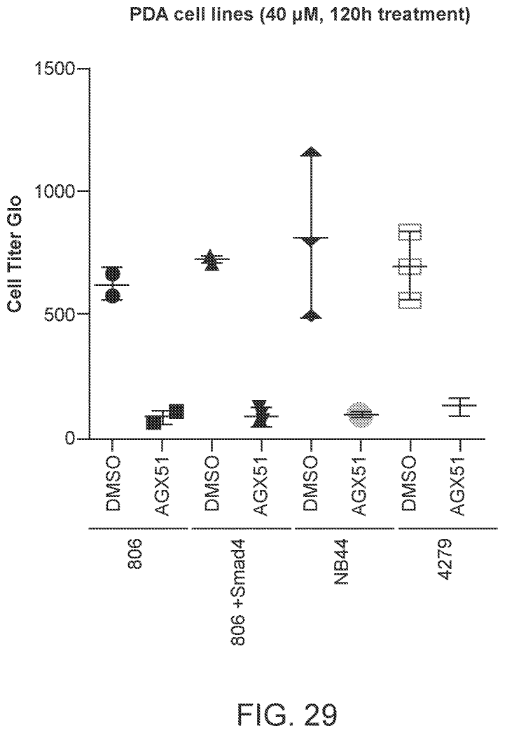

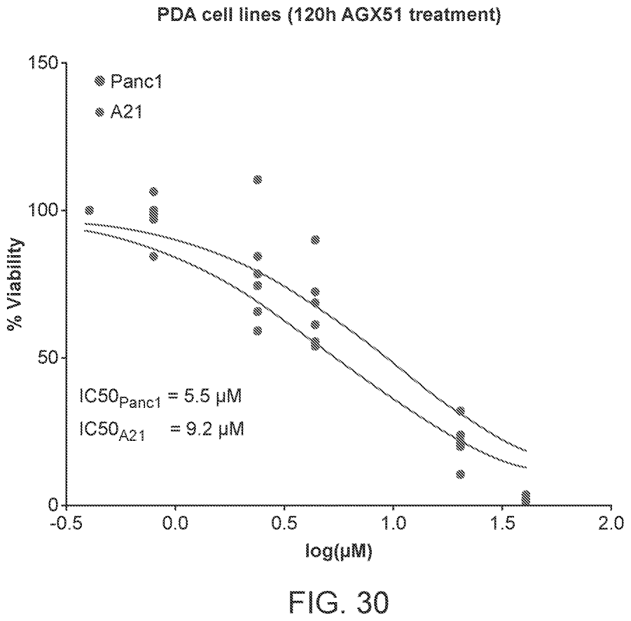

The effect of DMSO, AGX51, or a compound of the present technology (such as AGX-A) on pancreatic cancer cell and organoid lines will also examined. Lines to be examined are human pancreatic cancer cell line Panc1 and A21, mouse pancreatic cancer lines 806 (KrasG12D; Ink4a−/−; Smad4−/−), NB44 (KrasG12D; Ink4a−/−) and 4279 (KrasG12D; Ink4a−/−), and mouse pancreatic organoid cell lines T7 and T8. Pancreatic spheroids will be grown in Ultra Low Attachment Culture plates (Corning, Oneonta, N.Y., USA) in DMEM supplemented with Glutamax (2 mM) and heparin (5 μg/mL). Pancreatic organoids will be embedded in Matrigel with Advanced DMEM/F12 (12634-028, Gibco, Carlsbad, Calif.) supplemented with B-27 (12587-010, Life Technologies, Carlsbad, Calif.), HEPES (10 mM), 50% Wnt/R-spondin/Noggin-conditioned medium (ATCC, CRL-3276), Glutamax (2 mM, Invitrogen, Carlsbad, Calif.), N-acetyl-cysteine (1 mM, Sigma, St. Louis, Mo., USA), nicotinamide (10 mM, Sigma, St. Louis, Mo., USA), epidermal growth factor (50 ng/mL, Peprotech, Rocky Hill, N.J.), gastrin (10 nM, Sigma-Aldrich, St. Louis, Mo.), fibroblast growth factor-10 (100 ng/mL, Peprotech, Rocky Hill, N.J.), A83-01 (0.5 μM, Tocris, Bristol, United Kingdom) as described by Boj, S. F., et al., Cell 160: 324-338 (2015), incorporated herein by reference. All cell lines and organoids will be maintained at 37° C. and 5% CO₂. Cell viability will be measured using Cell Titer-Glo (Promega, Madison, Wis.) according to manufacturer's instructions. For aid in future comparison with a compound of the present technology, the study was initially performed with AGX51 and the results illustrated in FIG. 28 (mouse organoids), FIG. 29 (mouse cell lines 806, NB44, and 4279) and FIG. 30 (human cell lines Panc1 and A21). It is expected that compounds of the present technology will exhibit similar or significantly improved effects and providing yet further evidence that the compounds of the present technology are useful in treating cancers.

### Example 23. Effects of AGX51 and Compounds of the Present Technology on Cancer Cell Line Xenografts in Mice

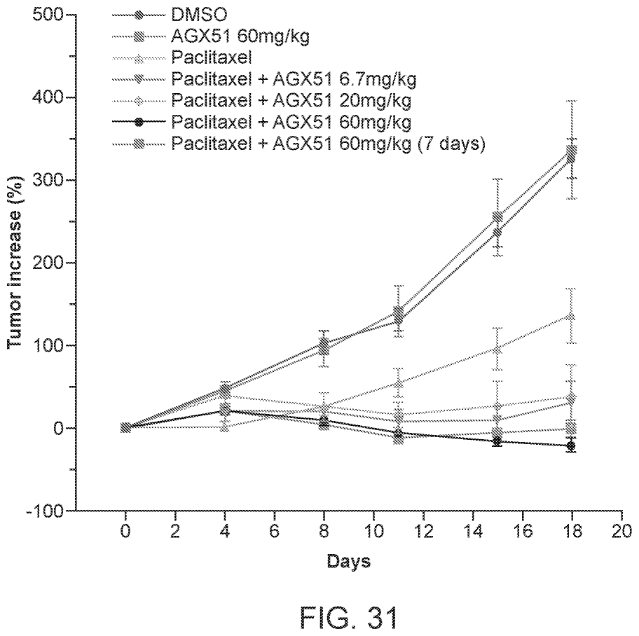

Effects of DMSO, AGX51, or a compound of the present technology (such as AGX-A) on primary tumors will be tested with and without paclitaxel in a primary tumor xenograft model using MDA-MB-231 cells. Orthotopic mammary fat pad tumors will be generated by injecting 5×10⁶ MDA-MB-231 cells (in 1:1 PBS:Matrigel) into the right caudal mammary fat pad of 8-12 week-old, female athymic nu/nu mice. Mice will be obtained from Simonsen Laboratories, Gilroy, Calif. Tumors will be allowed to grow to ˜100 mm³, at which point they will be divided into 12 groups of 5 mice with approximately the same tumor burden and treatment will then be initiated. Group 1 will be vehicle (DMSO, dosed q5d) control, Group 2 will receive 5 days of 15 mg/kg paclitaxel once a day, Group 3 will receive 19 days of 60 mg/kg AGX51 twice a day, Group 4 will receive 19 days of a combination of 15 mg/kg paclitaxel once a day and 6.7 mg/kg AGX51 twice a day, Group 5 will receive 19 days of a combination of 15 mg/kg paclitaxel once a day and 20 mg/kg AGX51 twice a day, Group 6 will receive 19 days of a combination of 15 mg/kg paclitaxel once a day and 60 mg/kg AGX51 twice a day, Group 7 will receive 15 mg/kg paclitaxel once a day for 19 days and concurrently receive 60 mg/kg AGX51 twice a day for the first 7 days, Group 8 will receive 19 days of 60 mg/kg of a compound of the present technology (e.g., AGX-A) twice a day, Group 9 will receive 19 days of a combination of 15 mg/kg paclitaxel once a day and 6.7 mg/kg AGX-A twice a day, Group 10 will receive 19 days of a combination of 15 mg/kg paclitaxel once a day and 20 mg/kg AGX-A twice a day, Group 11 will receive 19 days of a combination of 15 mg/kg paclitaxel once a day and 60 mg/kg AGX-A twice a day, Group 12 will receive 15 mg/kg paclitaxel once a day for 19 days and concurrently receive 60 mg/kg AGX-A twice a day for the first 7 days. Treatments will be administered i.p. Tumor volumes will be determined throughout the study using a digital caliper and the formula: tumor volume=½(length×width2) where the greatest longitudinal diameter is the length of the tumor and the greatest transverse diameter is the width. At study termination, the mice will be sacrificed by cervical dislocation. For aid in future comparison with a compound of the present technology, the study was initially performed with AGX51 (i.e., as provided for Groups 1-7, except that one mouse in Group 2 died during the study), where exemplary data of the effects of AGX51 with and without paclitaxel on primary tumors is shown in FIG. 31. It is expected that compounds of the present technology will exhibit similar or significantly improved effects and providing yet further evidence that the compounds of the present technology are useful in treating triple negative breast cancer.

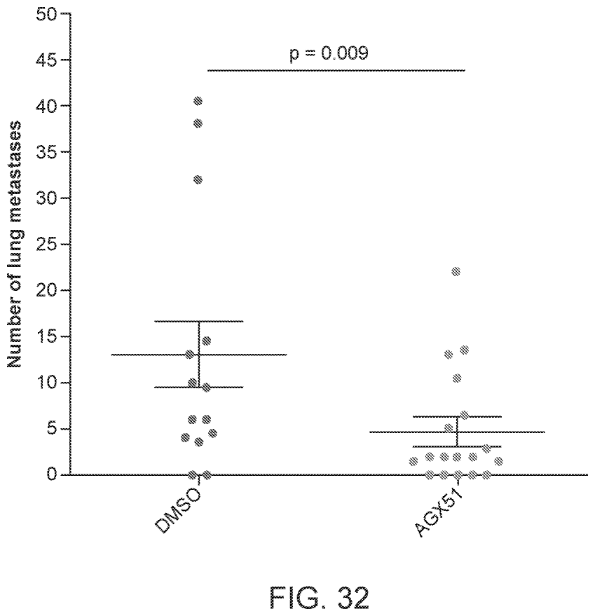

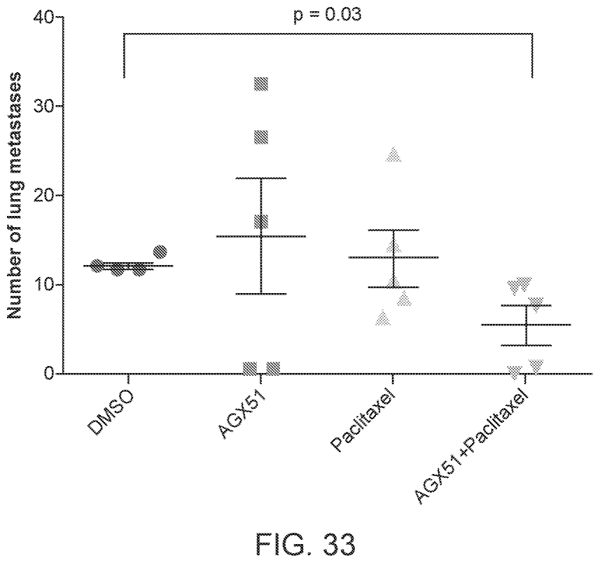

Effects of DMSO, AGX51, or a compound of the present technology (such as AGX-A) on metastasis will also be examined using a lung colonization model. Lung metastases will be generated by injecting 6-8 week-old, female, Balb/c mice with 50,000 luciferase-labeled 4T1 cells into the tail vein. Twenty-four hours after tail vein injections, mice will be treated once a day with DMSO, 50 mg/kg AGX51, or 50 mg/kg of e.g., AGX-A twice a day (at least five mice per treatment group) by i.p. injection. Development of lung metastases will be monitored using the IVIS-200 in vivo imaging system. For aid in future comparison with a compound of the present technology, the study was initially performed with DMSO and AGX51 where exemplary data of effects of AGX51 on lung colonization is presented in FIG. 32. Effects of DMSO AGX51, or a compound of the present technology (such as AGX-A) on established lung metastases will also be examined. Once evidence of lung metastases is observable by in vivo imaging, mice will be divided into groups of five mice each with approximately the same tumor burden per treatment group. The groups will be: Group 1 vehicle (DMSO) for 5 days, Group 2 50 mg/kg AGX51 twice a day for 5 days, Group 3 15 mg/kg paclitaxel once a day for 5 days, Group 4 a 5 day combination of 50 mg/kg AGX51 twice a day and 15 mg/kg paclitaxel once a day, Group 5 50 mg/kg of e.g., AGX-A twice a day for 5 days, Group 6 a 5 day combination of 50 mg/kg AGX-A twice a day and 15 mg/kg paclitaxel once a day. At the end of the experiments mice will be euthanized and tissues were collected for further analyses. Lung tumor burden will be quantified in a blinded fashion by a pathologist. For aid in future comparison with a compound of the present technology, the study was initially performed with DMSO and AGX51 where exemplary data of effects of AGX51 on established lung metastases is presented in FIG. 33. It is expected that compounds of the present technology will exhibit similar or significantly improved effects.

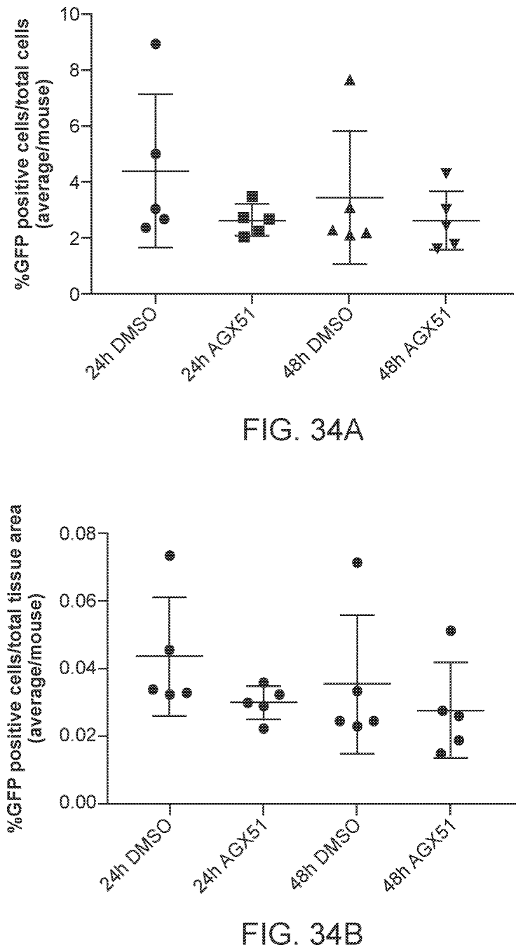

To assess the inhibition of the extravasation and initial seeding at the secondary site or the progression of extravasated cancer cell outgrowth into tumors, mice will be injected with GFP-labeled 4T1 cells from the tail vein, and treated with DMSO, AGX51 (50 mg/kg), or a compound of the present technology (e.g., AGX-A; 50 mg/kg) for 24 or 48 hours, then the lungs will be stained for GFP (i.e., tumor cells) and tumor cell number will be quantified. For aid in future comparison with a compound of the present technology, the study was initially performed with DMSO and AGX51 where exemplary data of effects of AGX51 on initial seeding of the cancer cells at the secondary site is shown in FIG. 34A in terms of total cells and in FIG. 34B in terms of total tissue area. It is expected that compounds of the present technology will exhibit similar or significantly improved effects.

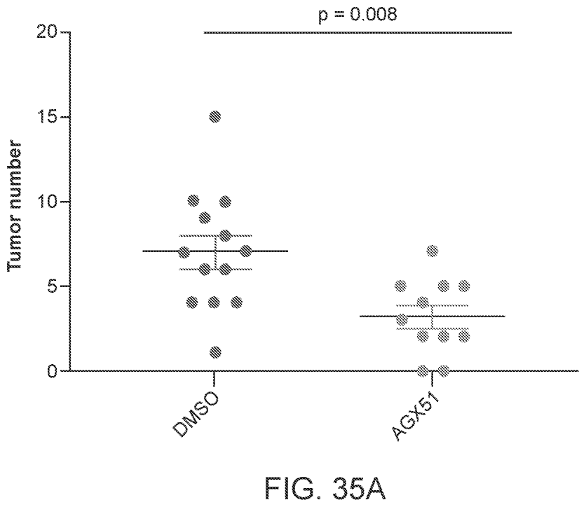

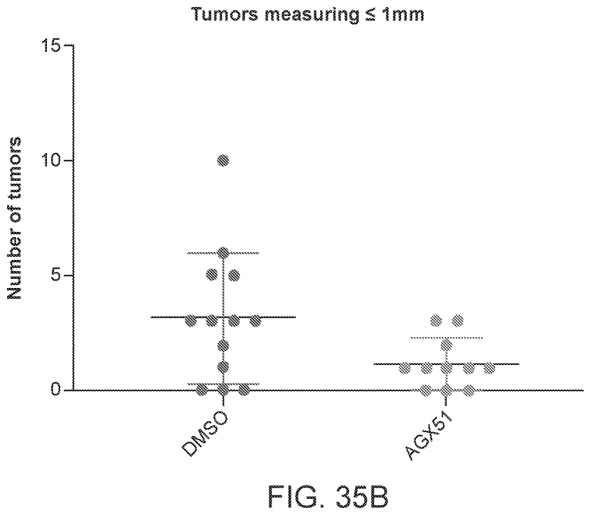

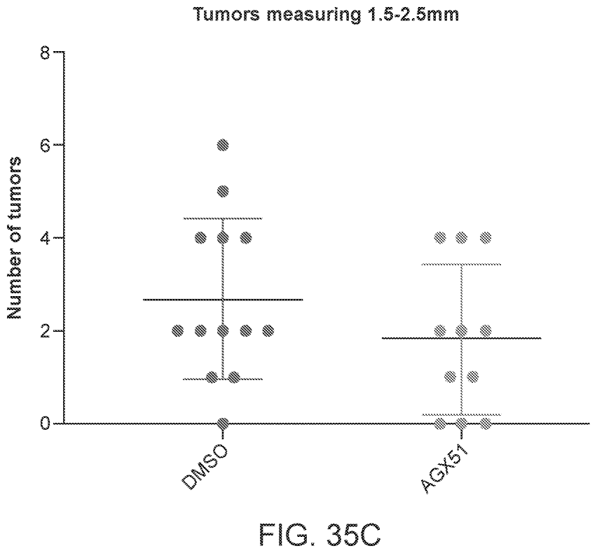

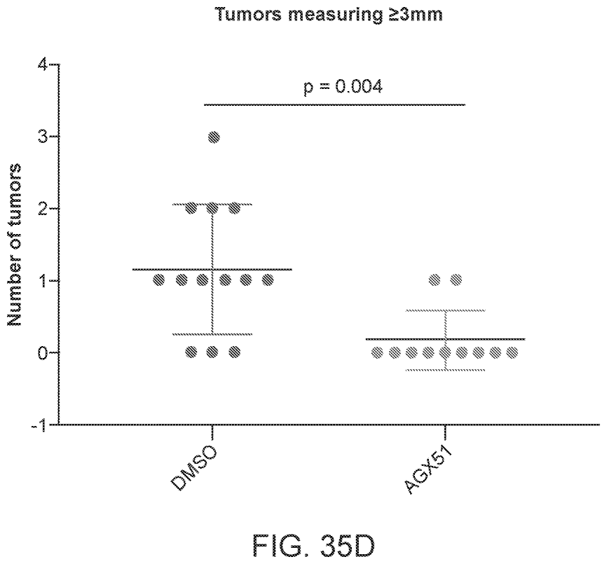

Effects of DMSO, AGX51, or a compound of the present technology (such as AGX-A) on sporadic tumor will be examined using the azoxymethane (AOM) colon tumor model, a chemically induced autochthonous model of adenoma. Spontaneous colon tumors will be induced by treating 30, 4-week old male A/J mice (Jackson Laboratory) with AOM (10 mg/kg; Sigma Aldrich) once a week by i.p. injection for 6 weeks. Mice will be maintained on AIN-93G purified diet (Research Diets) for the duration of the experiment. After a three-week treatment break mice will be treated i.p. for three weeks with DMSO, AGX51 (15 mg/kg) or a compound of the present technology (e.g., AGX-A; 15 mg/kg). Following the last injection, the mice will be euthanized and colon tumors will be formalin fixed to assess tumor burden. Tumor numbers and size will be determined in whole mounts of the tissues following methylene blue staining. For aid in future comparison with a compound of the present technology, the study was initially performed with DMSO and AGX51 where exemplary data of effects of AGX51 on sporadic tumor is shown FIGS. 35A-35D. In particular, AGX51 treatment resulted in a significant decrease in the number of colon tumors (p=0.008) as illustrated in FIG. 35A. In addition, the tumors from AGX51 group were smaller than those in the DMSO group (FIGS. 35B-35D), and this difference was significant when tumors measuring >3 mm were compared (p=0.004) as illustrated in FIG. 35D. It is expected that compounds of the present technology will exhibit similar or significantly improved effects.

### Example 24. Effects of AGX51 and Compounds of the Present Technology on Prostate Cancer Cell Lines

Prostate cancer cell lines DU145 and PC3 (in 10% BCS) will be cultured in Ham's F12 (Gibco, Carlsbad, Calif.) medium containing 10% BCS (Hyclone, Logan, Utah) and appropriate antibiotics (pen/strep, fungizaone, and gentamycin (Invitrogen Inc., Carlsbad, Calif.). All cells will be cultured at 37° C. in a fully humidified atmosphere containing 5% CO₂.

At 50% confluence, the cells will be treated with either 100 μM DMSO, AGX51, or a compound of the present technology (such as AGX-A). The cell morphology and growth will be monitored daily for 1 week by microscopy for changes in morphology or cell death. Apoptosis will be determined by measuring caspase 3 and caspase 7 activities using the Caspase-Glo 3/7 Assay system from Promega (Madison, Wis.).

It is expected that AGX51 and compounds of the present technology will show a pronounced effect on DU145 cells: after three days, the cells may appear very unhealthy and will be unable to proliferate as compared to the controls. Additionally, after six days, treatment of DU145 cells with either AGX51 a compound of the present technology will lead to cell death. It is expected that compounds of the present technology will exhibit similar or significantly improved effects as compared to AGX51.

The molecular mechanism underlying the effect of the compounds of the present technology on prostate cancer cells will be assessed by measuring the activity of the primary mediators of apoptosis, caspase 3/7. It is expected that low concentrations of a compound of the present technology will result in a significant increase in caspase 3/7 in both DU145 and PC3 cells, which will be higher than the caspase activity in cells treated with staurosporine (10 μm), a known apoptosis inducing agent.

### Example 25. Effects of AGX51 and Compounds of the Present Technology on Anti-Angiogenic Activity in a Matrigel Assay in Mice

VEGF-165 and FGF-2 treated Matrigel plugs will be implanted on Day 0 into C₅₇BL/6 mice. Mice will be treated with either vehicle, AGX51, or a compound of the present technology (such as AGX-A). The AGX51 and a compound of the present technology will be provided either in the plugs (25 μg/mg) or by daily ip treatment (30 or 100 mg/kg) for 10 days. Plugs will be harvested on Day 10, fixed and paraffin embedded. Three sections (5 M thickness) of each plug will be stained with an anti-CD31 antibody and counterstained with hematoxylin and eosin stain. CD31-positive microvessels will be counted for one entire cross-section per plug and the average micro-vessel density±SD vessels will be determined. Student's t-test will be used for statistical analysis.

It is anticipated that compared to vehicle control animals, treatments with AGX51 or a compound of the present technology will provide significant protection from new blood vessel formation. A typical picture of slices of the Matrigel plugs is expected to show the presence of complete blood vessels in vehicle alone control. In contrast, the presence of endothelial cells is expected to significantly decrease with the treatment with AGX51 or a compound of the present technology. It is expected that compounds of the present technology will exhibit similar or significantly improved effects as compared to AGX51.

### Example 26. Effects of AGX51 and Compounds of the Present Technology on Metastatic Activity in a LLC Mouse Model

Thirty C₅₇BL/6 mice will be implanted with 7.5×10⁵ LLC cancer cells/animal. Seven days after implantation, 5M/5F per group will be treated daily ip for 25 days with dosing vehicle (DMSO), AGX51, or a compound of the present technology (such as AGX-A). Fourteen days after implantation, another group of 5M/5F animals will be treated daily ip for 18 days with 50 mg/kg of AGX51 and another group of 5M/5F animals will be treated daily ip for 18 days with a compound of the present technology (such as AGX-A). Tumors will be measured three times from Day 7 to 14. On Day 14, the tumors will be excised. The animals will be necropsied for the presence of lung metastases on day 32, 18 days post excision. It is expected that the treatment with AGX51 and a compound of the present technology will profoundly diminish lung metastases. Further, it is expected that compounds of the present technology will exhibit similar or significantly improved effects as compared to AGX51.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A compound according to Formula I

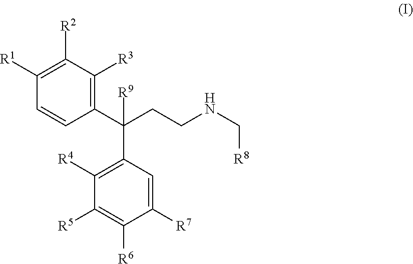

or a pharmaceutically acceptable salt and/or solvate thereof, wherein
   R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹⁰)(R¹¹)
   R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹²)(R¹³)
   R⁸ is aryl or heteroaryl;
   R⁹ is H, C₁-C₃ alkyl, or fluoro; and
   R¹⁰, R¹¹, R¹², and R¹³ are each independently C₁-C₃ alkyl.
B. The compound of Paragraph A, wherein R¹, R², and R³ are each independently H, C₁-C₃ alkyl, C₁-C₃ alkoxy, trifluoromethyl, trifluoromethoxy, halo, or —N(R¹⁰)(R¹¹)
C. The compound of Paragraph A or Paragraph B, wherein R¹, R², and R³ are each independently H, C₁-C₃ alkyl, C₁-C₃ alkoxy, halo, or —N(Me)₂.
D. The compound of any one of Paragraphs A-C, wherein R¹, R², and R³ are each independently H, methyl, methoxy, isopropyl, isopropoxy, fluoro, or —N(Me)₂.
E. The compound of any one of Paragraphs A-D, wherein R³ is methoxy.
F. The compound of any one of Paragraphs A-E, wherein R⁴, R⁵, R⁶, and R⁷ are each independently H, C₁-C₃ alkyl, C₁-C₃ alkoxy, trifluoromethyl, trifluoromethoxy, halo, or —N(R¹²)(R¹³).
G. The compound of any one of Paragraphs A-F, wherein R⁴, R⁵, R⁶, and R⁷ are each independently H, C₁-C₃ alkyl, C₁-C₃ alkoxy, halo, or —N(Me)₂.
H. The compound of any one of Paragraphs A-G, wherein R⁴, R⁵, R⁶, and R⁷ are each independently H, methyl, methoxy, isopropyl, isopropoxy, fluoro, or —N(Me)₂.
I. The compound of any one of Paragraphs A-H, wherein R⁶ is isopropoxy.
J. The compound of any one of Paragraphs A-I, where the compound is of Formula IA

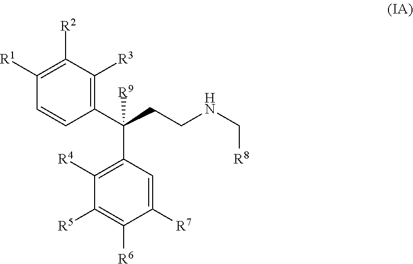

or a pharmaceutically acceptable salt and/or solvate thereof.
K. The compound of any one of Paragraphs A-I, wherein the compound is of Formula IB

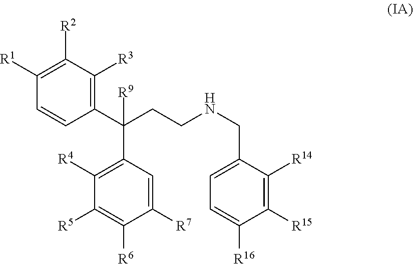

or a pharmaceutically acceptable salt and/or solvate thereof, wherein
   R¹⁴, R¹⁵, and R¹⁶ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.
L. The compound of Paragraph K, wherein R¹⁴, R¹⁵, and R¹⁶ are each independently H, C₁-C₃ alkyl, C₁-C₃ alkoxy, trifluoromethyl, trifluoromethoxy, halo, aryloxy, aryloyl, or —N(C₁-C₃ alkyl)₂.
M. The compound of Paragraph K or Paragraph L, wherein R¹⁴, R¹⁵, and R¹⁶ are each independently H, C₁-C₃ alkyl, C₁-C₃ alkoxy, trifluoromethyl, trifluoromethoxy, halo, or —N(Me)₂.
N. The compound of any one of Paragraphs K-M, wherein R¹⁴, R¹⁵, and R¹⁶ are each independently H, methyl, methoxy, isopropyl, isopropoxy, fluoro, or —N(Me)₂.
O. The compound of any one of Paragraphs K-N, wherein R¹, R¹⁵, and R¹⁶ are each independently H.
P. The compound of any one of Paragraphs A-O, wherein the compound is

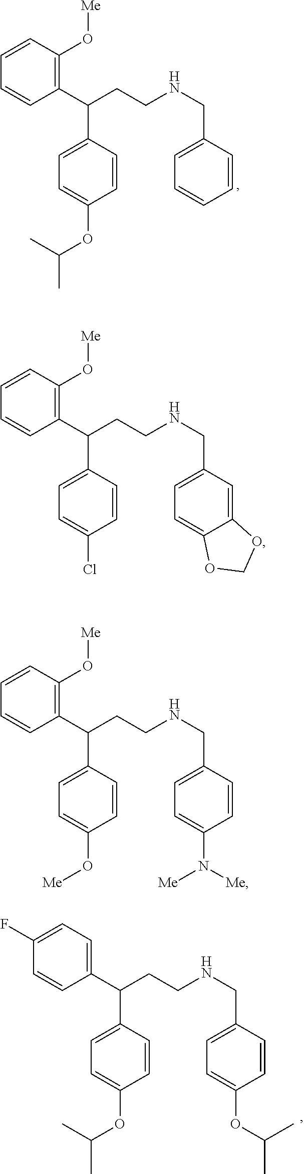

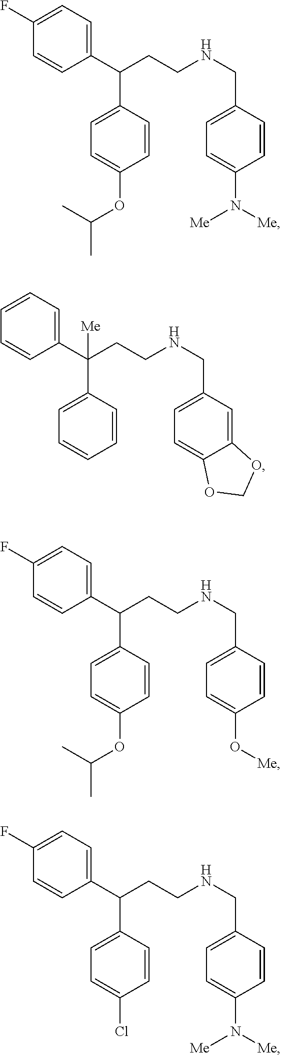

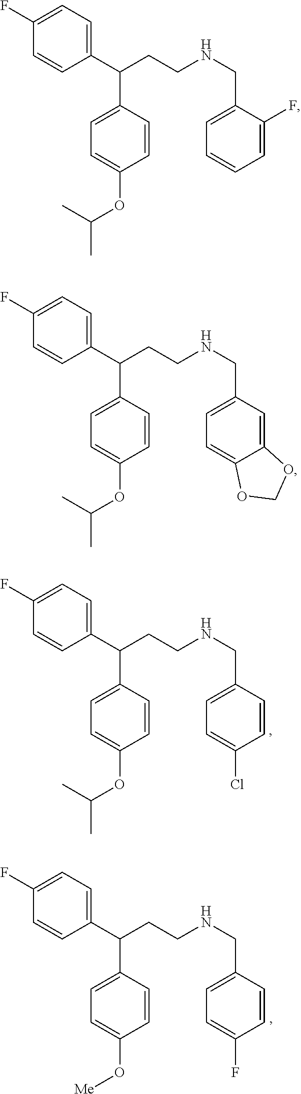 [SMILES: CC(C)Oc1ccc(C(CCNCc2ccccc2F)c2ccc(F)cc2)cc1 ; CC(C)Oc1ccc(C(CCNCc2ccc3c(c2)OCO3)c2ccc(F)cc2)cc1 ; CC(C)Oc1ccc(C(CCNCc2ccc(Cl)cc2)c2ccc(F)cc2)cc1 ; COc1ccc(C(CCNCc2ccc(F)cc2)c2ccc(F)cc2)cc1]

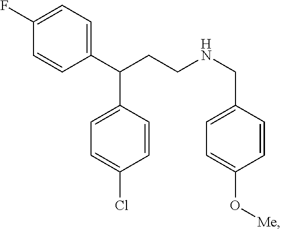 [SMILES: COc1ccc(CNCCC(c2ccc(F)cc2)c2ccc(Cl)cc2)cc1]

or a pharmaceutically acceptable salt and/or solvate thereof.
Q. The compound of any one of Paragraphs A-O, wherein the compound is of Formula IC

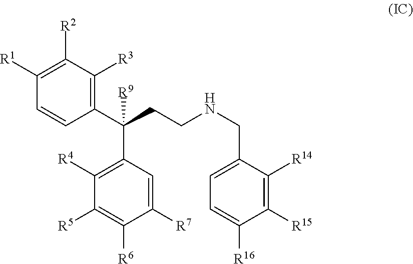

or a pharmaceutically acceptable salt and/or solvate thereof.
R. The compound of any one of Paragraphs A-Q, wherein the compound is

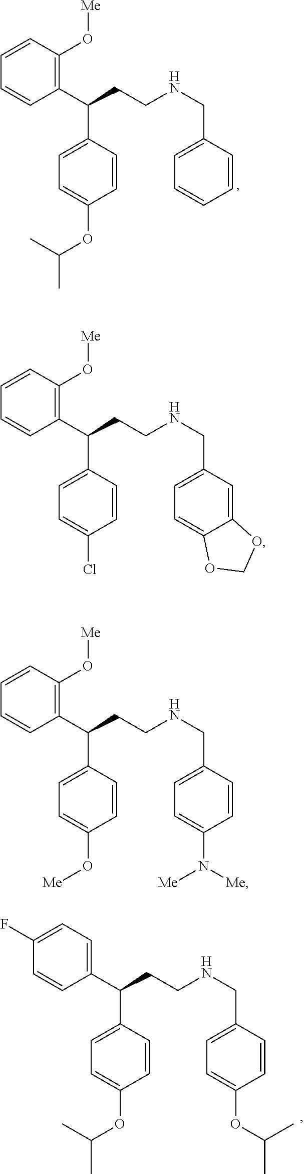

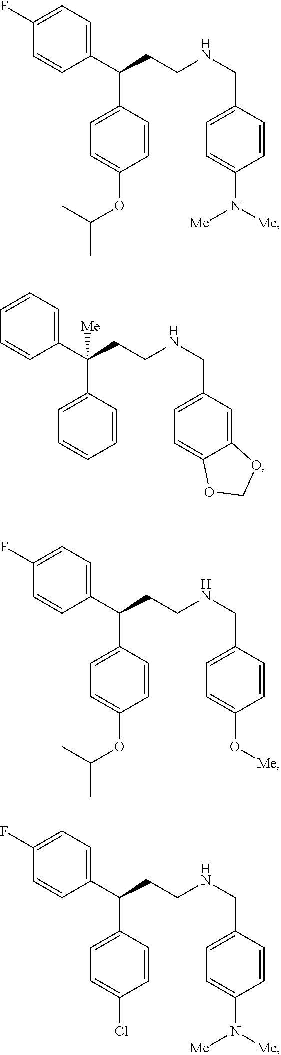

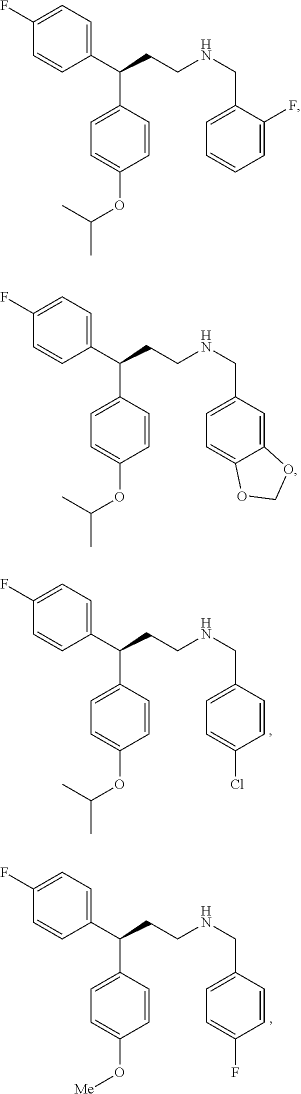 [SMILES: CC(C)Oc1ccc([C@H](CCNCc2ccccc2F)c2ccc(F)cc2)cc1 ; CC(C)Oc1ccc([C@H](CCNCc2ccc3c(c2)OCO3)c2ccc(F)cc2)cc1 ; CC(C)Oc1ccc([C@H](CCNCc2ccc(Cl)cc2)c2ccc(F)cc2)cc1 ; COc1ccc([C@H](CCNCc2ccc(F)cc2)c2ccc(F)cc2)cc1]

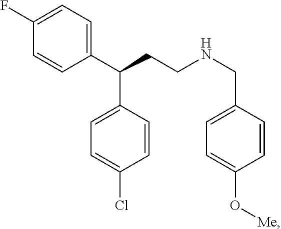 [SMILES: COc1ccc(CNCC[C@H](c2ccc(F)cc2)c2ccc(Cl)cc2)cc1]

or a pharmaceutically acceptable salt and/or solvate thereof.
S. A composition comprising
   a compound of any one of Paragraphs A-R; and
   a pharmaceutically acceptable carrier.
T. A pharmaceutical composition comprising
   an effective amount of a compound of any one of Paragraphs A-R for treating pathogenic cellular proliferation, angiogenesis, cancer, metastatic disease, and/or a pathogenic vascular proliferative disease in a subject; and
   a pharmaceutically acceptable carrier.
U. The pharmaceutical composition of Paragraph T, wherein the pathogenic vascular proliferative disease comprises pathogenic neovascularization associated with a cancer disease or condition.
V. The pharmaceutical composition of Paragraph T or Paragraph U, wherein the pathogenic vascular proliferative disease comprises an ocular disease.
W. The pharmaceutical composition of any one of Paragraphs T-V, wherein the pathogenic vascular proliferative disease is selected from the group consisting of age-related macular degeneration (AMD), diabetic retinopathy, retinopathy of prematurity, sickle cell retinopathy, retinal venous occlusive disease, central retinal vein occlusion (CRVO), branch retinal vein occlusion (BRVO), neovascular macular degeneration or an ocular cancer.
X. The pharmaceutical composition of any one of Paragraphs T-W, wherein the pathogenic vascular proliferative disease comprises age-related macular degeneration (AMD).
Y. The pharmaceutical composition of any one of Paragraphs T-X, wherein the pathogenic vascular proliferative disease comprises wet, exudative age-related macular degeneration.
Z. The pharmaceutical composition of any one of Paragraphs T-Y, wherein the cancer comprises cholangiocarcinoma, triple negative breast cancer, or colorectal cancer.
AA. The pharmaceutical composition of any one of Paragraphs T-Z, wherein the pharmaceutical composition is formulated for parenteral administration, intravenous administration, subcutaneous administration, and/or oral administration.
AB. The pharmaceutical composition of any one of Paragraphs T-AA, wherein the pharmaceutically acceptable carrier comprises 2-hydroxypropyl-β-cyclodextrin.
AC. The pharmaceutical composition of any one of Paragraphs T-AB, wherein the pharmaceutical composition is formulated for mammalian subject suffering a neoplasm.
AD. The pharmaceutical composition of any one of Paragraphs T-AC, wherein the pharmaceutical composition is formulated for a mammalian subject suffering or presenting with a history of neoplasm.
AE. The pharmaceutical composition of any one of Paragraphs T-AD, wherein the pharmaceutical composition is formulated for a mammalian subject suffering or previously treated into clinical remission for a neoplasm.
AF. The pharmaceutical composition of any one of Paragraphs T-AE, wherein the pharmaceutical composition is formulated for a mammalian subject suffering a condition mediated or contributed to by pathogenic neovascularization.
AG. A method for treating a condition in a subject, the method comprising administering a compound of any one of Paragraphs A-R to the subject in an amount effective to treat the condition, wherein the condition comprises one or more of pathogenic cellular proliferation, angiogenesis, cancer, metastatic disease, and/or a pathogenic vascular proliferative disease.
AH. The method of Paragraph AG, wherein the pathogenic vascular proliferative disease comprises pathogenic neovascularization associated with a cancer disease or condition.
AI. The method of Paragraph AG or Paragraph AH, wherein the pathogenic vascular proliferative disease comprises an ocular disease.
AJ. The method of any one of Paragraphs AG-AI, wherein the pathogenic vascular proliferative disease is selected from the group consisting of age-related macular degeneration (AMD), diabetic retinopathy, retinopathy of prematurity, sickle cell retinopathy, retinal venous occlusive disease, central retinal vein occlusion (CRVO), branch retinal vein occlusion (BRVO), neovascular macular degeneration or an ocular cancer.
AK. The method of any one of Paragraphs AG-AJ, wherein the pathogenic vascular proliferative disease comprises age-related macular degeneration (AMD).
AL. The method of any one of Paragraphs AG-AK, wherein the pathogenic vascular proliferative disease comprises wet, exudative age-related macular degeneration.
AM. The method of any one of Paragraphs AG-AL, wherein the cancer comprises cholangiocarcinoma, triple negative breast cancer, or colorectal cancer.
AN. The method of any one of Paragraphs AG-AM, wherein the pharmaceutical composition is formulated for parenteral administration, intravenous administration, subcutaneous administration, and/or oral administration.
   Other embodiments are set forth in the following claims, along with the full scope of equivalents to which such claims are entitled.

1. A compound according to Formula I 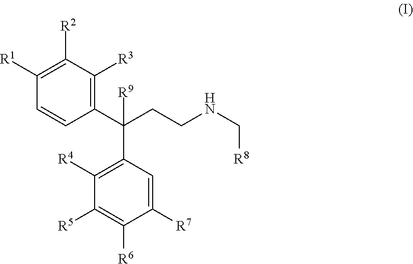
or a pharmaceutically acceptable salt and/or solvate thereof, wherein
R¹, R², and R³ are each independently H, C₁-C₃ alkyl, C₁-C₃ alkoxy, trifluoromethyl, trifluoromethoxy, trialkyl ammonium, pentafluorosulfanyl, halo, or —N(R¹⁰)(R¹¹);
R⁴, R⁵, R⁶, and R⁷ are each independently H, C₁-C₃ alkyl, C₁-C₃ alkoxy, trifluoromethyl, trifluoromethoxy, trialkyl ammonium, pentafluorosulfanyl, halo, or —N(R¹²)(R¹³);
R⁸ is aryl or heteroaryl;
R⁹ is H, C₁-C₃ alkyl, or fluoro; and
R¹⁰, R¹¹, R¹², and R¹³ are each independently C₁-C₃ alkyl.

2. The compound of claim 1, wherein R¹, R², and R³ are each independently H, C₁-C₃ alkyl, C₁-C₃ alkoxy, trifluoromethyl, trifluoromethoxy, halo, or —N(R¹⁰)(R¹¹).

3. (canceled)

4. The compound of claim 1, wherein R¹, R², and R³ are each independently H, methyl, methoxy, isopropyl, isopropoxy, fluoro, or —N(Me)₂.

5. The compound of claim 1, wherein R³ is methoxy.

6. The compound of claim 1, wherein R⁴, R⁵, R⁶, and R⁷ are each independently H, C₁-C₃ alkyl, C₁-C₃ alkoxy, trifluoromethyl, trifluoromethoxy, halo, or —N(R¹²)(R¹³).

7. (canceled)

8. The compound of claim 1, wherein R⁴, R⁵, R⁶, and R⁷ are each independently H, methyl, methoxy, isopropyl, isopropoxy, fluoro, or —N(Me)₂.

9. The compound of claim 1, wherein R⁶ is isopropoxy.

10. The compound of claim 1, where the compound is of Formula IA 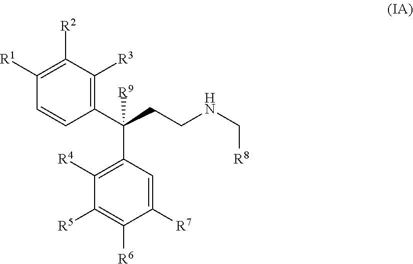
or a pharmaceutically acceptable salt and/or solvate thereof.

11. The compound of claim 1, wherein the compound is of Formula IB 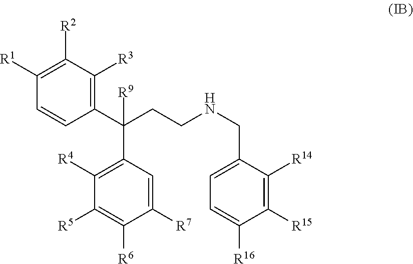
or a pharmaceutically acceptable salt and/or solvate thereof, wherein
R¹⁴, R¹⁵, and R¹⁶ are each independently H, C₁-C₃ alkyl, C₁-C₃ alkoxy, trifluoromethyl, trifluoromethoxy, trialkyl ammonium, pentafluorosulfanyl, halo, aryloxy, aryloyl, hydroxyl, amino, or amido.

12. The compound of claim 11, wherein R¹⁴, R¹⁵, and R¹⁶ are each independently H, C₁-C₃ alkyl, C₁-C₃ alkoxy, trifluoromethyl, trifluoromethoxy, halo, aryloxy, aryloyl, or —N(C₁-C₃ alkyl)₂.

13. The compound of claim 11, wherein R¹⁴, R¹⁵, and R¹⁶ are each independently H, C₁-C₃ alkyl, C₁-C₃ alkoxy, trifluoromethyl, trifluoromethoxy, halo, or —N(Me)₂.

14. The compound of claim 11, wherein R¹⁴, R¹⁵, and R¹⁶ are each independently H, methyl, methoxy, isopropyl, isopropoxy, fluoro, or —N(Me)₂.

15. The compound of claim 11, wherein R¹⁴, R¹⁵, and R¹⁶ are each independently H.

16. The compound of claim 1, wherein the compound is 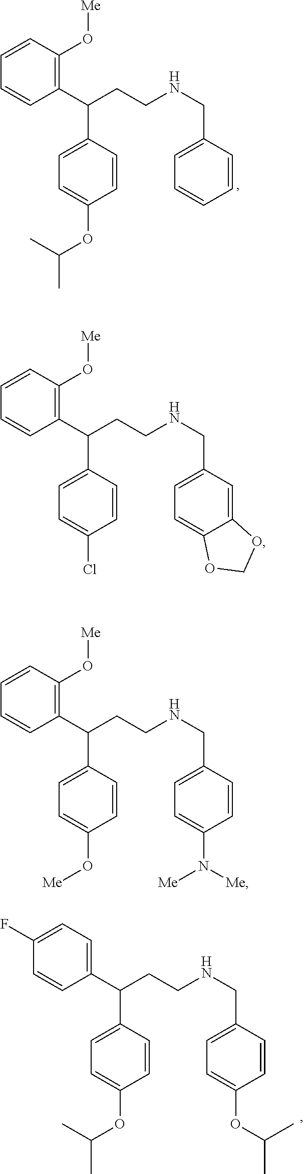 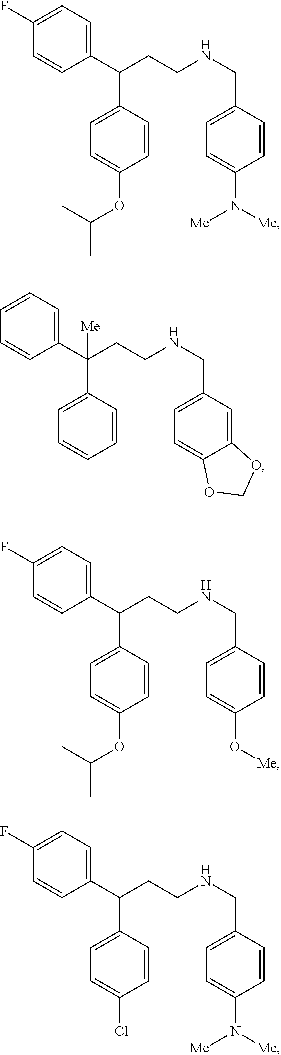 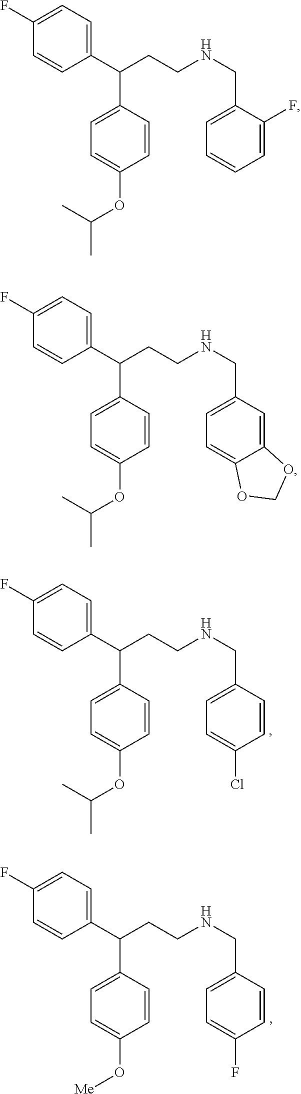 [SMILES: CC(C)Oc1ccc(C(CCNCc2ccccc2F)c2ccc(F)cc2)cc1 ; CC(C)Oc1ccc(C(CCNCc2ccc3c(c2)OCO3)c2ccc(F)cc2)cc1 ; CC(C)Oc1ccc(C(CCNCc2ccc(Cl)cc2)c2ccc(F)cc2)cc1 ; COc1ccc(C(CCNCc2ccc(F)cc2)c2ccc(F)cc2)cc1] 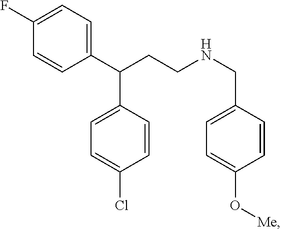 [SMILES: COc1ccc(CNCCC(c2ccc(F)cc2)c2ccc(Cl)cc2)cc1]
or a pharmaceutically acceptable salt and/or solvate thereof.

17. The compound of claim 11, wherein the compound is of Formula IC 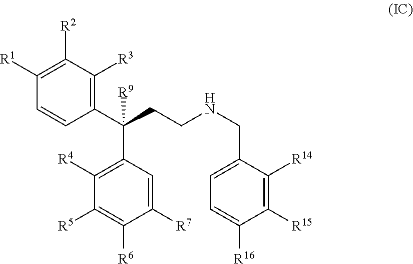
or a pharmaceutically acceptable salt and/or solvate thereof.

18. The compound of claim 1, wherein the compound is 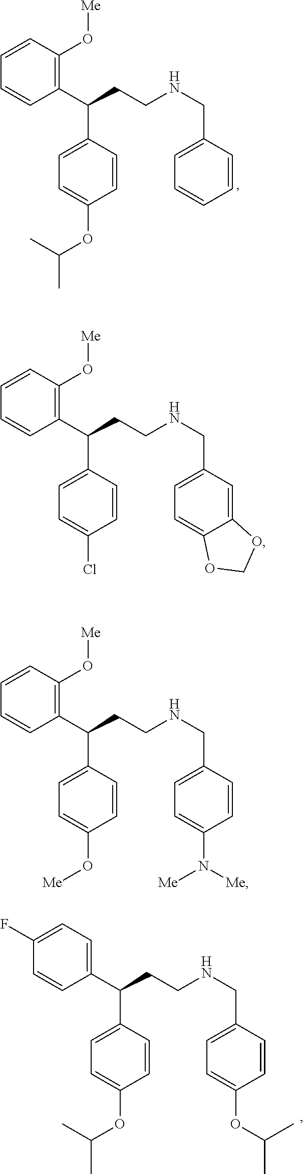 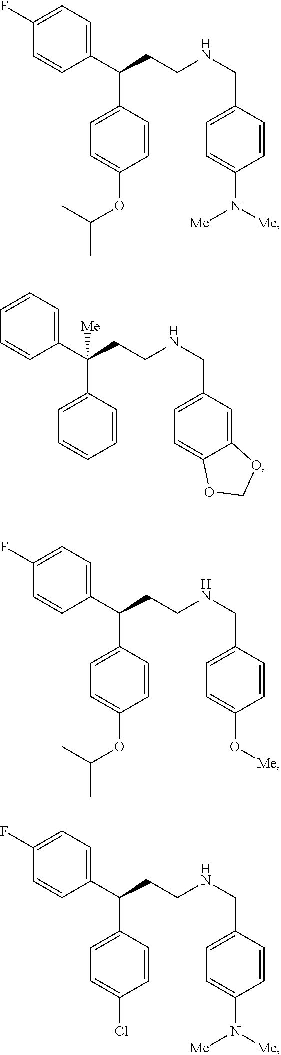 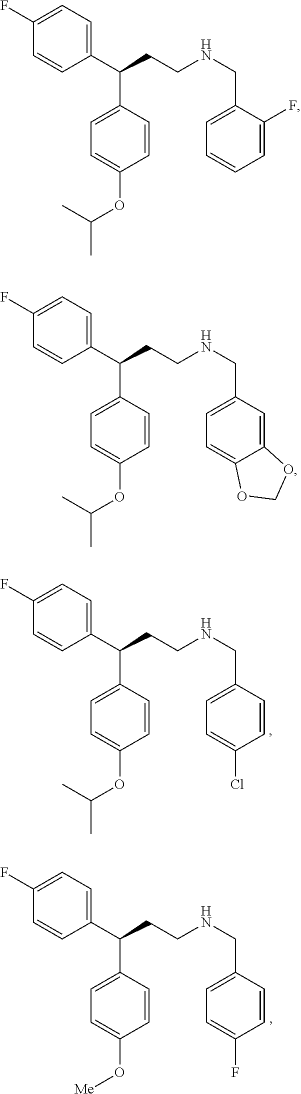 [SMILES: CC(C)Oc1ccc([C@H](CCNCc2ccccc2F)c2ccc(F)cc2)cc1 ; CC(C)Oc1ccc([C@H](CCNCc2ccc3c(c2)OCO3)c2ccc(F)cc2)cc1 ; CC(C)Oc1ccc([C@H](CCNCc2ccc(Cl)cc2)c2ccc(F)cc2)cc1 ; COc1ccc([C@H](CCNCc2ccc(F)cc2)c2ccc(F)cc2)cc1] 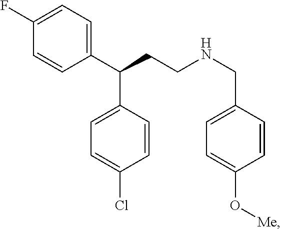 [SMILES: COc1ccc(CNCC[C@H](c2ccc(F)cc2)c2ccc(Cl)cc2)cc1]
or a pharmaceutically acceptable salt and/or solvate thereof.

19. A composition comprising
a compound of claim 1; and
a pharmaceutically acceptable carrier.

20. A pharmaceutical composition comprising
an effective amount of a compound of claim 1 for treating pathogenic cellular proliferation, angiogenesis, cancer, metastatic disease, and/or a pathogenic vascular proliferative disease in a subject; and
a pharmaceutically acceptable carrier.

21.-27. (canceled)

28. The pharmaceutical composition of claim 20, wherein the pharmaceutically acceptable carrier comprises 2-hydroxypropyl-β-cyclodextrin.

29.-32. (canceled)

33. A method for treating a condition in a subject, the method comprising administering a compound of claim 1 to the subject in an amount effective to treat the condition, wherein the condition comprises one or more of pathogenic cellular proliferation, angiogenesis, cancer, metastatic disease, and/or a pathogenic vascular proliferative disease.

34.-40. (canceled)
